SEQUENCE LISTING (383 sequences)

SEQ ID NO: 1 - protein, 31 aa, Homo sapiens
  MOD_RES (22)..(22): Phosphorylated Tyrosine
ALSEEMADTLEEGSASPTSPDYSLDSPGPEK

SEQ ID NO: 2 - protein, 16 aa, Homo sapiens
  MOD_RES (7)..(7): Phosphorylated Tyrosine
TATDEAYKDPSNLQGK

SEQ ID NO: 3 - protein, 20 aa, Homo sapiens
  MOD_RES (12)..(12): Phosphorylated Tyrosine
KHEALMSDLSAYGSSIQALR

SEQ ID NO: 4 - protein, 27 aa, Homo sapiens
  MOD_RES (19)..(19): Phosphorylated Tyrosine
QQVAPTDDETGKELVLALYDYQEKSPR

SEQ ID NO: 5 - protein, 10 aa, Homo sapiens
  MOD_RES (8)..(8): Phosphorylated Tyrosine
QGFVPAAYVK

SEQ ID NO: 6 - protein, 18 aa, Homo sapiens
  MOD_RES (9)..(9): Phosphorylated Tyrosine
LQTASDESYKDPTNIQSK

SEQ ID NO: 7 - protein, 12 aa, Homo sapiens
  MOD_RES (7)..(7): Phosphorylated Tyrosine
KMEEDIYTNLSK

SEQ ID NO: 8 - protein, 12 aa, Homo sapiens
  MOD_RES (8)..(8): Phosphorylated Tyrosine
ALPSMDKYAVFK

SEQ ID NO: 9 - protein, 19 aa, Homo sapiens
  MOD_RES (8)..(8): Phosphorylated Tyrosine
LYPELSQYMGLSLNEEEIR

SEQ ID NO: 10 - protein, 33 aa, Homo sapiens
  MOD_RES (16)..(16): Phosphorylated Tyrosine
AEGPTIKNDASQQTNYGVAVLDKEIIQLSDYLK

SEQ ID NO: 11 - protein, 19 aa, Homo sapiens
  MOD_RES (7)..(7): Phosphorylated Tyrosine
SLENQLYTCTQKYSPWGMK

SEQ ID NO: 12 - protein, 19 aa, Homo sapiens
  MOD_RES (13)..(13): Phosphorylated Tyrosine
SLENQLYTCTQKYSPWGMK

SEQ ID NO: 13 - protein, 11 aa, Homo sapiens
  MOD_RES (3)..(3): Phosphorylated Tyrosine
SQYEALKEDWR

SEQ ID NO: 14 - protein, 8 aa, Homo sapiens
  MOD_RES (3)..(3): Phosphorylated Tyrosine
ARYDFCAR

SEQ ID NO: 15 - protein, 25 aa, Homo sapiens
  MOD_RES (16)..(16): Phosphorylated Tyrosine
WLEQFEMALSNIRPDYADSNFHDFK

SEQ ID NO: 16 - protein, 8 aa, Homo sapiens
  MOD_RES (3)..(3): Phosphorylated Tyrosine
ARYDFCAR

SEQ ID NO: 17 - protein, 11 aa, Homo sapiens
  MOD_RES (7)..(7): Phosphorylated Tyrosine
RGDSQPYQALK

SEQ ID NO: 18 - protein, 34 aa, Homo sapiens
  MOD_RES (26)..(26): Phosphorylated Tyrosine
ADGATSDDLDLHDDRLSYLSAPGSEYSMYSTDSR

SEQ ID NO: 19 - protein, 21 aa, Homo sapiens
  MOD_RES (3)..(3): Phosphorylated Tyrosine
TLYRIPEPQKPQLKPPEDIVR

SEQ ID NO: 20 - protein, 26 aa, Homo sapiens
  MOD_RES (9)..(9): Phosphorylated Tyrosine
EKEPEEELYDLSKVVLAGGVAPALFR

SEQ ID NO: 21 - protein, 20 aa, Homo sapiens
  MOD_RES (15)..(15): Phosphorylated Tyrosine
PSPAISVSVSAPAFYAPQKK

SEQ ID NO: 22 - protein, 11 aa, Homo sapiens
  MOD_RES (9)..(9): Phosphorylated Tyrosine
NLYPSSSPYTR

SEQ ID NO: 23 - protein, 20 aa, Homo sapiens
  MOD_RES (18)..(18): Phosphorylated Tyrosine
ANLQQAIVTPLKPVDNTYYK

SEQ ID NO: 24 - protein, 19 aa, Homo sapiens
  MOD_RES (1)..(1): Phosphorylated Tyrosine
YKYRDLTVRETVNVITLYK

SEQ ID NO: 25 - protein, 19 aa, Homo sapiens
  MOD_RES (18)..(18): Phosphorylated Tyrosine
YKYRDLTVRETVNVITLYK

SEQ ID NO: 26 - protein, 12 aa, Homo sapiens
  MOD_RES (5)..(5): Phosphorylated Tyrosine
GILLYGPPGTGK

SEQ ID NO: 27 - protein, 13 aa, Homo sapiens
  MOD_RES (6)..(6): Phosphorylated Tyrosine
LDQLIYIPLPDEK

SEQ ID NO: 28 - protein, 13 aa, Homo sapiens
  MOD_RES (5)..(5): Phosphorylated Tyrosine
NCLVYSIPTNSSK

SEQ ID NO: 29 - protein, 16 aa, Homo sapiens
  MOD_RES (12)..(12): Phosphorylated Tyrosine
VILHLKEDQTEYLEER

SEQ ID NO: 30 - protein, 25 aa, Homo sapiens
  MOD_RES (17)..(17): Phosphorylated Tyrosine
ELDPTNMTYITNQAAVYFEKGDYNK

SEQ ID NO: 31 - protein, 25 aa, Homo sapiens
  MOD_RES (23)..(23): Phosphorylated Tyrosine
ELDPTNMTYITNQAAVYFEKGDYNK

SEQ ID NO: 32 - protein, 12 aa, Homo sapiens
  MOD_RES (10)..(10): Phosphorylated Tyrosine
AAALEAMKDYTK

SEQ ID NO: 33 - protein, 13 aa, Homo sapiens
  MOD_RES (9)..(9): Phosphorylated Tyrosine
MRAEDGENYDIKK

SEQ ID NO: 34 - protein, 18 aa, Homo sapiens
  MOD_RES (14)..(14): Phosphorylated Tyrosine
ILENEKDLEEAEEYKEAR

SEQ ID NO: 35 - protein, 12 aa, Homo sapiens
  MOD_RES (10)..(10): Phosphorylated Tyrosine
KDYNEAYNYYTK

SEQ ID NO: 36 - protein, 21 aa, Homo sapiens
  MOD_RES (19)..(19): Phosphorylated Tyrosine
KLDDAIEDCTNAVKLDDTYIK

SEQ ID NO: 37 - protein, 17 aa, Homo sapiens
  MOD_RES (13)..(13): Phosphorylated Tyrosine
RPWVEYENVRQEYEEVK

SEQ ID NO: 38 - protein, 8 aa, Homo sapiens
  MOD_RES (6)..(6): Phosphorylated Tyrosine
SMMSAYER

SEQ ID NO: 39 - protein, 11 aa, Homo sapiens
  MOD_RES (6)..(6): Phosphorylated Tyrosine
SMMSSYSAADR

SEQ ID NO: 40 - protein, 14 aa, Homo sapiens
  MOD_RES (6)..(6): Phosphorylated Tyrosine
EADSVYGEWVPVEK

SEQ ID NO: 41 - protein, 20 aa, Homo sapiens
  MOD_RES (9)..(9): Phosphorylated Tyrosine
IKHFDGEDYTCITFQPDLSK

SEQ ID NO: 42 - protein, 12 aa, Homo sapiens
  MOD_RES (4)..(4): Phosphorylated Tyrosine
HVDYVVDQVVGK

SEQ ID NO: 43 - protein, 12 aa, Homo sapiens
  MOD_RES (7)..(7): Phosphorylated Tyrosine
KVEEVVYDLSIR

SEQ ID NO: 44 - protein, 20 aa, Homo sapiens
  MOD_RES (15)..(15): Phosphorylated Tyrosine
QVYDGFSFIGNTGPYEVSKK

SEQ ID NO: 45 - protein, 14 aa, Homo sapiens
  MOD_RES (8)..(8): Phosphorylated Tyrosine
GGPLNKEYTNWEAK

SEQ ID NO: 46 - protein, 16 aa, Homo sapiens
  MOD_RES (6)..(6): Phosphorylated Tyrosine
DMESDYSGQGVDQLQR

SEQ ID NO: 47 - protein, 18 aa, Homo sapiens
  MOD_RES (15)..(15): Phosphorylated Tyrosine
KFNHDGEEEEEDDDYGSR

SEQ ID NO: 48 - protein, 15 aa, Homo sapiens
  MOD_RES (8)..(8): Phosphorylated Tyrosine
RRDEDMLYSPELAQR

SEQ ID NO: 49 - protein, 9 aa, Homo sapiens
  MOD_RES (4)..(4): Phosphorylated Tyrosine
DDVYEAFMK

SEQ ID NO: 50 - protein, 11 aa, Homo sapiens
  MOD_RES (6)..(6): Phosphorylated Tyrosine
DMESYYQEIGR

SEQ ID NO: 51 - protein, 14 aa, Homo sapiens
  MOD_RES (10)..(10): Phosphorylated Tyrosine
RVVSEDTSHYILIK

SEQ ID NO: 52 - protein, 10 aa, Homo sapiens
  MOD_RES (4)..(4): Phosphorylated Tyrosine
KFTYLGSQDR

SEQ ID NO: 53 - protein, 31 aa, Homo sapiens
  MOD_RES (19)..(19): Phosphorylated Tyrosine
FLENGSQEDLLHGNPGSTYLASNSTSAPNWK

SEQ ID NO: 54 - protein, 14 aa, Homo sapiens
  MOD_RES (4)..(4): Phosphorylated Tyrosine
DAIYSHKNCPYDAK

SEQ ID NO: 55 - protein, 15 aa, Homo sapiens
  MOD_RES (11)..(11): Phosphorylated Tyrosine
QVLVKAELEKYQQYK

SEQ ID NO: 56 - protein, 11 aa, Homo sapiens
  MOD_RES (9)..(9): Phosphorylated Tyrosine
DATNIQRKYLK

SEQ ID NO: 57 - protein, 22 aa, Homo sapiens
  MOD_RES (3)..(3): Phosphorylated Tyrosine
KGYVSLEDYTAFLIDKESENIK

SEQ ID NO: 58 - protein, 9 aa, Homo sapiens
  MOD_RES (5)..(5): Phosphorylated Tyrosine
ITDLYTDLR

SEQ ID NO: 59 - protein, 13 aa, Homo sapiens
  MOD_RES (9)..(9): Phosphorylated Tyrosine
MLTAQDMSYDEAR

SEQ ID NO: 60 - protein, 15 aa, Homo sapiens
  MOD_RES (5)..(5): Phosphorylated Tyrosine
VDKLYAGLKDLAEER

SEQ ID NO: 61 - protein, 26 aa, Homo sapiens
  MOD_RES (12)..(12): Phosphorylated Tyrosine
TSSISGPLSPAYTGQVPYNYNQLEGR

SEQ ID NO: 62 - protein, 26 aa, Homo sapiens
  MOD_RES (18)..(18): Phosphorylated Tyrosine
TSSISGPLSPAYTGQVPYNYNQLEGR

SEQ ID NO: 63 - protein, 20 aa, Homo sapiens
  MOD_RES (13)..(13): Phosphorylated Tyrosine
EVVAASHELGQDYEHVTMLR

SEQ ID NO: 64 - protein, 13 aa, Homo sapiens
  MOD_RES (9)..(9): Phosphorylated Tyrosine
NSLQNLSQYPMMR

SEQ ID NO: 65 - protein, 14 aa, Homo sapiens
  MOD_RES (10)..(10): Phosphorylated Tyrosine
GASQAGMTGYGMPR

SEQ ID NO: 66 - protein, 17 aa, Homo sapiens
  MOD_RES (14)..(14): Phosphorylated Tyrosine
KGIWLEAGKALDYYMLR

SEQ ID NO: 67 - protein, 15 aa, Homo sapiens
  MOD_RES (3)..(3): Phosphorylated Tyrosine
SGYSSPGSPGTPGSR

SEQ ID NO: 68 - protein, 12 aa, Homo sapiens
  MOD_RES (5)..(5): Phosphorylated Tyrosine
VQIVYKPVDLSK

SEQ ID NO: 69 - protein, 13 aa, Homo sapiens
  MOD_RES (8)..(8): Phosphorylated Tyrosine
LGVIVSAYMHYSK

SEQ ID NO: 70 - protein, 13 aa, Homo sapiens
  MOD_RES (11)..(11): Phosphorylated Tyrosine
LGVIVSAYMHYSK

SEQ ID NO: 71 - protein, 10 aa, Homo sapiens
  MOD_RES (7)..(7): Phosphorylated Tyrosine
RTQYSCYCCK

SEQ ID NO: 72 - protein, 12 aa, Homo sapiens
  MOD_RES (5)..(5): Phosphorylated Tyrosine
DREDYVPYTGEK

SEQ ID NO: 73 - protein, 15 aa, Homo sapiens
  MOD_RES (10)..(10): Phosphorylated Tyrosine
HIAEEADRKYEEVAR

SEQ ID NO: 74 - protein, 12 aa, Homo sapiens
  MOD_RES (5)..(5): Phosphorylated Tyrosine
INVYYNEATGGK

SEQ ID NO: 75 - protein, 12 aa, Homo sapiens
  MOD_RES (10)..(10): Phosphorylated Tyrosine
KLLIAQEPLYQC

SEQ ID NO: 76 - protein, 23 aa, Homo sapiens
  MOD_RES (3)..(3): Phosphorylated Tyrosine
LHYPMVEYCIPTTSGEDVRDFTK

SEQ ID NO: 77 - protein, 19 aa, Homo sapiens
  MOD_RES (8)..(8): Phosphorylated Tyrosine
LHYPMVEYCIPTTSGEDVR

SEQ ID NO: 78 - protein, 12 aa, Homo sapiens
  MOD_RES (6)..(6): Phosphorylated Tyrosine
ATVMLYDDGNKR

SEQ ID NO: 79 - protein, 25 aa, Homo sapiens
  MOD_RES (20)..(20): Phosphorylated Tyrosine
SSSSVTTSETQPCTPSSSDYSDLQR

SEQ ID NO: 80 - protein, 10 aa, Homo sapiens
  MOD_RES (9)..(9): Phosphorylated Tyrosine
NLQEAEEWYK

SEQ ID NO: 81 - protein, 13 aa, Homo sapiens
  MOD_RES (7)..(7): Phosphorylated Tyrosine
FYTNPSYFFDLWK

SEQ ID NO: 82 - protein, 9 aa, Homo sapiens
  MOD_RES (3)..(3): Phosphorylated Tyrosine
YEYQPFAGK

SEQ ID NO: 83 - protein, 16 aa, Homo sapiens
  MOD_RES (2)..(2): Phosphorylated Tyrosine
MYVPMTEDIYNAISAK

SEQ ID NO: 84 - protein, 16 aa, Homo sapiens
  MOD_RES (10)..(10): Phosphorylated Tyrosine
MYVPMTEDIYNAISAK

SEQ ID NO: 85 - protein, 18 aa, Homo sapiens
  MOD_RES (16)..(16): Phosphorylated Tyrosine
LGQSDPAPLQHQMDIYQK

SEQ ID NO: 86 - protein, 12 aa, Homo sapiens
  MOD_RES (4)..(4): Phosphorylated Tyrosine
HVDYVADQIVTK

SEQ ID NO: 87 - protein, 12 aa, Homo sapiens
  MOD_RES (1)..(1): Phosphorylated Tyrosine
YNPYTRSIQILK

SEQ ID NO: 88 - protein, 12 aa, Homo sapiens
  MOD_RES (4)..(4): Phosphorylated Tyrosine
YNPYTRSIQILK

SEQ ID NO: 89 - protein, 20 aa, Homo sapiens
  MOD_RES (15)..(15): Phosphorylated Tyrosine
VPADTEVVCAPPTAYIDFAR

SEQ ID NO: 90 - protein, 15 aa, Homo sapiens
  MOD_RES (5)..(5): Phosphorylated Tyrosine
VVLAYEPVWAIGTGK

SEQ ID NO: 91 - protein, 10 aa, Homo sapiens
  MOD_RES (3)..(3): Phosphorylated Tyrosine
GMYDVGLPSR

SEQ ID NO: 92 - protein, 32 aa, Homo sapiens
  MOD_RES (17)..(17): Phosphorylated Tyrosine
TNPTEPVGVVCRVDGVYQVVEYSEISLATAQK

SEQ ID NO: 93 - protein, 12 aa, Homo sapiens
  MOD_RES (7)..(7): Phosphorylated Tyrosine
GGTLTQYEGKLR

SEQ ID NO: 94 - protein, 17 aa, Homo sapiens
  MOD_RES (11)..(11): Phosphorylated Tyrosine
AALGPLVTGLYDVQAFK

SEQ ID NO: 95 - protein, 27 aa, Homo sapiens
  MOD_RES (10)..(10): Phosphorylated Tyrosine
YIANLGHGLYPDMDPEHVGAFVDAVHK

SEQ ID NO: 96 - protein, 16 aa, Homo sapiens
  MOD_RES (9)..(9): Phosphorylated Tyrosine
AAWGEETDYTPVWCMR

SEQ ID NO: 97 - protein, 38 aa, Homo sapiens
  MOD_RES (13)..(13): Phosphorylated Tyrosine
SLGNVIDPLDVIYGISLQGLHNQLLNSNLDPSEVEKAK

SEQ ID NO: 98 - protein, 21 aa, Homo sapiens
  MOD_RES (13)..(13): Phosphorylated Tyrosine
SHILYDRAGEEHYNCISALHK

SEQ ID NO: 99 - protein, 16 aa, Homo sapiens
  MOD_RES (15)..(15): Phosphorylated Tyrosine
EFIESLQLKPGQVVYK

SEQ ID NO: 100 - protein, 30 aa, Homo sapiens
  MOD_RES (26)..(26): Phosphorylated Tyrosine
AVFGHPFSLGWASPFATPDQGKADPYQYVV

SEQ ID NO: 101 - protein, 10 aa, Homo sapiens
  MOD_RES (6)..(6): Phosphorylated Tyrosine
VLNELYSVMK

SEQ ID NO: 102 - protein, 15 aa, Homo sapiens
  MOD_RES (12)..(12): Phosphorylated Tyrosine
YPLIIVSEEIEYLLK

SEQ ID NO: 103 - protein, 21 aa, Homo sapiens
  MOD_RES (4)..(4): Phosphorylated Tyrosine
GNLYSFGCPEYGQLGHNSDGK

SEQ ID NO: 104 - protein, 11 aa, Homo sapiens
  MOD_RES (9)..(9): Phosphorylated Tyrosine
VFSWGFGGYGR

SEQ ID NO: 105 - protein, 17 aa, Homo sapiens
  MOD_RES (13)..(13): Phosphorylated Tyrosine
VGWFPANYVEEDYSEYC

SEQ ID NO: 106 - protein, 7 aa, Homo sapiens
  MOD_RES (2)..(2): Phosphorylated Tyrosine
LYPVSRF

SEQ ID NO: 107 - protein, 16 aa, Homo sapiens
  MOD_RES (14)..(14): Phosphorylated Tyrosine
MVVLECMLKKDLIYNK

SEQ ID NO: 108 - protein, 12 aa, Homo sapiens
  MOD_RES (7)..(7): Phosphorylated Tyrosine
DNCQYVYNVDQR

SEQ ID NO: 109 - protein, 12 aa, Homo sapiens
  MOD_RES (8)..(8): Phosphorylated Tyrosine
DQDELNPYAAWR

SEQ ID NO: 110 - protein, 14 aa, Homo sapiens
  MOD_RES (1)..(1): Phosphorylated Tyrosine
YFVDKAAARKVLSR

SEQ ID NO: 111 - protein, 33 aa, Homo sapiens
  MOD_RES (31)..(31): Phosphorylated Tyrosine
AQDEIPALSVSRPQTGLSFLGPEPEDLEDLYSR

SEQ ID NO: 112 - protein, 12 aa, Homo sapiens
  MOD_RES (5)..(5): Phosphorylated Tyrosine
GVLMYGPPGCGK

SEQ ID NO: 113 - protein, 10 aa, Homo sapiens
  MOD_RES (9)..(9): Phosphorylated Tyrosine
KDEQEHEFYK

SEQ ID NO: 114 - protein, 22 aa, Homo sapiens
  MOD_RES (7)..(7): Phosphorylated Tyrosine
VNESSHYDLAFTDVHFKPGQIR

SEQ ID NO: 115 - protein, 24 aa, Homo sapiens
  MOD_RES (17)..(17): Phosphorylated Tyrosine
LDSSDIYNELKETYPNYLPLYVAR

SEQ ID NO: 116 - protein, 14 aa, Homo sapiens
  MOD_RES (10)..(10): Phosphorylated Tyrosine
GEVLEGSNAYYCEK

SEQ ID NO: 117 - protein, 12 aa, Homo sapiens
  MOD_RES (2)..(2): Phosphorylated Tyrosine
VYDQTNPYTDVR

SEQ ID NO: 118 - protein, 12 aa, Homo sapiens
  MOD_RES (5)..(5): Phosphorylated Tyrosine
CCMPYTPICIAK

SEQ ID NO: 119 - protein, 17 aa, Homo sapiens
  MOD_RES (10)..(10): Phosphorylated Tyrosine
VFPGSTTEDYNLIVIER

SEQ ID NO: 120 - protein, 18 aa, Homo sapiens
  MOD_RES (3)..(3): Phosphorylated Tyrosine
TLYEHYSGGESHNSSSSK

SEQ ID NO: 121 - protein, 18 aa, Homo sapiens
  MOD_RES (6)..(6): Phosphorylated Tyrosine
TLYEHYSGGESHNSSSSK

SEQ ID NO: 122 - protein, 9 aa, Homo sapiens
  MOD_RES (5)..(5): Phosphorylated Tyrosine
GRGEYFAIK

SEQ ID NO: 123 - protein, 14 aa, Homo sapiens
  MOD_RES (11)..(11): Phosphorylated Tyrosine
EIGHGSFGAVYFAR

SEQ ID NO: 124 - protein, 12 aa, Homo sapiens
  MOD_RES (4)..(4): Phosphorylated Tyrosine
KMSYSGKQSNEK

SEQ ID NO: 125 - protein, 10 aa, Homo sapiens
  MOD_RES (8)..(8): Phosphorylated Tyrosine
PIEDVTIYEK

SEQ ID NO: 126 - protein, 8 aa, Homo sapiens
  MOD_RES (4)..(4): Phosphorylated Tyrosine
ITGYIVEK

SEQ ID NO: 127 - protein, 22 aa, Homo sapiens
  MOD_RES (15)..(15): Phosphorylated Tyrosine
TLELCVDNLQPDFLYDHIQPVR

SEQ ID NO: 128 - protein, 21 aa, Homo sapiens
  MOD_RES (7)..(7): Phosphorylated Tyrosine
GHNGKIYPYLVMNDACLTESR

SEQ ID NO: 129 - protein, 10 aa, Homo sapiens
  MOD_RES (3)..(3): Phosphorylated Tyrosine
DDYGFSGSQK

SEQ ID NO: 130 - protein, 11 aa, Homo sapiens
  MOD_RES (10)..(10): Phosphorylated Tyrosine
IGKGSFGEVYK

SEQ ID NO: 131 - protein, 24 aa, Homo sapiens
  MOD_RES (8)..(8): Phosphorylated Tyrosine
NGYIPSNYVAPADSIQAEEWYFGK

SEQ ID NO: 132 - protein, 11 aa, Homo sapiens
  MOD_RES (3)..(3): Phosphorylated Tyrosine
KLYSGAQTDGK

SEQ ID NO: 133 - protein, 14 aa, Homo sapiens
  MOD_RES (12)..(12): Phosphorylated Tyrosine
KEQGTYALSLIYGK

SEQ ID NO: 134 - protein, 11 aa, Homo sapiens
  MOD_RES (5)..(5): Phosphorylated Tyrosine
MRACYYSLASK

SEQ ID NO: 135 - protein, 11 aa, Homo sapiens
  MOD_RES (6)..(6): Phosphorylated Tyrosine
MRACYYSLASK

SEQ ID NO: 136 - protein, 11 aa, Homo sapiens
  MOD_RES (7)..(7): Phosphorylated Tyrosine
NCDDEVYELMR

SEQ ID NO: 137 - protein, 15 aa, Homo sapiens
  MOD_RES (7)..(7): Phosphorylated Tyrosine
VFLAECYNLSPTKDK

SEQ ID NO: 138 - protein, 12 aa, Homo sapiens
  MOD_RES (3)..(3): Phosphorylated Tyrosine
EVYDVMLGCWQR

SEQ ID NO: 139 - protein, 19 aa, Homo sapiens
  MOD_RES (7)..(7): Phosphorylated Tyrosine
KIYSGDYYRQGCASKLPVK

SEQ ID NO: 140 - protein, 19 aa, Homo sapiens
  MOD_RES (8)..(8): Phosphorylated Tyrosine
KIYSGDYYRQGCASKLPVK

SEQ ID NO: 141 - protein, 10 aa, Homo sapiens
  MOD_RES (8)..(8): Phosphorylated Tyrosine
LVGDTVSYSK

SEQ ID NO: 142 - protein, 32 aa, Homo sapiens
  MOD_RES (26)..(26): Phosphorylated Tyrosine
NIFVAWFVTVPVAGLFSAAVMALLMYGILPYV

SEQ ID NO: 143 - protein, 32 aa, Homo sapiens; identical to SEQ ID NO: 142
  MOD_RES (31)..(31): Phosphorylated Tyrosine

SEQ ID NO: 144 - protein, 12 aa, Homo sapiens
  MOD_RES (11)..(11): Phosphorylated Tyrosine
GSGSVVGELMYK

SEQ ID NO: 145 - protein, 25 aa, Homo sapiens
  MOD_RES (22)..(22): Phosphorylated Tyrosine
FSISPDEDSSSYSSNSDFNYSYPTK

SEQ ID NO: 146 - protein, 12 aa, Homo sapiens
  MOD_RES (3)..(3): Phosphorylated Tyrosine
AVYIGVHVPFSK

SEQ ID NO: 147 - protein, 18 aa, Homo sapiens
  MOD_RES (6)..(6): Phosphorylated Tyrosine
IGSSLYALGTQDSTDICK

SEQ ID NO: 148 - protein, 33 aa, Homo sapiens
  MOD_RES (23)..(23): Phosphorylated Tyrosine
SNVIVALARDSLALARPKSSDVYVSYDYGKSFK

SEQ ID NO: 149 - protein, 33 aa, Homo sapiens; identical to SEQ ID NO: 148
  MOD_RES (26)..(26): Phosphorylated Tyrosine

SEQ ID NO: 150 - protein, 29 aa, Homo sapiens
  MOD_RES (7)..(7): Phosphorylated Tyrosine
REVAPPYQGADPILATALASDPIPNPLQK

SEQ ID NO: 151 - protein, 30 aa, Homo sapiens
  MOD_RES (20)..(20): Phosphorylated Tyrosine
WEDSAHKPQSLDTDDPATLYAVVENVPPLR

SEQ ID NO: 152 - protein, 19 aa, Homo sapiens
  MOD_RES (19)..(19): Phosphorylated Tyrosine
INESLRDQLLVTIQKTFTY

SEQ ID NO: 153 - protein, 21 aa, Homo sapiens
  MOD_RES (15)..(15): Phosphorylated Tyrosine
ASLRDNLYLLQAFMYILGICL

SEQ ID NO: 154 - protein, 23 aa, Homo sapiens
  MOD_RES (3)..(3): Phosphorylated Tyrosine
PPYYPQPENPDSGGNIYPRPKPR

SEQ ID NO: 155 - protein, 19 aa, Homo sapiens
  MOD_RES (9)..(9): Phosphorylated Tyrosine
KRYDQDLCYTDILFTEQER

SEQ ID NO: 156 - protein, 8 aa, Homo sapiens
  MOD_RES (7)..(7): Phosphorylated Tyrosine
MLQHIDYR

SEQ ID NO: 157 - protein, 19 aa, Homo sapiens
  MOD_RES (9)..(9): Phosphorylated Tyrosine
AAFGISDSYVDGSSFDPQR

SEQ ID NO: 158 - protein, 21 aa, Homo sapiens
  MOD_RES (8)..(8): Phosphorylated Tyrosine
FQSDSSSYPTVDSNSLLGQSR

SEQ ID NO: 159 - protein, 8 aa, Homo sapiens
  MOD_RES (6)..(6): Phosphorylated Tyrosine
VPLSAYER

SEQ ID NO: 160 - protein, 27 aa, Homo sapiens
  MOD_RES (3)..(3): Phosphorylated Tyrosine
SPYSRSRYRESRYGGSHYSSSGYSNSR

SEQ ID NO: 161 - protein, 27 aa, Homo sapiens
  MOD_RES (8)..(8): Phosphorylated Tyrosine
SPYSRSRYRESRYGGSHYSSSGYSNSR

SEQ ID NO: 162 - protein, 27 aa, Homo sapiens
  MOD_RES (18)..(18): Phosphorylated Tyrosine
SPYSRSRYRESRYGGSHYSSSGYSNSR

SEQ ID NO: 163 - protein, 17 aa, Homo sapiens
  MOD_RES (10)..(10): Phosphorylated Tyrosine
GILHLSPDVYQEMEASR

SEQ ID NO: 164 - protein, 15 aa, Homo sapiens
  MOD_RES (7)..(7): Phosphorylated Tyrosine
SQSYGGYENQKQSSY

SEQ ID NO: 165 - protein, 29 aa, Homo sapiens
  MOD_RES (11)..(11): Phosphorylated Tyrosine
GGYGGDRGGGYGGDRGGYGGDRGGYGGDR

SEQ ID NO: 166 - protein, 9 aa, Homo sapiens
  MOD_RES (4)..(4): Phosphorylated Tyrosine
FTEYETQVK

SEQ ID NO: 167 - protein, 29 aa, Homo sapiens
  MOD_RES (10)..(10): Phosphorylated Tyrosine
IHAYVDYPIYDVLQMVGHANRPLQDDEGR

SEQ ID NO: 168 - protein, 17 aa, Homo sapiens
  MOD_RES (8)..(8): Phosphorylated Tyrosine
RMTQNPNYYNLQGISHR

SEQ ID NO: 169 - protein, 28 aa, Homo sapiens
  MOD_RES (18)..(18): Phosphorylated Tyrosine
KSLEVWTKDHPFPIDIDYMISDTLELLR

SEQ ID NO: 170 - protein, 11 aa, Homo sapiens
  MOD_RES (10)..(10): Phosphorylated Tyrosine
VGTIDDDPEYR

SEQ ID NO: 171 - protein, 15 aa, Homo sapiens
  MOD_RES (10)..(10): Phosphorylated Tyrosine
NKDRPAMQLYQPGAR

SEQ ID NO: 172 - protein, 10 aa, Homo sapiens
  MOD_RES (3)..(3): Phosphorylated Tyrosine
TGYGGGFNER

SEQ ID NO: 173 - protein, 21 aa, Homo sapiens
  MOD_RES (5)..(5): Phosphorylated Tyrosine
ENVEYIEREESDGEYDEFGRK

SEQ ID NO: 174 - protein, 21 aa, Homo sapiens
  MOD_RES (15)..(15): Phosphorylated Tyrosine
ENVEYIEREESDGEYDEFGRK

SEQ ID NO: 175 - protein, 15 aa, Homo sapiens
  MOD_RES (11)..(11): Phosphorylated Tyrosine
AYHNSPAYLAYINAK

SEQ ID NO: 176 - protein, 9 aa, Homo sapiens
  MOD_RES (4)..(4): Phosphorylated Tyrosine
SAVYPLLYR

SEQ ID NO: 177 - protein, 33 aa, Homo sapiens
  MOD_RES (11)..(11): Phosphorylated Tyrosine
TPQGREYGMIYLGKDTNGENIAESLVAEGLATR

SEQ ID NO: 178 - protein, 13 aa, Homo sapiens
  MOD_RES (10)..(10): Phosphorylated Tyrosine
DQREEELGEYYMK

SEQ ID NO: 179 - protein, 23 aa, Homo sapiens
  MOD_RES (12)..(12): Phosphorylated Tyrosine
GIYKDDIAQVDYVEPSQNTISLK

SEQ ID NO: 180 - protein, 16 aa, Homo sapiens
  MOD_RES (11)..(11): Phosphorylated Tyrosine
TPMYGSQTPMYGSGSR

SEQ ID NO: 181 - protein, 15 aa, Homo sapiens
  MOD_RES (4)..(4): Phosphorylated Tyrosine
TPMYGSQTPLQDGSR

SEQ ID NO: 182 - protein, 24 aa, Homo sapiens
  MOD_RES (1)..(1): Phosphorylated Tyrosine
YPDGHGDYAYQQSSYTEQSYDRSF

SEQ ID NO: 183 - protein, 24 aa, Homo sapiens
  MOD_RES (10)..(10): Phosphorylated Tyrosine
YPDGHGDYAYQQSSYTEQSYDRSF

SEQ ID NO: 184 - protein, 24 aa, Homo sapiens
  MOD_RES (15)..(15): Phosphorylated Tyrosine
YPDGHGDYAYQQSSYTEQSYDRSF

SEQ ID NO: 185 - protein, 16 aa, Homo sapiens
  MOD_RES (7)..(7): Phosphorylated Tyrosine
ICHQIEYYFGDFNLPR

SEQ ID NO: 186 - protein, 24 aa, Homo sapiens
  MOD_RES (21)..(21): Phosphorylated Tyrosine
EGMNPSYDEYADSDEDQHDAYLER

SEQ ID NO: 187 - protein, 26 aa, Homo sapiens
  MOD_RES (19)..(19): Phosphorylated Tyrosine
RNLQDNFQEDPIQMSMIIYSCLKEER

SEQ ID NO: 188 - protein, 14 aa, Homo sapiens
  MOD_RES (3)..(3): Phosphorylated Tyrosine
YLYPNIDKDHAFGK

SEQ ID NO: 189 - protein, 18 aa, Homo sapiens
  MOD_RES (14)..(14): Phosphorylated Tyrosine
QFLAPWIESQDWAYAASK

SEQ ID NO: 190 - protein, 27 aa, Homo sapiens
  MOD_RES (16)..(16): Phosphorylated Tyrosine
IMDATNILVSPLVYLYPDIPKEEAFGK

SEQ ID NO: 191 - protein, 22 aa, Homo sapiens
  MOD_RES (1)..(1): Phosphorylated Tyrosine
YCRPESQEHPEADPGSAAPYLK

SEQ ID NO: 192 - protein, 18 aa, Homo sapiens
  MOD_RES (8)..(8): Phosphorylated Tyrosine
KLQQTQEYFIIQYQESLR

SEQ ID NO: 193 - protein, 13 aa, Homo sapiens
  MOD_RES (2)..(2): Phosphorylated Tyrosine
YYTPVPCESATAK

SEQ ID NO: 194 - protein, 16 aa, Homo sapiens
  MOD_RES (1)..(1): Phosphorylated Tyrosine
YALAVRQDVINTLLPK

SEQ ID NO: 195 - protein, 20 aa, Homo sapiens
  MOD_RES (15)..(15): Phosphorylated Tyrosine
RAEALQSIATDPGLYQMLPR

SEQ ID NO: 196 - protein, 31 aa, Homo sapiens
  MOD_RES (24)..(24): Phosphorylated Tyrosine
DGDTQTDAGGEPDSLGQQPTDTPYEWDLDKK

SEQ ID NO: 197 - protein, 18 aa, Homo sapiens
  MOD_RES (16)..(16): Phosphorylated Tyrosine
EFDEDSDEKEEEEDTYEK

SEQ ID NO: 198 - protein, 18 aa, Homo sapiens
  MOD_RES (14)..(14): Phosphorylated Tyrosine
VFDDESDEKEDEEYADEK

SEQ ID NO: 199 - protein, 14 aa, Homo sapiens
  MOD_RES (8)..(8): Phosphorylated Tyrosine
TPDGKVYYYNARTR

SEQ ID NO: 200 - protein, 14 aa, Homo sapiens
  MOD_RES (9)..(9): Phosphorylated Tyrosine
TPDGKVYYYNARTR

SEQ ID NO: 201 - protein, 16 aa, Homo sapiens
  MOD_RES (8)..(8): Phosphorylated Tyrosine
GFTDSPHYSDHLNDSR

SEQ ID NO: 202 - protein, 30 aa, Homo sapiens
  MOD_RES (21)..(21): Phosphorylated Tyrosine
QDLGLGSPAQLSSSGKPGTAYYSFSATSSR

SEQ ID NO: 203 - protein, 30 aa, Homo sapiens; identical to SEQ ID NO: 202
  MOD_RES (22)..(22): Phosphorylated Tyrosine

SEQ ID NO: 204 - protein, 11 aa, Homo sapiens
  MOD_RES (7)..(7): Phosphorylated Tyrosine
QINLTDYPIPR

SEQ ID NO: 205 - protein, 24 aa, Homo sapiens
  MOD_RES (10)..(10): Phosphorylated Tyrosine
QVVEKVETEYARYENGHYSYRIHR

SEQ ID NO: 206 - protein, 24 aa, Homo sapiens
  MOD_RES (13)..(13): Phosphorylated Tyrosine
QVVEKVETEYARYENGHYSYRIHR

SEQ ID NO: 207 - protein, 24 aa, Homo sapiens
  MOD_RES (18)..(18): Phosphorylated Tyrosine
QVVEKVETEYARYENGHYSYRIHR

SEQ ID NO: 208 - protein, 16 aa, Homo sapiens
  MOD_RES (5)..(5): Phosphorylated Tyrosine
LQPIYWSRDDVAQWLK

SEQ ID NO: 209 - protein, 19 aa, Homo sapiens
  MOD_RES (11)..(11): Phosphorylated Tyrosine
YRSPHSGDVLYELLQHILK

SEQ ID NO: 210 - protein, 14 aa, Homo sapiens
  MOD_RES (7)..(7): Phosphorylated Tyrosine
LLWDYVYQLLSDSR

SEQ ID NO: 211 - protein, 9 aa, Homo sapiens
  MOD_RES (2)..(2): Phosphorylated Tyrosine
HYYKLNIIR

SEQ ID NO: 212 - protein, 9 aa, Homo sapiens
  MOD_RES (3)..(3): Phosphorylated Tyrosine
HYYKLNIIR

SEQ ID NO: 213 - protein, 13 aa, Homo sapiens
  MOD_RES (11)..(11): Phosphorylated Tyrosine
AECRPAASENYMR

SEQ ID NO: 214 - protein, 19 aa, Homo sapiens
  MOD_RES (17)..(17): Phosphorylated Tyrosine
EDLQLDKPASGVKEEWYAR

SEQ ID NO: 215 - protein, 21 aa, Homo sapiens
  MOD_RES (6)..(6): Phosphorylated Tyrosine
LPPYPYSSPSLVLPTQPHTPK

SEQ ID NO: 216 - protein, 20 aa, Homo sapiens
  MOD_RES (13)..(13): Phosphorylated Tyrosine
ASESSKPWPDATYGTGSASR

SEQ ID NO: 217 - protein, 11 aa, Homo sapiens
  MOD_RES (4)..(4): Phosphorylated Tyrosine
DGAYSLFDNSK

SEQ ID NO: 218 - protein, 14 aa, Homo sapiens
  MOD_RES (12)..(12): Phosphorylated Tyrosine
SSGFAFDPSVNYSK

SEQ ID NO: 219 - protein, 9 aa, Homo sapiens
  MOD_RES (5)..(5): Phosphorylated Tyrosine
LNNEYEVIK

SEQ ID NO: 220 - protein, 18 aa, Homo sapiens
  MOD_RES (5)..(5): Phosphorylated Tyrosine
LQVDYTGLIQSYEQNETK

SEQ ID NO: 221 - protein, 8 aa, Homo sapiens
  MOD_RES (5)..(5): Phosphorylated Tyrosine
SHLMYAVR

SEQ ID NO: 222 - protein, 9 aa, Homo sapiens
  MOD_RES (4)..(4): Phosphorylated Tyrosine
DKVYEQLAK

SEQ ID NO: 223 - protein, 28 aa, Homo sapiens
  MOD_RES (21)..(21): Phosphorylated Tyrosine
DIDHETVVEEQIIGENSPPDYSEYMTGK

SEQ ID NO: 224 - protein, 28 aa, Homo sapiens
  MOD_RES (24)..(24): Phosphorylated Tyrosine
DIDHETVVEEQIIGENSPPDYSEYMTGK

SEQ ID NO: 225 - protein, 12 aa, Homo sapiens
  MOD_RES (5)..(5): Phosphorylated Tyrosine
HLGIYSVLPNHK

SEQ ID NO: 226 - protein, 12 aa, Homo sapiens
  MOD_RES (9)..(9): Phosphorylated Tyrosine
KIHSADKPYKCK

SEQ ID NO: 227 - protein, 10 aa, Homo sapiens
  MOD_RES (7)..(7): Phosphorylated Tyrosine
CDTCQQYFSR

SEQ ID NO: 228 - protein, 21 aa, Homo sapiens
  MOD_RES (7)..(7): Phosphorylated Tyrosine
KNTDKNYLNFVSPLPDIVGQK

SEQ ID NO: 229 - protein, 11 aa, Homo sapiens
  MOD_RES (3)..(3): Phosphorylated Tyrosine
LAYQELQIDRK

SEQ ID NO: 230 - protein, 19 aa, Homo sapiens
  MOD_RES (7)..(7): Phosphorylated Tyrosine
DVMLENYSNLVSVGYCVHK

SEQ ID NO: 231 - protein, 9 aa, Homo sapiens
  MOD_RES (3)..(3): Phosphorylated Tyrosine
TGYSFVNCK

SEQ ID NO: 232 - protein, 19 aa, Homo sapiens
  MOD_RES (14)..(14): Phosphorylated Tyrosine
DLEKPFLLPVEAVYSVPGR

SEQ ID NO: 233 - protein, 11 aa, Homo sapiens
  MOD_RES (8)..(8): Phosphorylated Tyrosine
VPTTLAEYCIK

SEQ ID NO: 234 - protein, 17 aa, Homo sapiens
  MOD_RES (8)..(8): Phosphorylated Tyrosine
QQQQEGNYLQHVQLDQR

SEQ ID NO: 235 - protein, 9 aa, Homo sapiens
  MOD_RES (7)..(7): Phosphorylated Tyrosine
LLDEELYSR

SEQ ID NO: 236 - protein, 16 aa, Homo sapiens
  MOD_RES (12)..(12): Phosphorylated Tyrosine
ALSNLESIPGGYNALR

SEQ ID NO: 237 - protein, 13 aa, Homo sapiens
  MOD_RES (5)..(5): Phosphorylated Tyrosine
CCLTYCFNKPEDK

SEQ ID NO: 238 - protein, 18 aa, Homo sapiens
  MOD_RES (1)..(1): Phosphorylated Tyrosine
YTVQFTTMVQVNEETGNR

SEQ ID NO: 239 - protein, 36 aa, Homo sapiens
  MOD_RES (15)..(15): Phosphorylated Tyrosine
SVPVSAGGEGETSPYSLEASPLGQLMNMLSHPVIRR

SEQ ID NO: 240 - protein, 11 aa, Homo sapiens
  MOD_RES (7)..(7): Phosphorylated Tyrosine
EMFNPMYALFR

SEQ ID NO: 241 - protein, 30 aa, Homo sapiens
  MOD_RES (28)..(28): Phosphorylated Tyrosine
SSVELPPYSGTVLCGTQAVDKLPDGQEYQR

SEQ ID NO: 242 - protein, 13 aa, Homo sapiens
  MOD_RES (6)..(6): Phosphorylated Tyrosine
SQFLGYQQHDSQE

SEQ ID NO: 243 - protein, 29 aa, Homo sapiens
  MOD_RES (17)..(17): Phosphorylated Tyrosine
YEPFSFADDIGSNNCGYYDLQAVLTHQGR

SEQ ID NO: 244 - protein, 13 aa, Homo sapiens
  MOD_RES (9)..(9): Phosphorylated Tyrosine
NSNYCLPSYTAYK

SEQ ID NO: 245 - protein, 12 aa, Homo sapiens
  MOD_RES (2)..(2): Phosphorylated Tyrosine
GYAQQDTQEFLR

SEQ ID NO: 246 - protein, 11 aa, Homo sapiens
  MOD_RES (6)..(6): Phosphorylated Tyrosine
YMSNTYEQLSK

SEQ ID NO: 247 - protein, 19 aa, Homo sapiens
  MOD_RES (10)..(10): Phosphorylated Tyrosine
VSVTFDPFCYLTLPLPLKK

SEQ ID NO: 248 - protein, 9 aa, Homo sapiens
  MOD_RES (2)..(2): Phosphorylated Tyrosine
NYEQWQSQR

SEQ ID NO: 249 - protein, 11 aa, Homo sapiens
  MOD_RES (8)..(8): Phosphorylated Tyrosine
AEVENKGYSYK

SEQ ID NO: 250 - protein, 12 aa, Homo sapiens
  MOD_RES (4)..(4): Phosphorylated Tyrosine
SPAYSVSTIEPR

SEQ ID NO: 251 - protein, 19 aa, Homo sapiens
  MOD_RES (10)..(10): Phosphorylated Tyrosine
SIDDQSQQSYDYGGSGGPY

SEQ ID NO: 252 - protein, 19 aa, Homo sapiens
  MOD_RES (12)..(12): Phosphorylated Tyrosine
SIDDQSQQSYDYGGSGGPY

SEQ ID NO: 253 - protein, 11 aa, Homo sapiens
  MOD_RES (5)..(5): Phosphorylated Tyrosine
TGQIYQPTQAY

SEQ ID NO: 254 - protein, 21 aa, Homo sapiens
  MOD_RES (18)..(18): Phosphorylated Tyrosine
YSKEEEMDDMDRDLGDEYGWK

SEQ ID NO: 255 - protein, 14 aa, Homo sapiens
  MOD_RES (10)..(10): Phosphorylated Tyrosine
SLKIEAENKYDAIK

SEQ ID NO: 256 - protein, 14 aa, Homo sapiens
  MOD_RES (12)..(12): Phosphorylated Tyrosine
EKLALALENEGYIK

SEQ ID NO: 257 - protein, 10 aa, Homo sapiens
  MOD_RES (3)..(3): Phosphorylated Tyrosine
NGYVTVSEIK

SEQ ID NO: 258 - protein, 13 aa, Homo sapiens
  MOD_RES (10)..(10): Phosphorylated Tyrosine
SLHEELQKIYKTY

SEQ ID NO: 259 - protein, 12 aa, Homo sapiens
  MOD_RES (5)..(5): Phosphorylated Tyrosine
SVDAYDSYWESR

SEQ ID NO: 260 - protein, 12 aa, Homo sapiens
  MOD_RES (8)..(8): Phosphorylated Tyrosine
SVDAYDSYWESR

SEQ ID NO: 261 - protein, 18 aa, Homo sapiens
  MOD_RES (9)..(9): Phosphorylated Tyrosine
IVLPGNFLYCTFYGRPYK

SEQ ID NO: 262 - protein, 13 aa, Homo sapiens
  MOD_RES (3)..(3): Phosphorylated Tyrosine
IIYVPLPDAATRR

SEQ ID NO: 263 - protein, 12 aa, Homo sapiens
  MOD_RES (7)..(7): Phosphorylated Tyrosine
SSEKIIYVYMKR

SEQ ID NO: 264 - protein, 12 aa, Homo sapiens
  MOD_RES (9)..(9): Phosphorylated Tyrosine
SSEKIIYVYMKR

SEQ ID NO: 265 - protein, 17 aa, Homo sapiens
  MOD_RES (8)..(8): Phosphorylated Tyrosine
MDVVFGKYWLSQCFDAK

SEQ ID NO: 266 - protein, 15 aa, Homo sapiens
  MOD_RES (5)..(5): Phosphorylated Tyrosine
DFGNYLFNFASAATK

SEQ ID NO: 267 - protein, 18 aa, Homo sapiens
  MOD_RES (9)..(9): Phosphorylated Tyrosine
ELQQELQEYEVVTESEKR

SEQ ID NO: 268 - protein, 16 aa, Homo sapiens
  MOD_RES (14)..(14): Phosphorylated Tyrosine
MSGRAAATTPTKVYSE

SEQ ID NO: 269 - protein, 20 aa, Homo sapiens
  MOD_RES (2)..(2): Phosphorylated Tyrosine
LYEDILLQMDFIHIAQFLTK

SEQ ID NO: 270 - protein, 20 aa, Homo sapiens
  MOD_RES (4)..(4): Phosphorylated Tyrosine
ATDYPCLLILDPQNEFETLR

SEQ ID NO: 271 - protein, 13 aa, Homo sapiens
  MOD_RES (8)..(8): Phosphorylated Tyrosine
HFNVNTDYQNPVR

SEQ ID NO: 272 - protein, 13 aa, Homo sapiens
  MOD_RES (9)..(9): Phosphorylated Tyrosine
STRPTQQFYQPPR

SEQ ID NO: 273 - protein, 15 aa, Homo sapiens
  MOD_RES (8)..(8): Phosphorylated Tyrosine
IHTGEKPYTCPDCGR

SEQ ID NO: 274 - protein, 12 aa, Homo sapiens
  MOD_RES (9)..(9): Phosphorylated Tyrosine
LYHEADKTYMLR

SEQ ID NO: 275 - protein, 14 aa, Homo sapiens
  MOD_RES (4)..(4): Phosphorylated Tyrosine
VNKYVNLVMYFLFR

SEQ ID NO: 276 - protein, 14 aa, Homo sapiens
  MOD_RES (10)..(10): Phosphorylated Tyrosine
VNKYVNLVMYFLFR

SEQ ID NO: 277 - protein, 21 aa, Homo sapiens
  MOD_RES (6)..(6): Phosphorylated Tyrosine
DSQGTYSSRDAELQDQEFGKR

SEQ ID NO: 278 - protein, 10 aa, Homo sapiens
  MOD_RES (7)..(7): Phosphorylated Tyrosine
DVSLGTYGSR

SEQ ID NO: 279 - protein, 11 aa, Homo sapiens
  MOD_RES (2)..(2): Phosphorylated Tyrosine
SYQFGIIGNDR

SEQ ID NO: 280 - protein, 28 aa, Homo sapiens
  MOD_RES (10)..(10): Phosphorylated Tyrosine
DMGTTDSGPYFEKGGSHGLFGNSTAQSR

SEQ ID NO: 281 - protein, 36 aa, Homo sapiens
  MOD_RES (12)..(12): Phosphorylated Tyrosine
GIQNIDPESDPYVTPGSVLGGTATSPIVDTDHQLLR

SEQ ID NO: 282 - protein, 17 aa, Homo sapiens
  MOD_RES (1)..(1): Phosphorylated Tyrosine
YEAPQATDGLAGALDAR

SEQ ID NO: 283 - protein, 12 aa, Homo sapiens
  MOD_RES (11)..(11): Phosphorylated Tyrosine
GWQDVTATSAYK

SEQ ID NO: 284 - protein, 13 aa, Homo sapiens
  MOD_RES (11)..(11): Phosphorylated Tyrosine
SWHDVQVSSAYVK

SEQ ID NO: 285 - protein, 14 aa, Homo sapiens
  MOD_RES (9)..(9): Phosphorylated Tyrosine
EYEKAKKTYMQACK

SEQ ID NO: 286 - protein, 29 aa, Homo sapiens
  MOD_RES (6)..(6): Phosphorylated Tyrosine
EQFLQYAYDITFDPDTAHKYLRLQEENRK

SEQ ID NO: 287 - protein, 17 aa, Homo sapiens
  MOD_RES (1)..(1): Phosphorylated Tyrosine
YRPLFGYWVIGLQNKCK

SEQ ID NO: 288 - protein, 17 aa, Homo sapiens
  MOD_RES (7)..(7): Phosphorylated Tyrosine
YRPLFGYWVIGLQNKCK

SEQ ID NO: 289 - protein, 18 aa, Homo sapiens
  MOD_RES (6)..(6): Phosphorylated Tyrosine
TGVSPYSKTLVLQTSEGK

SEQ ID NO: 290 - protein, 11 aa, Homo sapiens
  MOD_RES (7)..(7): Phosphorylated Tyrosine
IQHNSDYSYPK

SEQ ID NO: 291 - protein, 11 aa, Homo sapiens
  MOD_RES (9)..(9): Phosphorylated Tyrosine
IQHNSDYSYPK

SEQ ID NO: 292 - protein, 12 aa, Homo sapiens
  MOD_RES (4)..(4): Phosphorylated Tyrosine
ATDYGPEEVCER

SEQ ID NO: 293 - protein, 11 aa, Homo sapiens
  MOD_RES (5)..(5): Phosphorylated Tyrosine
GKEPYTKKALK

SEQ ID NO: 294 - protein, 26 aa, Homo sapiens
  MOD_RES (14)..(14): Phosphorylated Tyrosine
FSTQGMGTFNPADYSDSTSTDVCGTK

SEQ ID NO: 295 - protein, 16 aa, Homo sapiens
  MOD_RES (8)..(8): Phosphorylated Tyrosine
SQASKPAYGNSPYWTN

SEQ ID NO: 296 - protein, 16 aa, Homo sapiens
  MOD_RES (13)..(13): Phosphorylated Tyrosine
SQASKPAYGNSPYWTN

SEQ ID NO: 297 - protein, 14 aa, Homo sapiens
  MOD_RES (9)..(9): Phosphorylated Tyrosine
HLSEKIANYKEGTY

SEQ ID NO: 298 - protein, 23 aa, Homo sapiens
  MOD_RES (10)..(10): Phosphorylated Tyrosine
EGSVGSTSDYVSQSYSYSSILNK

SEQ ID NO: 299 - protein, 23 aa, Homo sapiens
  MOD_RES (15)..(15): Phosphorylated Tyrosine
EGSVGSTSDYVSQSYSYSSILNK

SEQ ID NO: 300 - protein, 26 aa, Homo sapiens
  MOD_RES (6)..(6): Phosphorylated Tyrosine
PPSSPYPPLPKDKLNPPTPSIYLENK

SEQ ID NO: 301 - protein, 11 aa, Homo sapiens
  MOD_RES (5)..(5): Phosphorylated Tyrosine
LHFTYENLNSK

SEQ ID NO: 302 - protein, 34 aa, Homo sapiens
  MOD_RES (32)..(32): Phosphorylated Tyrosine
KIKNAHSLAHSEETSAMSNTMVNKDDLPVSKYYR

SEQ ID NO: 303 - protein, 34 aa, Homo sapiens; identical to SEQ ID NO: 302
  MOD_RES (33)..(33): Phosphorylated Tyrosine

SEQ ID NO: 304 - protein, 15 aa, Homo sapiens
  MOD_RES (8)..(8): Phosphorylated Tyrosine
VHTSGFGYQSELELR

SEQ ID NO: 305 - protein, 14 aa, Homo sapiens
  MOD_RES (7)..(7): Phosphorylated Tyrosine
REDKELYTVVQHVK

SEQ ID NO: 306 - protein, 36 aa, Homo sapiens
  MOD_RES (9)..(9): Phosphorylated Tyrosine
AAEAAASAYYNPGNPHNVYMPTSQPPPPPYYPPEDK

SEQ ID NO: 307 - protein, 11 aa, Homo sapiens
  MOD_RES (3)..(3): Phosphorylated Tyrosine
QLYALVCETQR

SEQ ID NO: 308 - protein, 10 aa, Homo sapiens
  MOD_RES (2)..(2): Phosphorylated Tyrosine
NYISAWELQR

SEQ ID NO: 309 - protein, 17 aa, Homo sapiens
  MOD_RES (6)..(6): Phosphorylated Tyrosine
TMTCEYALCSFFVPGDR

SEQ ID NO: 310 - protein, 17 aa, Homo sapiens
  MOD_RES (7)..(7): Phosphorylated Tyrosine
MSLLLVYGSWFQPTIER

SEQ ID NO: 311 - protein, 15 aa, Homo sapiens
  MOD_RES (7)..(7): Phosphorylated Tyrosine
AAEDSPYWVSPAYSK

SEQ ID NO: 312 - protein, 16 aa, Homo sapiens
  MOD_RES (2)..(2): Phosphorylated Tyrosine
PYEKIPDQMFFHTDYR

SEQ ID NO: 313 - protein, 13 aa, Homo sapiens
  MOD_RES (2)..(2): Phosphorylated Tyrosine
KYPLTVFESRGFR

SEQ ID NO: 314 - protein, 20 aa, Homo sapiens
  MOD_RES (9)..(9): Phosphorylated Tyrosine
TIKETDPDYEDVSVALPNKR

SEQ ID NO: 315 - protein, 11 aa, Homo sapiens
  MOD_RES (10)..(10): Phosphorylated Tyrosine
YGLLPSHASYL

SEQ ID NO: 316 - protein, 12 aa, Homo sapiens
  MOD_RES (11)..(11): Phosphorylated Tyrosine
YSDVHNCSYNYK

SEQ ID NO: 317 - protein, 9 aa, Homo sapiens
  MOD_RES (7)..(7): Phosphorylated Tyrosine
SVNEGAYIR

SEQ ID NO: 318 - protein, 12 aa, Homo sapiens
  MOD_RES (8)..(8): Phosphorylated Tyrosine
VHTGEKPYACLK

SEQ ID NO: 319 - protein, 30 aa, Homo sapiens
  MOD_RES (2)..(2): Phosphorylated Tyrosine
RYWQNLEANVPGAQVLGNQIMPGFLNMKIK

SEQ ID NO: 320 - protein, 16 aa, Homo sapiens
  MOD_RES (5)..(5): Phosphorylated Tyrosine
LMKNYSQPMPLFDRSK

SEQ ID NO: 321 - protein, 9 aa, Homo sapiens
  MOD_RES (4)..(4): Phosphorylated Tyrosine
ATQYGIFER

SEQ ID NO: 322 - protein, 21 aa, Homo sapiens
  MOD_RES (1)..(1): Phosphorylated Tyrosine
YRPGYSSSSTSAAMPHSSSAK

SEQ ID NO: 323 - protein, 21 aa, Homo sapiens
  MOD_RES (5)..(5): Phosphorylated Tyrosine
YRPGYSSSSTSAAMPHSSSAK

SEQ ID NO: 324 - protein, 16 aa, Homo sapiens
  MOD_RES (13)..(13): Phosphorylated Tyrosine
GVGSPEPGPTAPYLGR

SEQ ID NO: 325 - protein, 37 aa, Homo sapiens
  MOD_RES (13)..(13): Phosphorylated Tyrosine
SQADSLFGDSGVYDAPSSYSLQQASVLSEGPRGPALR

SEQ ID NO: 326 - protein, 16 aa, Homo sapiens
  MOD_RES (12)..(12): Phosphorylated Tyrosine
KYDIYFADGKVYALYR

SEQ ID NO: 327 - protein, 11 aa, Homo sapiens
  MOD_RES (8)..(8): Phosphorylated Tyrosine
HIDLQFSYAPR

SEQ ID NO: 328 - protein, 19 aa, Homo sapiens
  MOD_RES (17)..(17): Phosphorylated Tyrosine
RNEGVVGGEDYEEVDRYSR

SEQ ID NO: 329 - protein, 37 aa, Homo sapiens
  MOD_RES (32)..(32): Phosphorylated Tyrosine
LKDEDFPSLSASTSSSCSTAATPGPVGLALPYAIPAR

SEQ ID NO: 330 - protein, 18 aa, Homo sapiens
  MOD_RES (17)..(17): Phosphorylated Tyrosine
DDDRTPGLHGDCDDDKYR

SEQ ID NO: 331 - protein, 19 aa, Homo sapiens
  MOD_RES (6)..(6): Phosphorylated Tyrosine
RHRLQYKKKVNPDLQVEVK

SEQ ID NO: 332 - protein, 9 aa, Homo sapiens
  MOD_RES (7)..(7): Phosphorylated Tyrosine
RYEEDMYWR

SEQ ID NO: 333 - protein, 15 aa, Homo sapiens
  MOD_RES (8)..(8): Phosphorylated Tyrosine
THSGEKPYECSYCGK

SEQ ID NO: 334 - protein, 24 aa, Homo sapiens
  MOD_RES (16)..(16): Phosphorylated Tyrosine
SAKPVGPEDMGATAVYELDTEKER

SEQ ID NO: 335 - protein, 9 aa, Homo sapiens
  MOD_RES (8)..(8): Phosphorylated Tyrosine
GKEDDKIYR

SEQ ID NO: 336 - protein, 11 aa, Homo sapiens
  MOD_RES (10)..(10): Phosphorylated Tyrosine
FCCQSCVSEYK

SEQ ID NO: 337 - protein, 27 aa, Homo sapiens
  MOD_RES (13)..(13): Phosphorylated Tyrosine
FFDMTQCIGPNGYMEPYCSTACMNSHK

SEQ ID NO: 338 - protein, 26 aa, Homo sapiens
  MOD_RES (6)..(6): Phosphorylated Tyrosine
NSLPQYQATMPDGKLYNFCNSSCVAK

SEQ ID NO: 339 - protein, 26 aa, Homo sapiens
  MOD_RES (16)..(16): Phosphorylated Tyrosine
NSLPQYQATMPDGKLYNFCNSSCVAK

SEQ ID NO: 340 - protein, 23 aa, Homo sapiens
  MOD_RES (6)..(6): Phosphorylated Tyrosine
TCSDDYKKLHCIVTYCEYCQEEK

SEQ ID NO: 341 - protein, 23 aa, Homo sapiens
  MOD_RES (15)..(15): Phosphorylated Tyrosine
TCSDDYKKLHCIVTYCEYCQEEK

SEQ ID NO: 342 - protein, 15 aa, Homo sapiens
  MOD_RES (10)..(10): Phosphorylated Tyrosine
LHCIVTYCEYCQEEK

SEQ ID NO: 343 - protein, 12 aa, Homo sapiens
  MOD_RES (6)..(6): Phosphorylated Tyrosine
CVTCNYCSQLCK

SEQ ID NO: 344 - protein, 8 aa, Homo sapiens
  MOD_RES (6)..(6): Phosphorylated Tyrosine
KFQDWYYK

SEQ ID NO: 345 - protein, 15 aa, Homo sapiens
  MOD_RES (7)..(7): Phosphorylated Tyrosine
GPENLHYDQGCQTSR

SEQ ID NO: 346 - protein, 11 aa, Homo sapiens
  MOD_RES (3)..(3): Phosphorylated Tyrosine
ATYCKPHMQTK

SEQ ID NO: 347 - protein, 18 aa, Homo sapiens
  MOD_RES (11)..(11): Phosphorylated Tyrosine
HFNIHTGEKPYECLQCGK

SEQ ID NO: 348 - protein, 16 aa, Homo sapiens
  MOD_RES (3)..(3): Phosphorylated Tyrosine
ENYTQNTIQVAIQSLR

SEQ ID NO: 349 - protein, 10 aa, Homo sapiens
  MOD_RES (4)..(4): Phosphorylated Tyrosine
YQNYIMEGVK

SEQ ID NO: 350 - protein, 18 aa, Homo sapiens
  MOD_RES (17)..(17): Phosphorylated Tyrosine
SVVCPICASMPWGDPNYR

SEQ ID NO: 351 - protein, 12 aa, Homo sapiens
  MOD_RES (3)..(3): Phosphorylated Tyrosine
SGYGFNEPEQSR

SEQ ID NO: 352 - protein, 33 aa, Homo sapiens
  MOD_RES (7)..(7): Phosphorylated Tyrosine
NLSSDKYGDTSYHDEEEDEYEAEDDEEEEDEGR

SEQ ID NO: 353 - protein, 27 aa, Homo sapiens
  MOD_RES (6)..(6): Phosphorylated Tyrosine
YGDTSYHDEEEDEYEAEDDEEEEDEGR

SEQ ID NO: 354 - protein, 27 aa, Homo sapiens
  MOD_RES (14)..(14): Phosphorylated Tyrosine
YGDTSYHDEEEDEYEAEDDEEEEDEGR

SEQ ID NO: 355 - protein, 14 aa, Homo sapiens
  MOD_RES (6)..(6): Phosphorylated Tyrosine
TYASGYAHYEEQEN

SEQ ID NO: 356 - protein, 19 aa, Homo sapiens
  MOD_RES (17)..(17): Phosphorylated Tyrosine
GPPPVQSSLNMHSVPSYLT

SEQ ID NO: 357 - protein, 14 aa, Homo sapiens
  MOD_RES (11)..(11): Phosphorylated Tyrosine
PFQCPECDKSYCIR

SEQ ID NO: 358 - protein, 13 aa, Homo sapiens
  MOD_RES (2)..(2): Phosphorylated Tyrosine
AYEYQEYGQKPYK

SEQ ID NO: 359 - protein, 13 aa, Homo sapiens
  MOD_RES (4)..(4): Phosphorylated Tyrosine
AYEYQEYGQKPYK

SEQ ID NO: 360 - protein, 13 aa, Homo sapiens
  MOD_RES (7)..(7): Phosphorylated Tyrosine
AYEYQEYGQKPYK

SEQ ID NO: 361 - protein, 13 aa, Homo sapiens
  MOD_RES (12)..(12): Phosphorylated Tyrosine
AYEYQEYGQKPYK

SEQ ID NO: 362 - protein, 19 aa, Homo sapiens
  MOD_RES (12)..(12): Phosphorylated Tyrosine
IQCPNQGCEAVYSSVSGLK

SEQ ID NO: 363 - protein, 13 aa, Homo sapiens
  MOD_RES (3)..(3): Phosphorylated Tyrosine
SPYTCPECGAICR

SEQ ID NO: 364 - protein, 13 aa, Homo sapiens
  MOD_RES (9)..(9): Phosphorylated Tyrosine
QCSKAFPFYSSYR

SEQ ID NO: 365 - protein, 13 aa, Homo sapiens
  MOD_RES (12)..(12): Phosphorylated Tyrosine
QCSKAFPFYSSYR

SEQ ID NO: 366 - protein, 15 aa, Homo sapiens
  MOD_RES (8)..(8): Phosphorylated Tyrosine
VHTGEKPYICKECGK

SEQ ID NO: 367 - protein, 10 aa, Homo sapiens
  MOD_RES (7)..(7): Phosphorylated Tyrosine
YHHTGEYPYK

SEQ ID NO: 368 - protein, 29 aa, Homo sapiens
  MOD_RES (3)..(3): Phosphorylated Tyrosine
VLYKEVMLENYINLVSIGYRGTKPDSLFK

SEQ ID NO: 369 - protein, 13 aa, Homo sapiens
  MOD_RES (3)..(3): Phosphorylated Tyrosine
SPYCCPECGVLCR

SEQ ID NO: 370 - protein, 23 aa, Homo sapiens
  MOD_RES (7)..(7): Phosphorylated Tyrosine
AEADKIYSFTDNAPSPSIGGSSR

SEQ ID NO: 371 - protein, 16 aa, Homo sapiens
  MOD_RES (11)..(11): Phosphorylated Tyrosine
DHSFFIPDIEYLSDIK

SEQ ID NO: 372 - protein, 20 aa, Homo sapiens
  MOD_RES (1)..(1): Phosphorylated Tyrosine
YLFTALLPHDPDLAYRLALR

SEQ ID NO: 373 - protein, 9 aa, Homo sapiens
  MOD_RES (1)..(1): Phosphorylated Tyrosine
YQASHPNLR

SEQ ID NO: 374 - protein, 12 aa, Homo sapiens
  MOD_RES (8)..(8): Phosphorylated Tyrosine
IGDGMNAYVAYK

SEQ ID NO: 375 - protein, 14 aa, Homo sapiens
  MOD_RES (6)..(6): Phosphorylated Tyrosine
YLHVGYIVPPAPEK

SEQ ID NO: 376 - protein, 11 aa, Homo sapiens
  MOD_RES (7)..(7): Phosphorylated Tyrosine
FVVYNVYMAGR

SEQ ID NO: 377 - protein, 14 aa, Homo sapiens
  MOD_RES (10)..(10): Phosphorylated Tyrosine
WVDGVDEIQYDVGR

SEQ ID NO: 378 - protein, 28 aa, Homo sapiens
  MOD_RES (16)..(16): Phosphorylated Tyrosine
NKDDRPNTDIVDEDPYAIQIISWCPESR

SEQ ID NO: 379 - protein, 19 aa, Homo sapiens
  MOD_RES (17)..(17): Phosphorylated Tyrosine
FEEEDDDDDFPAPSDGYYR

SEQ ID NO: 380 - protein, 19 aa, Homo sapiens
  MOD_RES (18)..(18): Phosphorylated Tyrosine
FEEEDDDDDFPAPSDGYYR

SEQ ID NO: 381 - protein, 13 aa, Homo sapiens
  MOD_RES (9)..(9): Phosphorylated Tyrosine
GDFDENLNYPEQK

SEQ ID NO: 382 - protein, 15 aa, Homo sapiens
  MOD_RES (8)..(8): Phosphorylated Tyrosine
SEDPDQQYLILNTAR

SEQ ID NO: 383 - protein, 23 aa, Homo sapiens
  MOD_RES (17)..(17): Phosphorylated Tyrosine
AGNYEEALQLYQHAVQYFLHVVK

(19) United States
(12) Patent Application Publication
(10) Pub. No.: US 2010/0159477 A1
(43) Pub. Date: Jun. 24, 2010

(54) Reagents for the detection of protein phosphorylation in signaling pathways

(76) Inventors: Peter Hornbeck, Magnolia, MA (US); Valerie Goss, Seabrook, NH (US); Kimberly Lee, Seattle, WA (US); Ting-Lei Gu, Woburn, MA (US); Albrecht Moritz, Salem, MA (US)

Correspondence Address: Nancy Chiu Wilker, Ph.D.;Chief Intellectual Property Counsel, CELL SIGNALING TECHNOLOGY, INC., 3 Trask Lane, Danvers, MA, 01923, US

(21) Appl. No.: 12/309,311

(22) PCT Filed: Jul. 13, 2007
(86) PCT No.: PCT/US07/73537

§ 371 Date: Feb. 4, 2010

Related U.S. Application Data
(60) Provisional application No. 60/830,724, filed on Jul. 13, 2006.

(51) Int. Cl. G01N 33/573; G01N 33/53; C07K 16/00; C07K 7/06; C07K 7/08; C07K 14/00; C12N 5/071; C12N 5/16; C12N 5/18

(52) U.S. Cl. 435/7.4; 435/7.1; 530/387.1; 530/328; 530/327; 530/326; 530/325; 530/324; 435/326

(57) ABSTRACT

The invention discloses novel phosphorylation sites identified in signal transduction proteins and pathways, and provides phosphorylation-site specific antibodies and heavy-isotope labeled peptides (AQUA peptides) for the selective detection and quantification of these phosphorylated sites/proteins, as well as methods of using the reagents for such purpose. Among the phosphorylation sites identified are sites occurring in the following protein types: adaptor/scaffold proteins, adhesion/extracellular matrix protein, apoptosis proteins, calcium binding proteins, cell cycle regulation proteins, chaperone proteins, chromatin, DNA binding/repair/replication proteins, cytoskeletal proteins, endoplasmic reticulum or golgi proteins, enzyme proteins, G/regulator proteins, inhibitor proteins, motor/contractile proteins, phosphatase, protease, Ser/Thr protein kinases, Protein kinase (Tyr)s, receptor/channel/cell suface proteins, RNA binding proteins, transcriptional regulators, tumor suppressor proteins, ubiquitan conjugating system proteins and proteins of unknown function.

### RELATED APPLICATIONS

Pursuant to 35 U.S.C. §119(e) this application claims the benefit of, and priority to, provisional application U.S. Ser. No. 60/830,724, filed Jul. 13, 2006, the disclosure of which is incorporated herein, in its entirety, by reference.

### TECHNICAL FIELD

The invention relates generally to a variety of moieties and tools for the detection of protein phosphorylation. Moreover, the invention relates to the use of the same for diagnostic and therapeutic purposes.

### BACKGROUND

The activation of proteins by post-translational modification is an important cellular mechanism for regulating most aspects of biological organization and control, including growth, development, homeostasis, and cellular communication. Cellular signal transduction pathways involve protein kinases, protein phosphatases, and phosphoprotein-interacting domain (e.g., SH2, PTB, WW, FHA, 14-3-3) containing cellular proteins to provide multidimensional, dynamic and reversible regulation of many biological activities. See e.g., Sawyer et al., Med. Chem. 1(3): 293-319 (2005).

Protein phosphorylation on a proteome-wide scale is extremely complex as a result of three factors: the large number of modifying proteins, e.g. kinases, encoded in the genome, the much larger number of sites on substrate proteins that are modified by these enzymes, and the dynamic nature of protein expression during growth, development, disease states, and aging. The human genome, for example, encodes over 520 different protein kinases, making them the most abundant class of enzymes known. See Hunter, Nature 411: 355-65 (2001). Most kinases phosphorylate many different substrate proteins, at distinct tyrosine, serine, and/or threonine residues. Indeed, it is estimated that one-third of all proteins encoded by the human genome are phosphorylated, and many are phosphorylated at multiple sites by different kinases. See Graves et al., Pharmacol. Ther. 82: 111-21 (1999).

Many of these phosphorylation sites regulate critical biological processes and may prove to be important for diagnostic or therapeutic modalities useful in the treatment and management of many pathological conditions and diseases, including inter alia cancer, developmental disorders, as as inflammatory, immune, metabolic and bone diseases.

For example, of the more than 100 dominant oncogenes identified to date, 46 are protein kinases. See Hunter, supra. Understanding which proteins are modified by these kinases will greatly expand our understanding of the molecular mechanisms underlying oncogenic transformation. Therefore, the identification of, and ability to detect, phosphorylation sites on a wide variety of cellular proteins is crucially important to understanding the key signaling proteins and pathways implicated in the progression of many disease states.

Understanding reversible protein phosphorylation and its role in the operation and interrelationship between cellular components and functions provides the opportunity to gain a finer appreciation of cellular regulation. In spite of the importance of protein modification, phosphorylation is not yet well understood due to the extraordinary complexity of signaling pathways, and the slow development of the technology necessary to unravel it.

In many instances, such knowledge is likely to provide valuable tools useful to evaluate, and possibly to manipulate target pathways, ultimately altering the functional status of a given cell for a variety of purposes.

The importance of protein kinase-regulated signal transduction pathways is underscored by a number of drugs designed to treat various cancer types by the inhibition of target protein kinases at the apex or intermediary levels of pathways implicated in cancer development. See Stern et al., Expert Opin. Ther. Targets 9(4):851-60 (2005).

Leukemia, a disease in which a number of underlying signal transduction events have been elucidated, has become a disease model for phosphoproteomic research and development efforts. As such, it represent a paradigm leading the way for many other programs seeking to address many classes of diseases (See, Harrison's Principles of Internal Medicine, McGraw-Hill, New York, N.Y.)

Depending on the cell type involved and the rate by which the disease progresses leukemia can be defined as acute or chronic myelogenous leukemia (AML or CML), or acute and chronic lymphocytic leukemia (ALL or CLL).

Most varieties of leukemia are generally characterized by genetic alterations e.g., chromosomal translocations, deletions or point mutations resulting in the constitutive activation of protein kinase genes, and their products, particularly tyrosine kinases. The most well known alteration is the oncogenic role of the chimeric BCR-Abl gene. See Nowell, Science 132: 1497 (1960)). The resulting BCR-Abl kinase protein is constitutively active and elicits characteristic signaling pathways that have been shown to drive the proliferation and survival of CML cells (see Daley, Science 247: 824-830 (1990); Raitano et al., Biochim. Biophys. Acta. December 9; 1333(3): F201-16 (1997)).

The recent success of Imanitib (also known as STI571 or Gleevec®), the first molecularly targeted compound designed to specifically inhibit the tyrosine kinase activity of BCR-Abl, provided critical confirmation of the central role of BCR-Abl signaling in the progression of CML (see Schindler et al., Science 289: 1938-1942 (2000); Nardi et al., Curr. Opin. Hematol. 11: 35-43 (2003)).

The success of Gleevec® now serves as a paradigm for the development of targeted drugs designed to block the activity of other tyrosine kinases known to be involved in many diseased including leukemias and other malignancies (see, e.g., Sawyers, Curr. Opin. Genet. Dev. February; 12(1): 111-5 (2002); Druker, Adv. Cancer Res. 91:1-30 (2004)). For example, recent studies have demonstrated that mutations in the FLT3 gene occur in one third of adult patients with AML. FLT3 (Fms-like tyrosine kinase 3) is a member of the class III receptor tyrosine kinase (RTK) family including FMS, platelet-derived growth factor receptor (PDGFR) and c-KIT (see Rosnet et al., Crit. Rev. Oncog. 4: 595-613 (1993). In 20-27% of patients with AML, an internal tandem duplication in the juxta-membrane region of FLT3 can be detected (see Yokota et al., Leukemia 11: 1605-1609 (1997)). Another 7% of patients have mutations within the active loop of the second kinase domain, predominantly substitutions of aspartate residue 835 (D835), while additional mutations have been described (see Yamamoto et al., Blood 97: 2434-2439 (2001); Abu-Duhier et al., Br. J. Haematol. 113: 983-988 (2001)). Expression of mutated FLT3 receptors results in constitutive tyrosine phosphorylation of FLT3, and subsequent phosphorylation and activation of downstream molecules such as STAT5, Akt and MAPK, resulting in factor-independent growth of hematopoietic cell lines.

Altogether, FLT3 is the single most common activated gene in AML known to date. This evidence has triggered an intensive search for FLT3 inhibitors for clinical use leading to at least four compounds in advanced stages of clinical development, including: PKC412 (by Novartis), CEP-701 (by Cephalon), MLN518 (by Millenium Pharmaceuticals), and SU5614 (by Sugen/Pfizer) (see Stone et al., Blood (in press)(2004); Smith et al., Blood 103: 3669-3676 (2004); Clark et al., Blood 104: 2867-2872 (2004); and Spiekerman et al., Blood 101: 1494-1504 (2003)).

There is also evidence indicating that kinases such as FLT3, c-KIT and Abl are implicated in some cases of ALL (see Cools et al., Cancer Res. 64: 6385-6389 (2004); Hu, Nat. Genet. 36: 453-461 (2004); and Graux et al., Nat. Genet. 36: 1084-1089 (2004)). In contrast, very little is know regarding any causative role of protein kinases in CLL, except for a high correlation between high expression of the tyrosine kinase ZAP70 and the more aggressive form of the disease (see Rassenti et al., N. Eng. J. Med. 351: 893-901 (2004)).

Despite the identification of a few key molecules involved in progression of leukemia, the vast majority of signaling protein changes underlying this disease remains unknown. There is, therefore, relatively scarce information about kinase-driven signaling pathways and phosphorylation sites relevant to the different types of leukemia. This has hampered a complete and accurate understanding of how protein activation within signaling pathways is driving these complex cancers. Accordingly, there is a continuing and pressing need to unravel the molecular mechanisms of kinase-driven oncogenesis in leukemia by identifying the downstream signaling proteins mediating cellular transformation in this disease. Identifying particular phosphorylation sites on such signaling proteins and providing new reagents, such as phospho-specific antibodies and AQUA peptides, to detect and quantify them remains particularly important to advancing our understanding of the biology of this disease.

Presently, diagnosis of leukemia is made by tissue biopsy and detection of different cell surface markers. However, misdiagnosis can occur since some leukemia cases can be negative for certain markers, and because these markers may not indicate which genes or protein kinases may be deregulated. Although the genetic translocations and/or mutations characteristic of a particular form of leukemia can be sometimes detected, it is clear that other downstream effectors of constitutively active kinases having potential diagnostic, predictive, or therapeutic value, remain to be elucidated. Accordingly, identification of downstream signaling molecules and phosphorylation sites involved in different types of leukemia and development of new reagents to detect and quantify these sites and proteins may lead to improved diagnostic/prognostic markers, as well as novel drug targets, for the detection and treatment of this disease.

### SUMMARY OF THE INVENTION

Several novel protein phosphorylation sites have been identified in a variety of cell lines. Such novel phosphorylation sites (tyrosine), and their corresponding parent proteins are reported (see Table 1). The elucidation of these sites at long last provides the elements necessary to attain those much needed proteomics tools and modalities.

The invention discloses novel phosphorylation sites identified in signal transduction proteins and pathways underlying various disease states including for example human leukemias. The invention thus provides new reagents, including phosphorylation-site specific antibodies and AQUA peptides, for the selective detection and quantification of these phosphorylated sites/proteins. Also provided are methods of using the reagents of the invention for the detection and quantification of the disclosed phosphorylation sites.

### BRIEF DESCRIPTION OF THE DRAWINGS

FIG. 1—Is a diagram broadly depicting the immunoaffinity isolation and mass-spectrometric characterization methodology (IAP) employed to identify the novel phosphorylation sites disclosed herein.

FIG. 2—Is a table (corresponding to Table 1) enumerating the Leukemia signaling protein phosphorylation sites disclosed herein: Column A=the name of the parent protein; Column B=the SwissProt accession number for the protein (human sequence); Column C=the protein type/classification; Column D=the tyrosine residue (in the parent protein amino acid sequence) at which phosphorylation occurs within the phosphorylation site; Column E=the phosphorylation site sequence encompassing the phosphorylatable residue (residue at which phosphorylation occurs (and corresponding to the respective entry in Column D) appears in lowercase; Column F=the type of leukemia in which the phosphorylation site was discovered; and Column G=the cell type(s), tissue(s) and/or patient(s) in which the phosphorylation site was discovered.

FIG. 3—is an exemplary mass spectrograph depicting the detection of the tyrosine 786 phosphorylation site in TrkC (see Row 139 in FIG. 2/Table 1), as further described in Example 1 (red and blue indicate ions detected in MS/MS spectrum); Y* indicates the phosphorylated tyrosine (shown as lowercase “y” in FIG. 2).

FIG. 4—is an exemplary mass spectrograph depicting the detection of the tyrosine 192 phosphorylation site in HSP90B (see Row 30 in FIG. 2/Table 1), as further described in Example 1 (red and blue indicate ions detected in MS/MS spectrum); Y* indicates the phosphorylated tyrosine (shown as lowercase “y” in FIG. 2).

FIG. 5—is an exemplary mass spectrograph depicting the detection of the tyrosine 328 phosphorylation site in TOP2A (see Row 87 in FIG. 2/Table 1), as further described in Example 1 (red and blue indicate ions detected in MS/MS spectrum); Y* indicates the phosphorylated serine (shown as lowercase “y” in FIG. 2).

FIG. 6—is an exemplary mass spectrograph depicting the detection of the tyrosine 15 phosphorylation site in SNRPN (see Row 157 in FIG. 2/Table 1), as further described in Example 1 (red and blue indicate ions detected in MS/MS spectrum); Y* indicates the phosphorylated tyrosine (shown as lowercase “y” in FIG. 2)

FIG. 7—is an exemplary mass spectrograph depicting the detection of the tyrosine 507 phosphorylation site in VPS35 (see Row 383 in FIG. 2/Table 1), as further described in Example 1 (red and blue indicate ions detected in MS/MS spectrum); Y* indicates the phosphorylated tyrosine (shown as lowercase “y” in FIG. 2).

FIG. 8—is an exemplary mass spectrograph depicting the detection of the tyrosine 192 phosphorylation site in TAGLN3 (see Row 66 in FIG. 2/Table 1), as further described in Example 1 (red and blue indicate ions detected in MS/MS spectrum); Y* indicates the phosphorylated tyrosine (shown as lowercase “y” in FIG. 2).

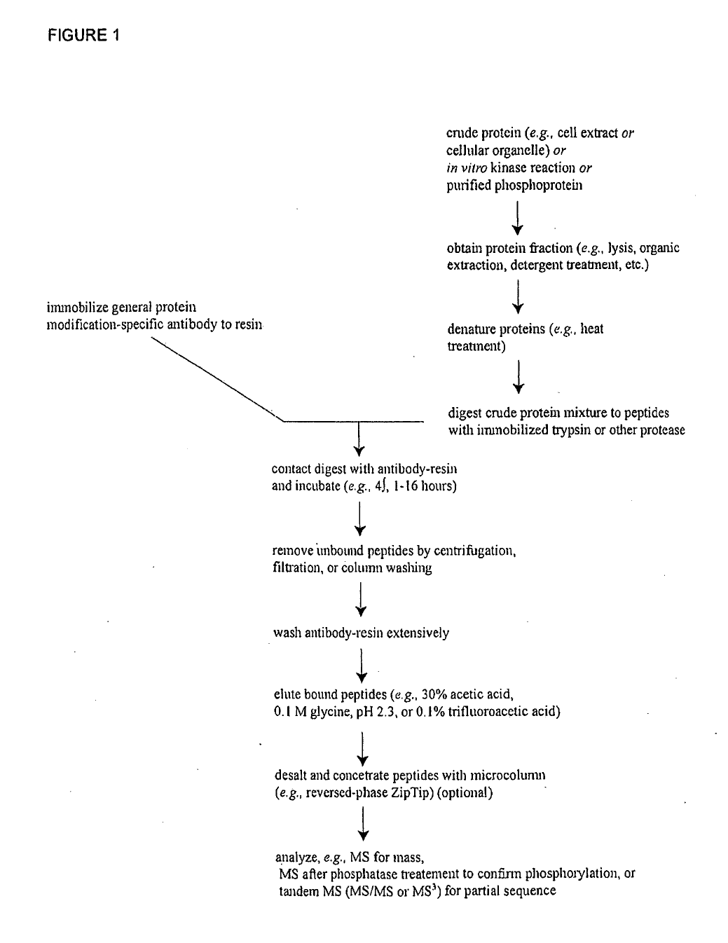

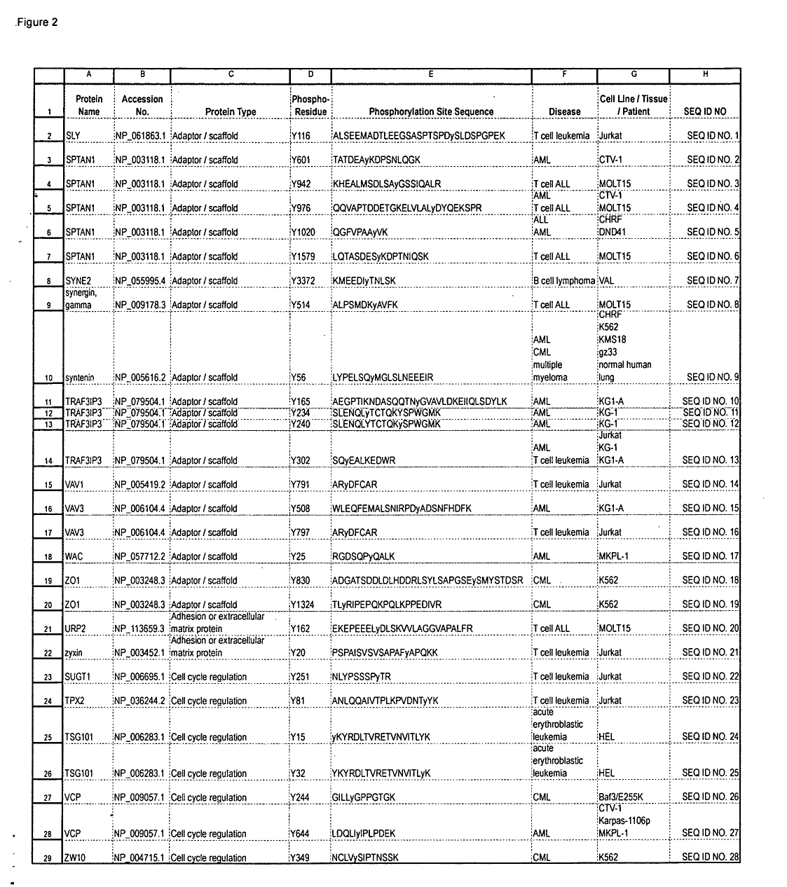

### DETAILED DESCRIPTION

Several novel protein phosphorylation sites have been identified in a variety of cell lines. Such novel phosphorylation sites (tyrosine), and their corresponding parent proteins are reported (see Table 1). The elucidation of these sites at long last provides the elements necessary to attain those much needed proteomics tools and modalities.

The disclosure of the phosphorylation sites provides the key to the production of new moieties, compositions and methods to specifically detect and/or to quantify these phosphorylated sites/proteins. Such moieties include for example reagents, such as phosphorylation site-specific antibodies and AQUA peptides (heavy-isotope labeled peptides). Such reagents are highly useful, inter alia, for studying signal transduction events underlying the progression of many diseases known or suspected to involve protein phosphorylation e.g., leukemia in a mammal. Accordingly, the invention provides novel reagents—phospho-specific antibodies and AQUA peptides—for the specific detection and/or quantification of a target signaling protein/polypeptide (e.g., a signaling protein/polypeptide implicated in leukemia) only when phosphorylated (or only when not phosphorylated) at a particular phosphorylation site disclosed herein. The invention also provides methods of detecting and/or quantifying one or more phosphorylated target signaling protein/polypeptide using the phosphorylation-site specific antibodies and AQUA peptides of the invention.

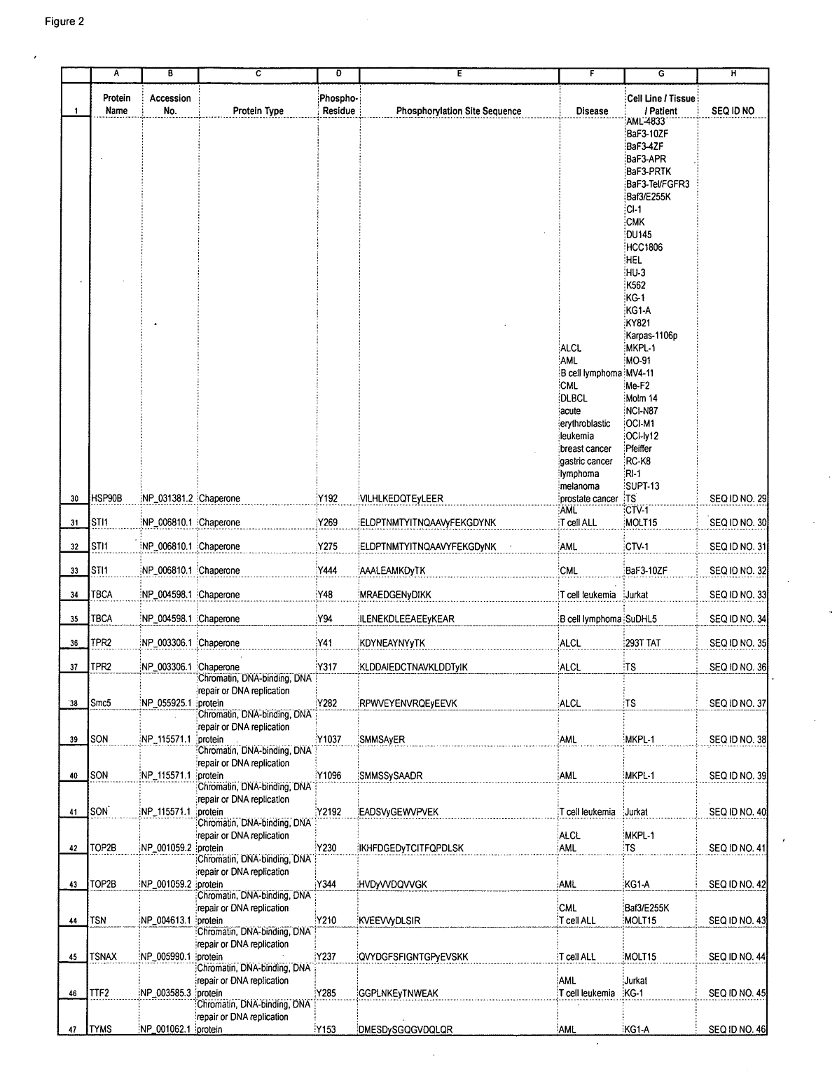

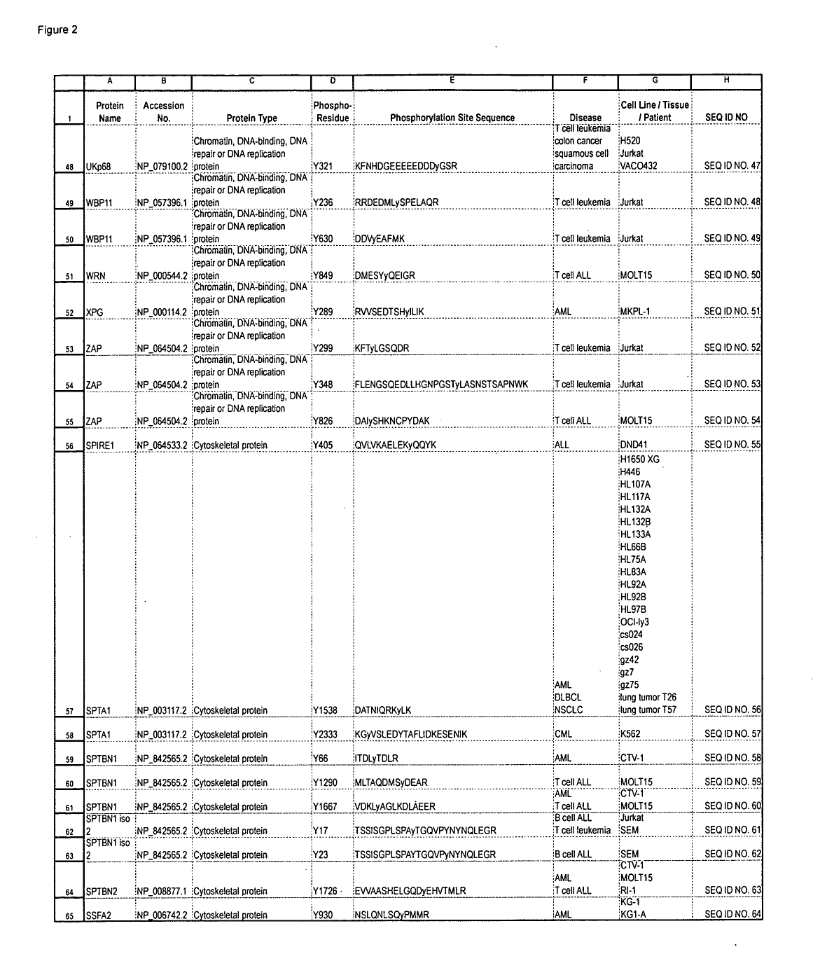

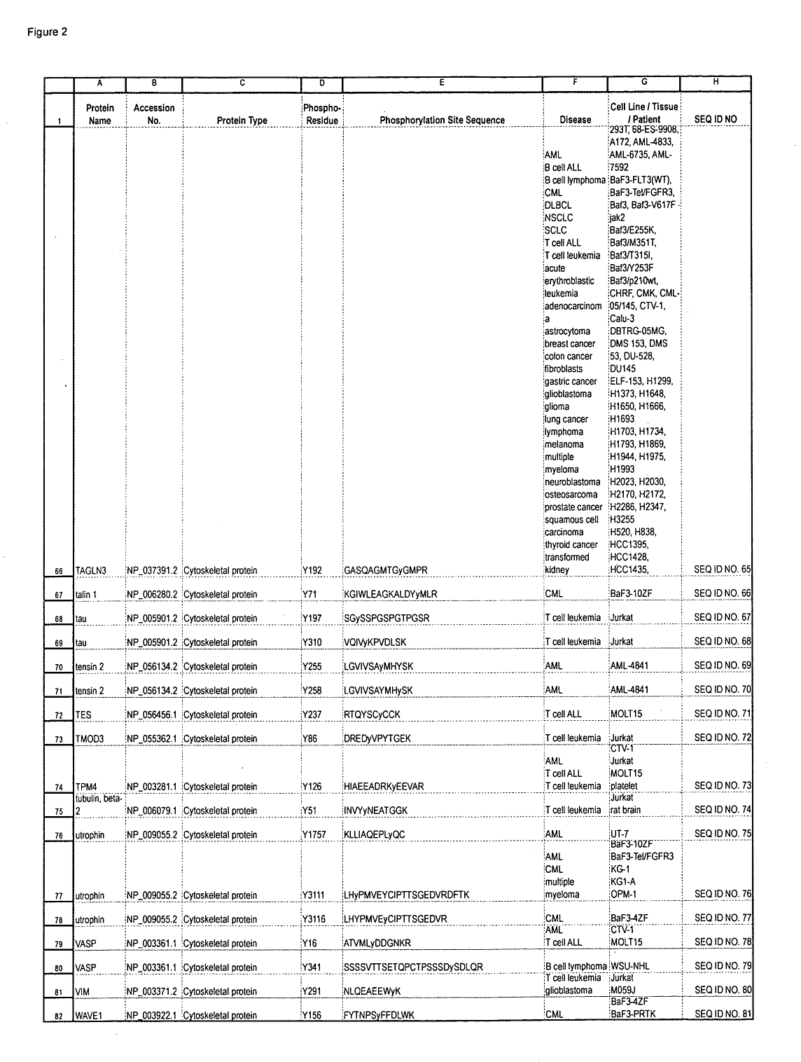

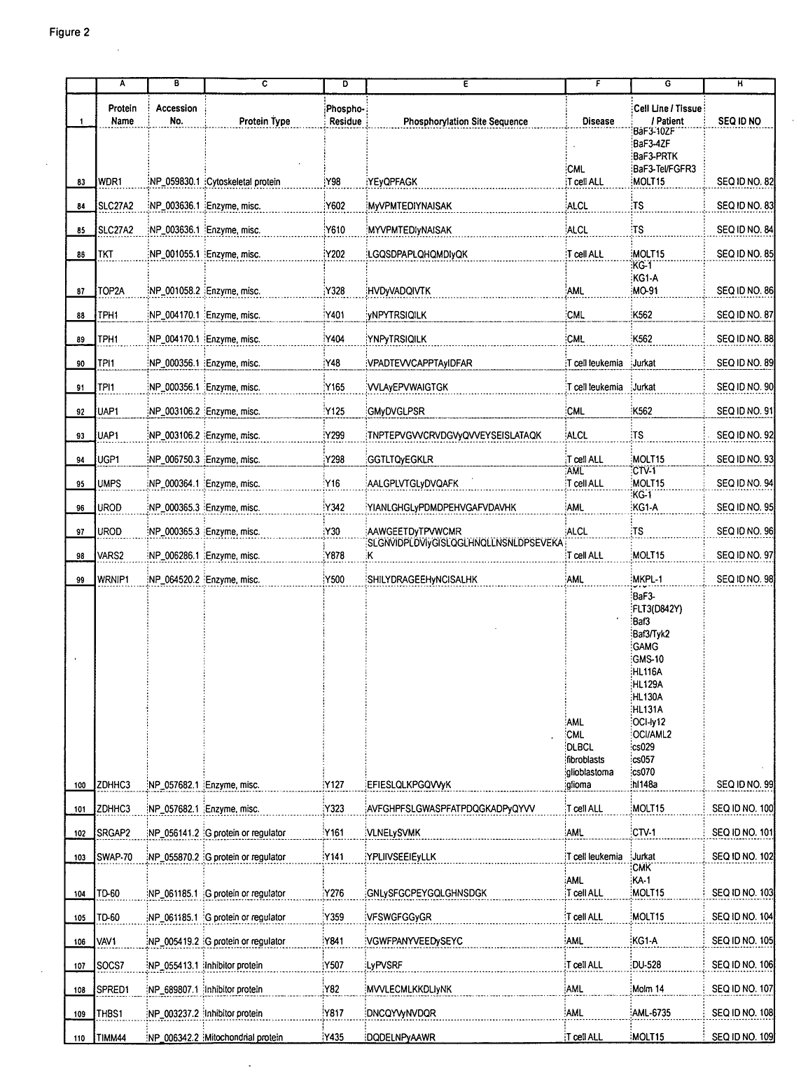

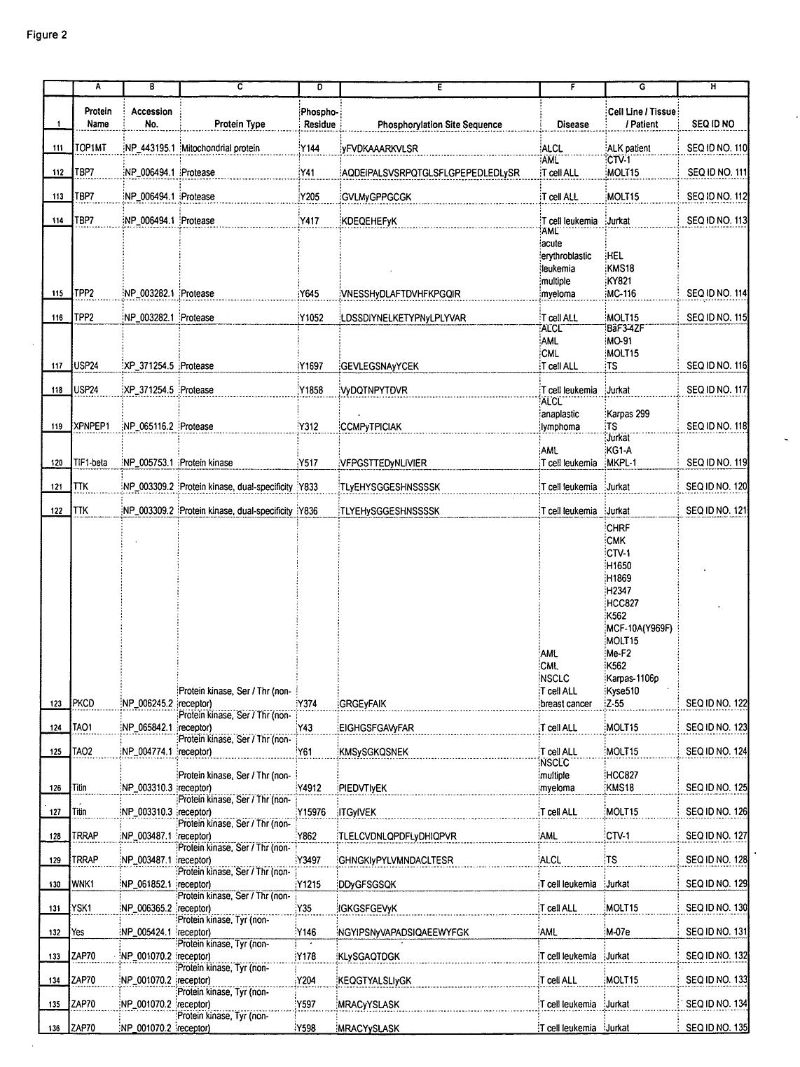

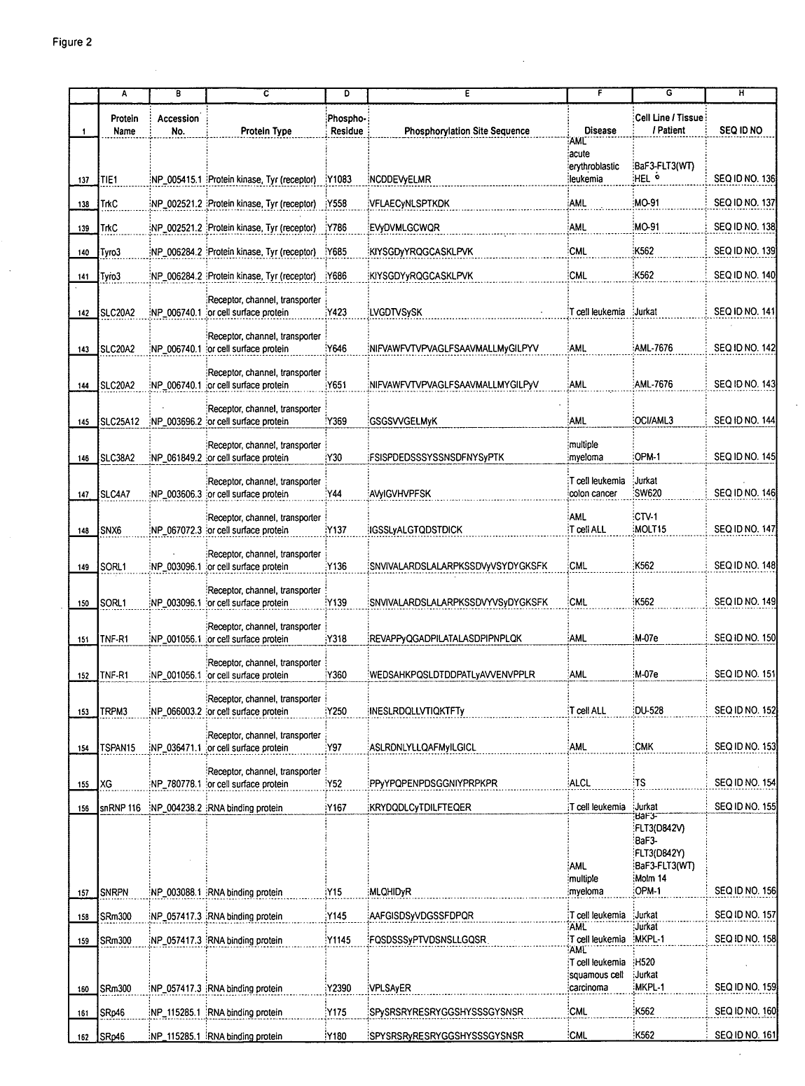

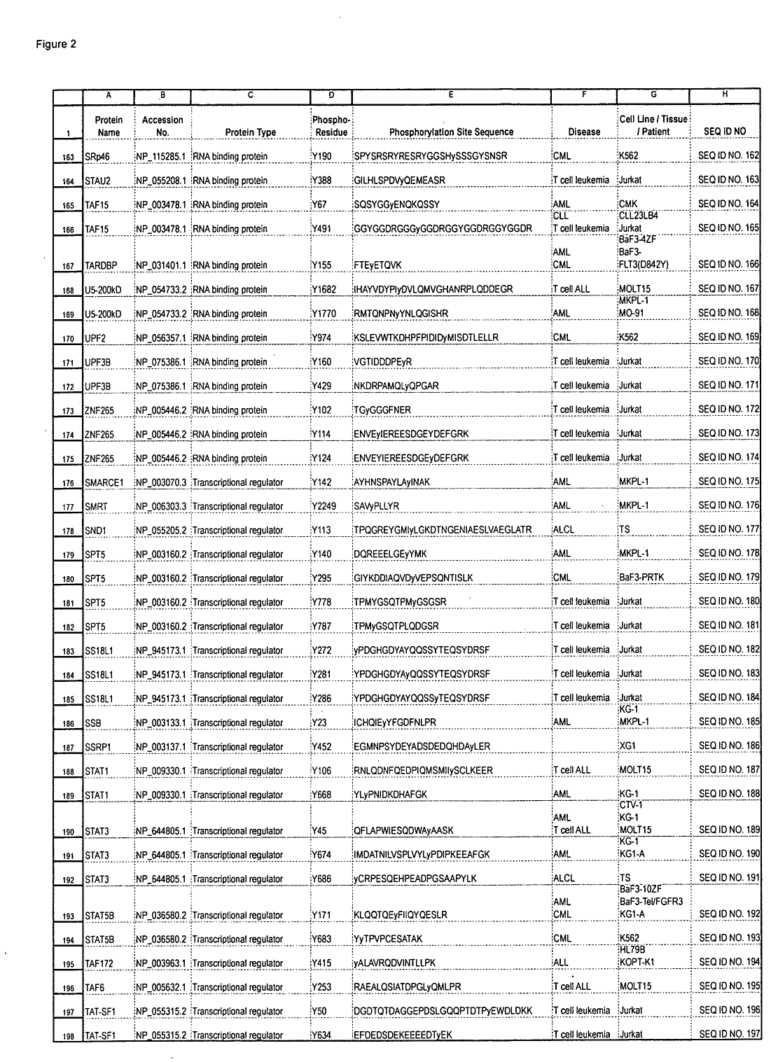

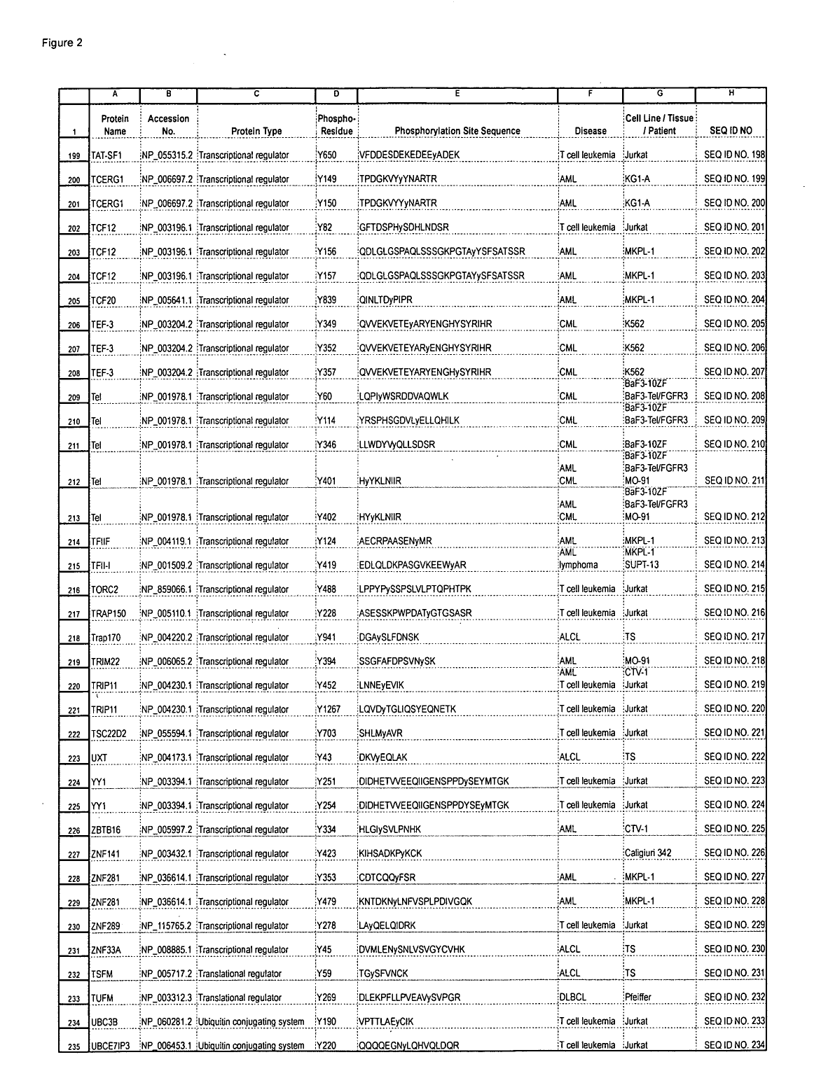

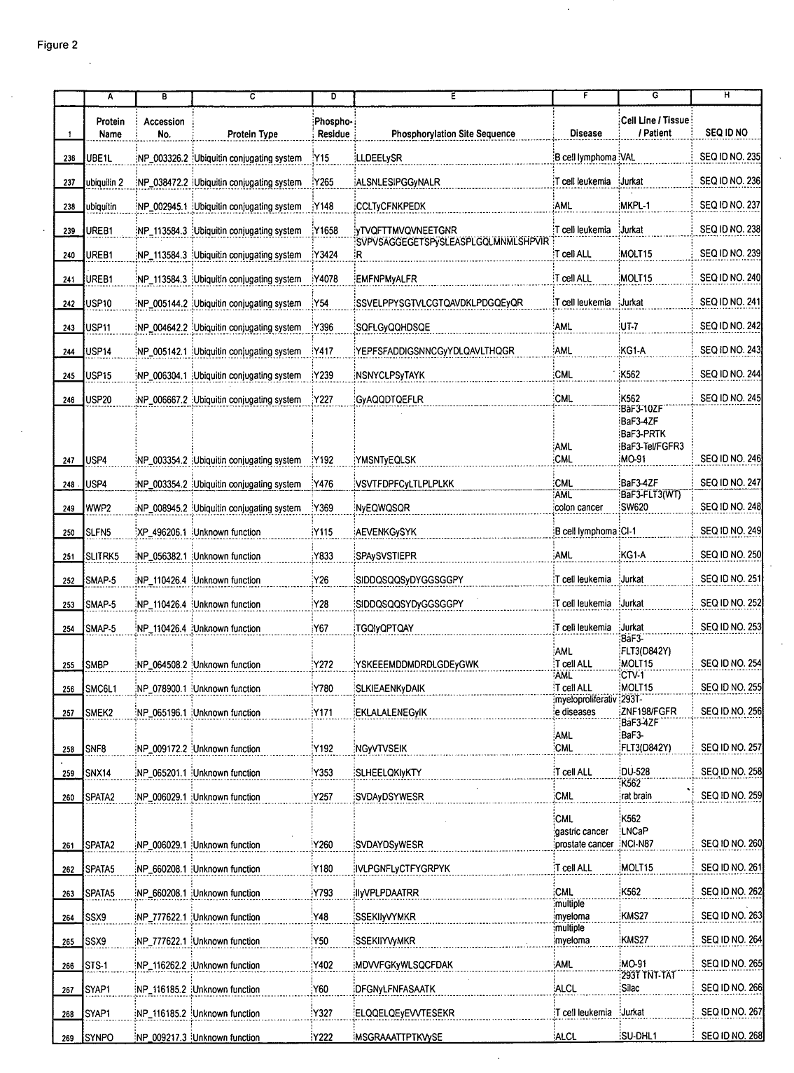

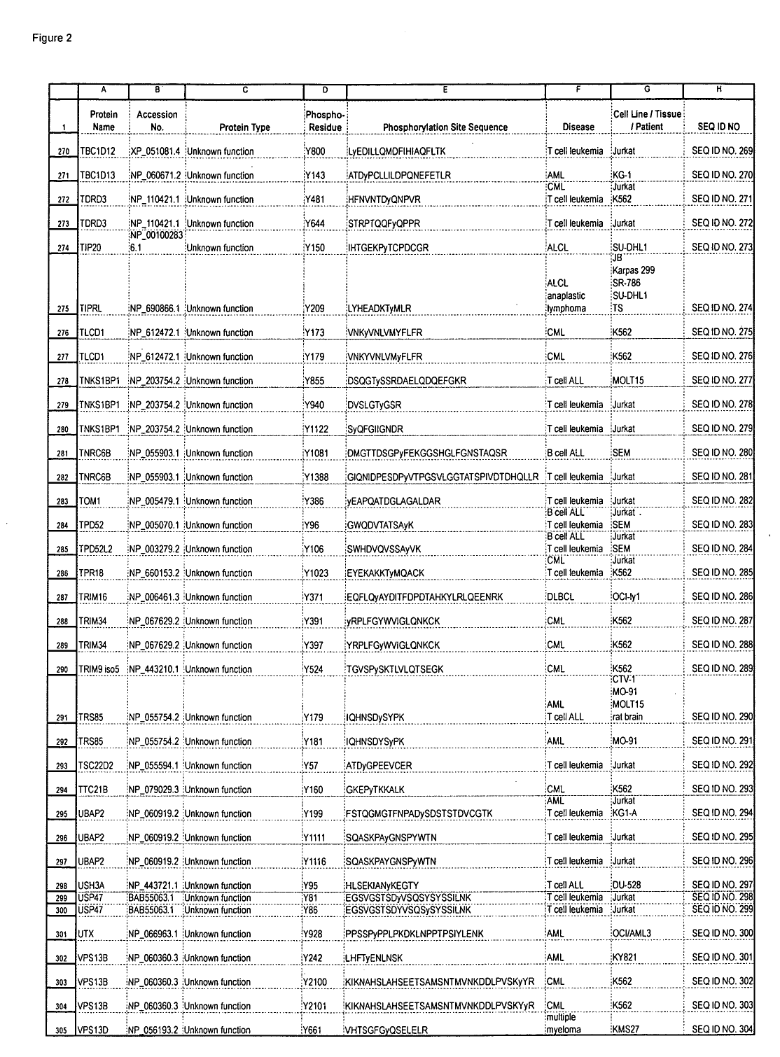

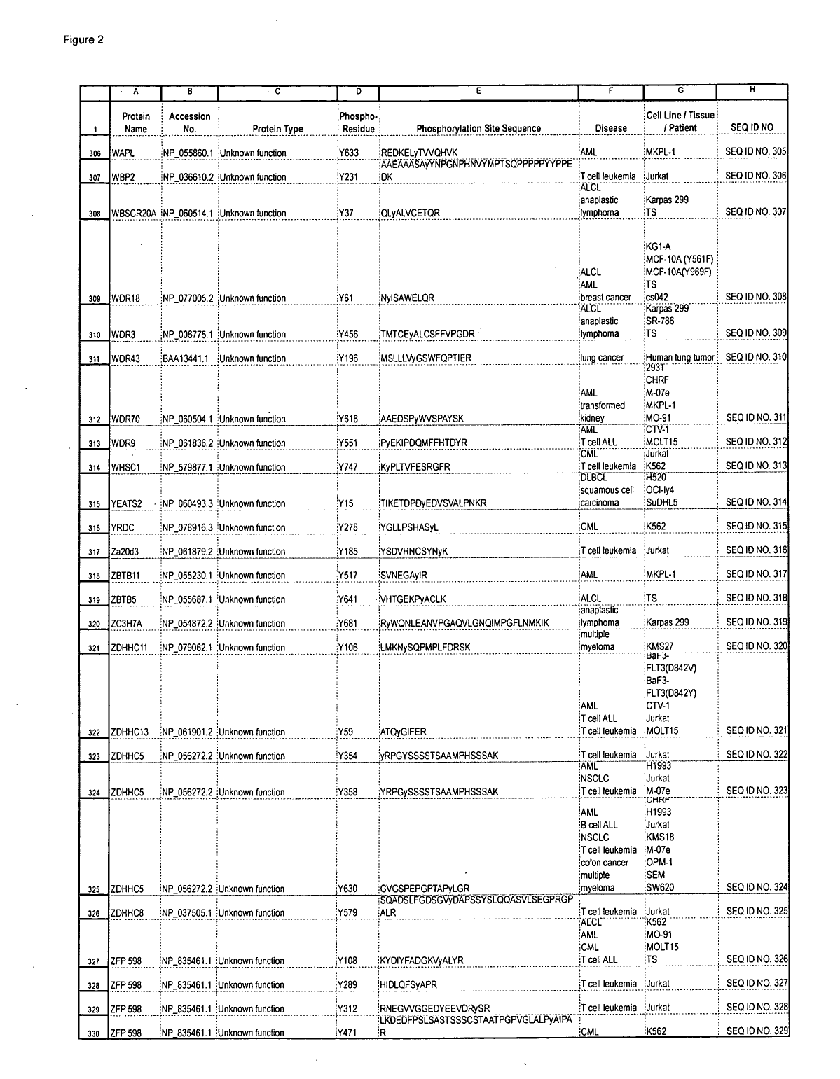

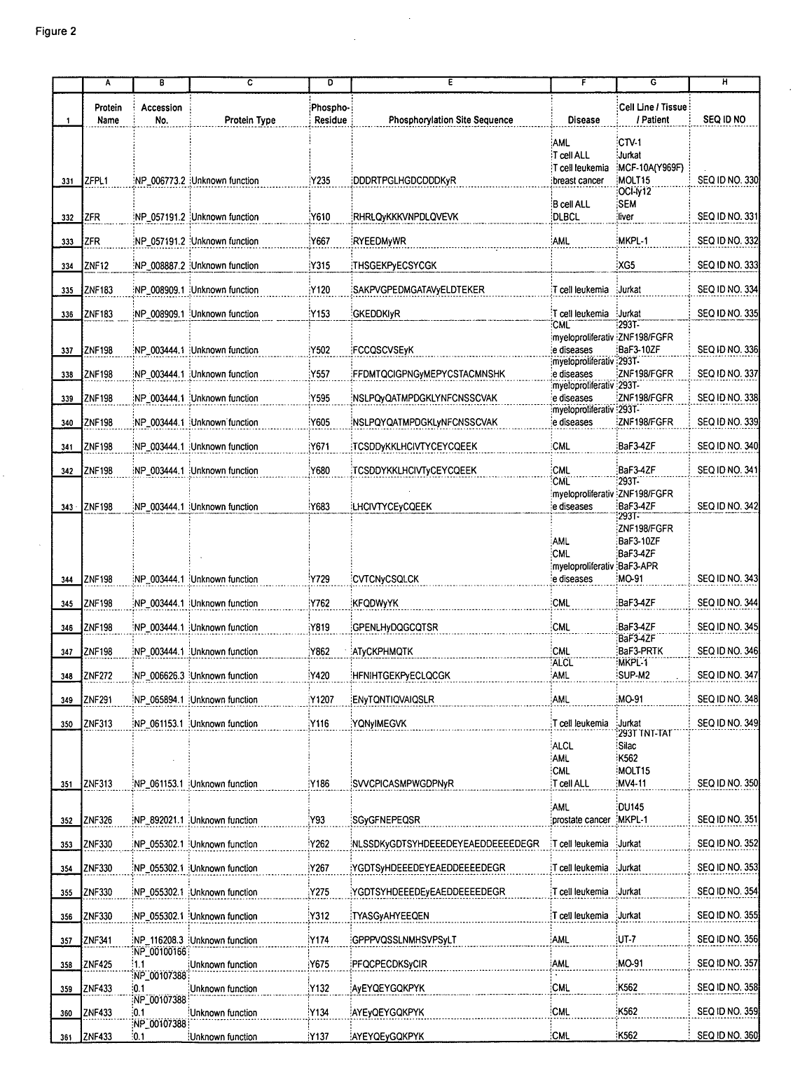

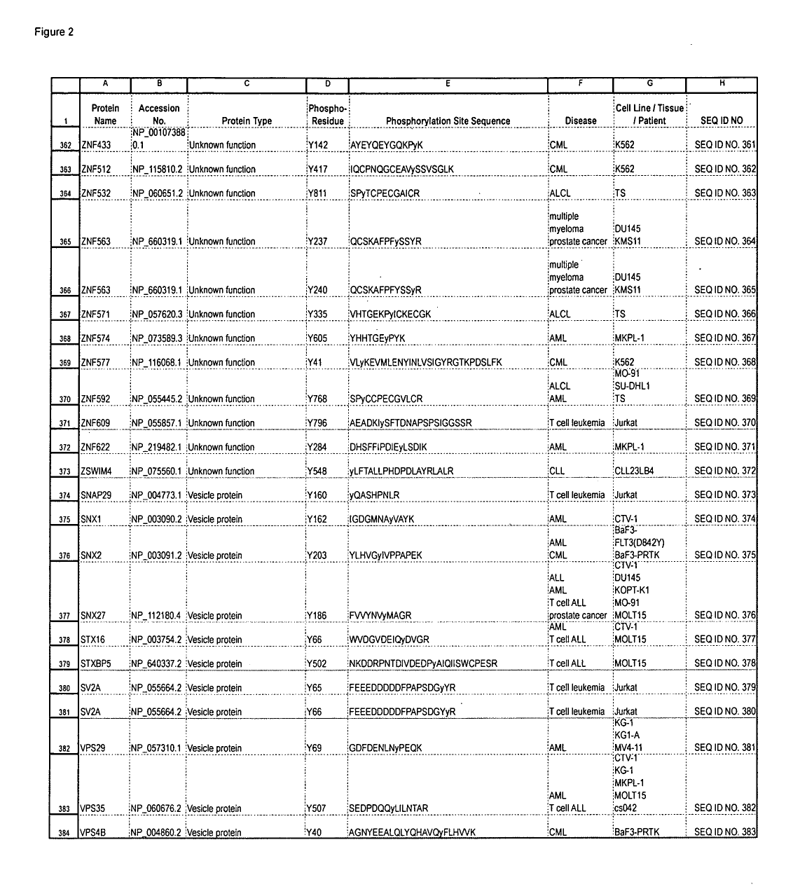

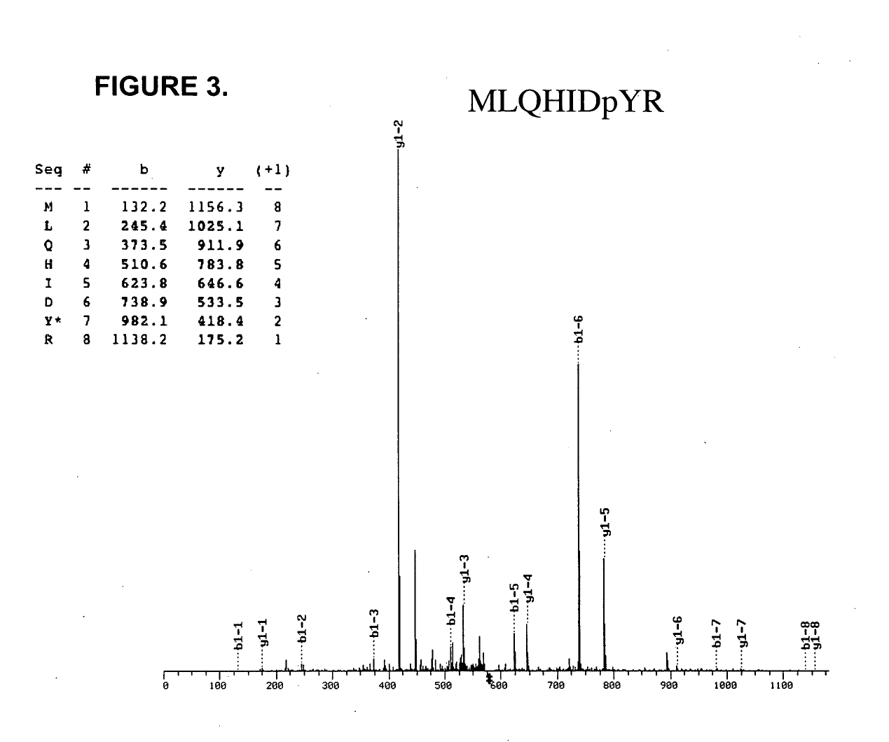

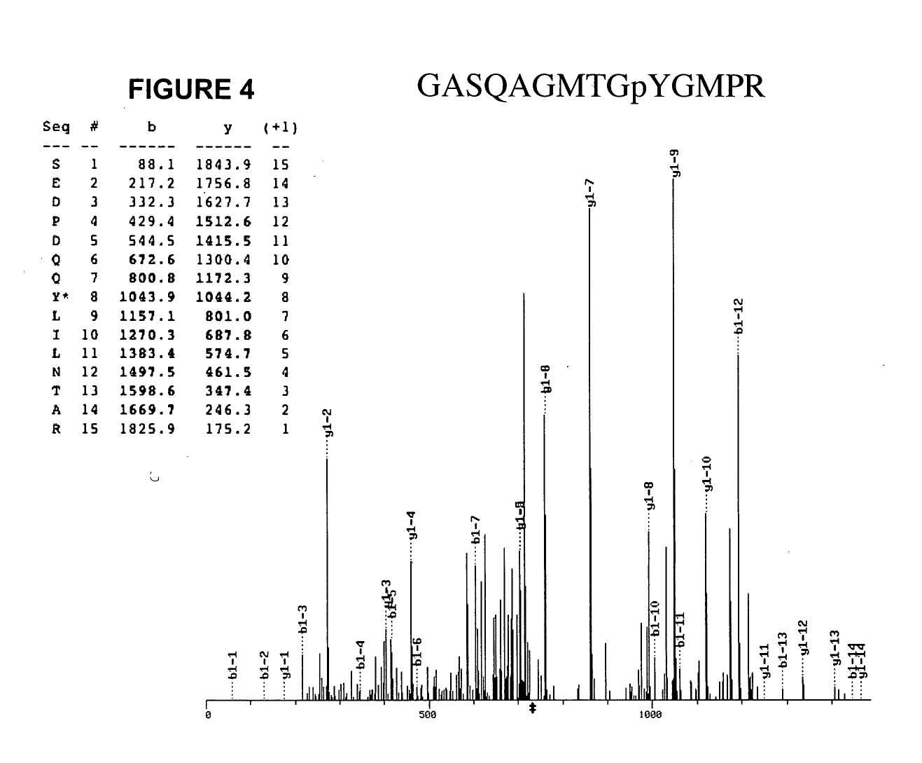

These phosphorylation sites correspond to numerous different parent proteins (the full sequences (human) of which are all publicly available in SwissProt database and their Accession numbers listed in Column B of Table 1/FIG. 2), each of which are have been linked to specific functions in the literature and thus may be organized into discrete protein type groups, for example adaptor/scaffold proteins, cytoskeletal proteins, protein kinases, and DNA binding proteins, etc. (see Column C of Table 1), the phosphorylation of which is relevant to signal transduction activity (e.g, underlying AML, CML, CLL, and ALL), as disclosed herein.

In part, the invention provides an isolated phosphorylation site-specific antibody that specifically binds a given target signaling protein/polypeptide only when phosphorylated (or not phosphorylated, respectively) at a particular tyrosine enumerated in Column D of Table 1/FIG. 2 comprised within the phosphorylatable peptide site sequence enumerated in corresponding Column E. In further part, the invention provides a heavy-isotope labeled peptide (AQUA peptide) for the detection and quantification of a given target signaling protein/polypeptide, the labeled peptide comprising a particular phosphorylatable peptide site/sequence enumerated in Column E of Table 1/FIG. 2 herein. For example, among the reagents provided by the invention is an isolated phosphorylation site-specific antibody that specifically binds the VAV1 adaptor/scaffold protein only when phosphorylated (or only when not phosphorylated) at tyrosine 791 (see Row 15 (and Columns D and E) of Table 1/FIG. 2). By way of further example, among the group of reagents provided by the invention is an AQUA peptide for the quantification of phosphorylated SLY adaptor/scaffold protein, the AQUA peptide comprising the phosphorylatable peptide sequence listed in Column E, Row 2, of Table 1/FIG. 2 (which encompasses the phosphorylatable tyrosine at position 116).

In one embodiment, the invention provides an isolated phosphorylation site-specific antibody that specifically binds a target signaling protein/polypeptide selected from Column A of Table 1 (Rows 2-384) only when phosphorylated at the tyrosine residue listed in corresponding Column D of Table 1, comprised within the phosphorylatable peptide sequence listed in corresponding Column E of Table 1 (SEQ ID NOs: 1-383), wherein said antibody does not bind said signaling protein when not phosphorylated at said tyrosine. In another embodiment, the invention provides an isolated phosphorylation site-specific antibody that specifically binds a target signaling protein/polypeptide selected from Column A of Table 1 only when not phosphorylated at the tyrosine residue listed in corresponding Column D of Table 1, comprised within the peptide sequence listed in corresponding Column E of Table 1 (SEQ ID NOs: 1-383), wherein said antibody does not bind said signaling protein when phosphorylated at said tyrosine. Such reagents enable the specific detection of phosphorylation (or non-phosphorylation) of a novel phosphorylatable site disclosed herein. The invention further provides immortalized cell lines producing such antibodies. In one embodiment, the immortalized cell line is a rabbit or mouse hybridoma.

In another embodiment, the invention provides a heavy-isotope labeled peptide (AQUA peptide) for the quantification of a target signaling protein/polypeptide selected from Column A of Table 1, said labeled peptide comprising the phosphorylatable peptide sequence listed in corresponding Column E of Table 1 (SEQ ID NOs: 1-383), which sequence comprises the phosphorylatable tyrosine listed in corresponding Column D of Table 1. In certain embodiments, the phosphorylatable tyrosine within the labeled peptide is phosphorylated, while in other embodiments, the phosphorylatable residue within the labeled peptide is not phosphorylated.

Reagents (antibodies and AQUA peptides) provided by the invention may conveniently be grouped by the type of target signaling protein/polypeptide in which a given phosphorylation site (for which reagents are provided) occurs. The protein types for each respective protein (in which a phosphorylation site has been discovered) are provided in Column C of Table 1/FIG. 2, and include: adaptor/scaffold proteins, adhesion/extracellular matrix protein, apoptosis proteins, calcium binding proteins, cell cycle regulation proteins, chaperone proteins, chromatin, DNA binding/repair/replication proteins, cytoskeletal proteins, endoplasmic reticulum or golgi proteins, enzyme proteins, G/regulator proteins, inhibitor proteins, motor/contractile proteins, phosphatase, protease, Ser/Thr protein kinases, protein kinase (Tyr)s, receptor/channel/cell suface proteins, RNA binding proteins, transcriptional regulators, tumor suppressor proteins, ubiquitan conjugating system proteins and proteins of unknown function. Each of these distinct protein groups is a subset of target signaling protein/polypeptide phosphorylation sites disclosed herein, and reagents for their detection/quantification may be considered a subset of reagents provided by the invention.

Subsets of the phosphorylation sites (and their corresponding proteins) disclosed herein are those occurring on the following protein types/groups listed in Column C of Table 1/FIG. 2 adaptor/scaffold proteins, calcium binding proteins, chromatin or DNA binding/repair/replication proteins, cytoskeletal proteins, enzyme proteins, protein kinases (Tyr), protein kinases (Ser/Thr), receptor/channel/transporter/cell suface proteins, transcriptional regulators and translational regulators. Accordingly, among subsets of reagents provided by the invention are isolated antibodies and AQUA peptides useful for the detection and/or quantification of the foregoing protein/phosphorylation site subsets.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

In one subset of embodiments, there is provided:

(i) An isolated phosphorylation site-specific antibody that specifically binds an cell cycle regulation protein selected from Column A, Rows 23-29, of Table 1 only when phosphorylated at the tyrosine listed in corresponding Column D, Rows 23-29, of Table 1, comprised within the phosphorylatable peptide sequence listed in corresponding Column E, Rows 23-29, of Table 1 (SEQ ID NOs: 22-28), wherein said antibody does not bind said protein when not phosphorylated at said tyrosine.
(ii) An equivalent antibody to (i) above that only binds the cell cycle regulation protein when not phosphorylated at the disclosed site (and does not bind the protein when it is phosphorylated at the site).
(iii) A heavy-isotope labeled peptide (AQUA peptide) for the quantification of a cell cycle regulation protein selected from Column A, Rows 23-29, said labeled peptide comprising the phosphorylatable peptide sequence listed in corresponding Column E, Rows 23-29, of Table 1 (SEQ ID NOs: 22-28), which sequence comprises the phosphorylatable tyrosine listed in corresponding Column D, Rows 23-29, of Table 1.

Among this subset of reagents, antibodies and AQUA peptides for the detection/quantification of the following cell cycle regulation protein phosphorylation sites are: TSG101 (Y32) and VCP (Y644) (see SEQ ID NOs: 25 and 27).

In a second subset of embodiments there is provided:

(i) An isolated phosphorylation site-specific antibody that specifically binds a chaperone protein selected from Column A, Rows 30-37, of Table 1 only when phosphorylated at the tyrosine listed in corresponding Column D, Rows 30-37, of Table 1, comprised within the phosphorylatable peptide sequence listed in corresponding Column E, Rows 30-37, of Table 1 (SEQ ID NOs: 29-36), wherein said antibody does not bind said protein when not phosphorylated at said tyrosine.
(ii) An equivalent antibody to (i) above that only binds the chaperone protein when not phosphorylated at the disclosed site (and does not bind the protein when it is phosphorylated at the site).
(iii) A heavy-isotope labeled peptide (AQUA peptide) for the quantification of a target signaling protein/polypeptide that is a chaperone protein selected from Column A, Rows 30-37, said labeled peptide comprising the phosphorylatable peptide sequence listed in corresponding Column E, Rows 30-37, of Table 1 (SEQ ID NOs: 29-36), which sequence comprises the phosphorylatable tyrosine listed in corresponding Column D, Rows 30-37, of Table 1.

Among this subset of reagents, antibodies and AQUA peptides for the detection/quantification of the following chaperone protein phosphorylation sites are: HSP90B (Y 192), STI1 (Y269) and TPR2 (Y317) (see SEQ ID NOs: 29, 30 and 36).

In another subset of embodiments there is provided:

(i) An isolated phosphorylation site-specific antibody that specifically binds a chromatin or DNA binding/repair/replication protein selected from Column A, Rows 38-55, of Table 1 only when phosphorylated at the tyrosine listed in corresponding Column D, Rows 38-55, of Table 1, comprised within the phosphorylatable peptide sequence listed in corresponding Column E, Rows 38-55, of Table 1 (SEQ ID NOs: 37-54), wherein said antibody does not bind said protein when not phosphorylated at said tyrosine.
(ii) An equivalent antibody to (i) above that only binds the chromatin or DNA binding/repair/replication protein when not phosphorylated at the disclosed site (and does not bind the protein when it is phosphorylated at the site).
(iii) A heavy-isotope labeled peptide (AQUA peptide) for the quantification of a target signaling protein/polypeptide that is a chromatin or DNA binding/repair/replication protein selected from Column A, Rows 38-55, said labeled peptide comprising the phosphorylatable peptide sequence listed in corresponding Column E, Rows 38-55, of Table 1 (SEQ ID NOs: 37-54), which sequence comprises the phosphorylatable tyrosine listed in corresponding Column D, Rows 38-55, of Table 1.

Among this subset of reagents, antibodies and AQUA peptides for the detection/quantification of the following chromatin or DNA binding/repair/replication protein phosphorylation sites are: TOP2B (Y230), TSN (Y210), TYMS (Y153) and WRN (Y849) (see SEQ ID NO's: 41, 43, 46 and 50).

In still another subset of embodiments there is provided:

(i) An isolated phosphorylation site-specific antibody that specifically binds a cytoskeletal protein selected from Column A, Rows 56-83, of Table 1 only when phosphorylated at the tyrosine listed in corresponding Column D, Rows 56-83, of Table 1, comprised within the phosphorylatable peptide sequence listed in corresponding Column E, Rows 56-83, of Table 1 (SEQ ID NOs: 55-82), wherein said antibody does not bind said protein when not phosphorylated at said tyrosine.
(ii) An equivalent antibody to (i) above that only binds the cytoskeletal protein when not phosphorylated at the disclosed site (and does not bind the protein when it is phosphorylated at the site).
(iii) A heavy-isotope labeled peptide (AQUA peptide) for the quantification of a signaling protein that is a cytoskeletal protein selected from Column A, Rows 56-83, said labeled peptide comprising the phosphorylatable peptide sequence listed in corresponding Column E, Rows 56-83, of Table 1 (SEQ ID NOs: 55-82), which sequence comprises the phosphorylatable tyrosine listed in corresponding Column D, Rows 56-83, of Table 1.

Among this subset of reagents, antibodies and AQUA peptides for the detection/quantification of the following cytoskeletal protein phosphorylation sites are:

SPTA1 (Y1538), SFTBN1 (Y1667), TAGLN3 (Y192), tubulin, beta-2 (Y51), VASP (Y16) and VIM (Y291) (see SEQ ID NOs: 56, 60, 65, 74, 78, and 80).

In still another subset of embodiments there is provided:

(i) An isolated phosphorylation site-specific antibody that specifically binds an enzyme protein selected from Column A, Rows 84-101, of Table 1 only when phosphorylated at the tyrosine listed in corresponding Column D, Rows 84-101, of Table 1, comprised within the phosphorylatable peptide sequence listed in corresponding Column E, Rows 84-101, of Table 1 (SEQ ID NOs: 83-100), wherein said antibody does not bind said protein when not phosphorylated at said tyrosine.
(ii) An equivalent antibody to (i) above that only binds the enzyme protein when not phosphorylated at the disclosed site (and does not bind the protein when it is phosphorylated at the site).
(iii) A heavy-isotope labeled peptide (AQUA peptide) for the quantification of a signaling protein that is a enzyme protein selected from Column A, Rows 84-101, said labeled peptide comprising the phosphorylatable peptide sequence listed in corresponding Column E, Rows 84-101, of Table 1 (SEQ ID NOs: 83-100), which sequence comprises the phosphorylatable tyrosine listed in corresponding Column D, Rows 84-101, of Table 1.

Among this subset of reagents, antibodies and AQUA peptides for the detection/quantification of the following enzyme protein phosphorylation sites are: TOP2A (Y328), TPH1 (Y401), TPI1 (Y48) and UAP1 (Y125) (see SEQ ID NOs: 86, 87, 89 and 91).

In still another subset of embodiments there is provided:

(i) An isolated phosphorylation site-specific antibody that specifically binds a protein kinase (Ser/Thr) selected from Column A, Rows 123-131, of Table 1 only when phosphorylated at the tyrosine listed in corresponding Column D, Rows 123-131, of Table 1, comprised within the phosphorylatable peptide sequence listed in corresponding Column E, Rows 123-131 of Table 1 (SEQ ID NOs: 122-130), wherein said antibody does not bind said protein when not phosphorylated at said tyrosine.
(ii) An equivalent antibody to (i) above that only binds protein kinase (Ser/Thr) when not phosphorylated at the disclosed site (and does not bind the protein when it is phosphorylated at the site).
(iii) A heavy-isotope labeled peptide (AQUA peptide) for the quantification of a signaling protein that is a protein kinase (Ser/Thr) selected from Column A, Rows 123-131, said labeled peptide comprising the phosphorylatable peptide sequence listed in corresponding Column E, Rows 123-131, of Table 1 (SEQ ID NOs: 122-130), which sequence comprises the phosphorylatable tyrosine listed in corresponding Column D, Rows 123-131, of Table 1.

Among this subset of reagents, antibodies and AQUA peptides for the detection/quantification of the following protein kinase (Ser/Thr) phosphorylation sites are: PKCD (Y374) and TRRAP (Y3497) (see SEQ ID NO: 122 and 128).

In yet another subset of embodiments, there is provided:

(i) An isolated phosphorylation site-specific antibody that specifically binds a protein kinase (Tyr) selected from Column A, Rows 132-141, of Table 1 only when phosphorylated at the tyrosine listed in corresponding Column D, Rows 132-141, of Table 1, comprised within the phosphorylatable peptide sequence listed in corresponding Column E, Rows 132-141, of Table 1 (SEQ ID NOs: 131-140), wherein said antibody does not bind said protein when not phosphorylated at said tyrosine.
(ii) An equivalent antibody to (i) above that only binds the protein kinase (Tyr) when not phosphorylated at the disclosed site (and does not bind the protein when it is phosphorylated at the site).
(iii) A heavy-isotope labeled peptide (AQUA peptide) for the quantification of a signaling protein that is a protein kinase (Tyr) selected from Column A, Rows 132-141, said labeled peptide comprising the phosphorylatable peptide sequence listed in corresponding Column E, Rows 132-141, of Table 1 (SEQ ID NOs: 131-140), which sequence comprises the phosphorylatable tyrosine listed in corresponding Column D, Rows 132-141, of Table 1.

Among this subset of reagents, antibodies and AQUA peptides for the detection/quantification of the following protein kinase (Tyr) phosphorylation sites are: Yes (Y146), TrkC (Y786) and Tyro3 (Y685) (see SEQ ID NOs: 131, 138 and 139).

In yet another subset of embodiments, there is provided:

(i) An isolated phosphorylation site-specific antibody that specifically binds an RNA binding protein selected from Column A, Rows 156-175, of Table 1 only when phosphorylated at the tyrosine listed in corresponding Column D, Rows 156-175, of Table 1, comprised within the phosphorylatable peptide sequence listed in corresponding Column E, Rows 156-175, of Table 1 (SEQ ID NOs: 155-174), wherein said antibody does not bind said protein when not phosphorylated at said tyrosine.
(ii) An equivalent antibody to (i) above that only binds the RNA binding protein when not phosphorylated at the disclosed site (and does not bind the protein when it is phosphorylated at the site).
(iii) A heavy-isotope labeled peptide (AQUA peptide) for the quantification of a signaling protein that is a RNA protein selected from Column A, Rows 156-175, said labeled peptide comprising the phosphorylatable peptide sequence listed in corresponding Column E, Rows 156-175, of Table 1 (SEQ ID NOs: 155-174), which sequence comprises the phosphorylatable tyrosine listed in corresponding Column D, Rows 156-175, of Table 1.

Among this subset of reagents, antibodies and AQUA peptides for the detection/quantification of the following RNA protein phosphorylation sites are: SNRPN (Y15), UPF2 (Y974) and UPF3B (Y160) (see SEQ ID NOs: 156, 169 and 170).

In yet another subset of embodiments, there is provided:

(i) An isolated phosphorylation site-specific antibody that specifically binds a transcriptional regulator selected from Column A, Rows 176-231, of Table 1 only when phosphorylated at the tyrosine listed in corresponding Column D, Rows 176-231, of Table 1, comprised within the phosphorylatable peptide sequence listed in corresponding Column E, Rows 176-231, of Table 1 (SEQ ID NOs: 175-230), wherein said antibody does not bind said protein when not phosphorylated at said tyrosine.
(ii) An equivalent antibody to (i) above that only binds the transcriptional regulator when not phosphorylated at the disclosed site (and does not bind the protein when it is phosphorylated at the site).
(iii) A heavy-isotope labeled peptide (AQUA peptide) for the quantification of a target signaling protein/polypeptide that is a transcriptional regulator selected from Column A, Rows 176-231, said labeled peptide comprising the phosphorylatable peptide sequence listed in corresponding Column E, Rows 176-231, of Table 1 (SEQ ID NOs: 175-230), which sequence comprises the phosphorylatable tyrosine listed in corresponding Column D, Rows 176-231, of Table 1.

Among this subset of reagents, antibodies and AQUA peptides for the detection/quantification of the following transcriptional regulator phosphorylation sites are:

### SPT5 (Y140), SSB (Y23), SSRP1 (Y452), STAT3 (Y674), STAT5B (Y171), TAF172 (Y415), TCF12 (Y82), TEL (Y401) and TFIIF (Y124) (see SEQ ID NO: 178, 185, 186, 190, 192, 194, 201, 211 and 213).

In still another subset of embodiments, there is provided:

(i) An isolated phosphorylation site-specific antibody that specifically binds a translational regulator selected from Column A, Rows 234-249, of Table 1 only when phosphorylated at the tyrosine listed in corresponding Column D, Rows 234-249, of Table 1, comprised within the phosphorylatable peptide sequence listed in corresponding Column E, Rows 234-249, of Table 1 (SEQ ID NOs: 233-248), wherein said antibody does not bind said protein when not phosphorylated at said tyrosine.
(ii) An equivalent antibody to (i) above that only binds the translational regulator when not phosphorylated at the disclosed site (and does not bind the protein when it is phosphorylated at the site).
(iii) A heavy-isotope labeled peptide (AQUA peptide) for the quantification of a signaling protein that translational regulator selected from Column A, Rows 234-249, said labeled peptide comprising the phosphorylatable peptide sequence listed in corresponding Column E, Rows 234-249, of Table 1 (SEQ ID NOs: 233-248), which sequence comprises the phosphorylatable tyrosine listed in corresponding Column D, Rows 234-249, of Table 1.

Among this subset of reagents, antibodies and AQUA peptides for the detection/quantification of the following a translational regulator phosphorylation sites are: USP14 (Y417) and USP20 (Y227) (see SEQ ID NO: 243 and 245).

In yet a further subset of embodiments, there is provided:

(i) An isolated phosphorylation site-specific antibody that specifically binds SPTAN1 (Y976), (Column A, Row 5, of Table 1) only when phosphorylated at the tyrosine listed in corresponding Column D of Table 1), said tyrosine comprised within the phosphorylatable peptide sequence listed in corresponding Column E of Table 1 (SEQ ID NO: 4), wherein said antibody does not bind said protein when not phosphorylated at said tyrosine.
(ii) An equivalent antibody to (i) above that only binds SPTAN1 (Y976) (Column A, Row 5 of Table 1) when not phosphorylated at the disclosed site (and does not bind the protein when it is phosphorylated at the site).
(iii) A heavy-isotope labeled peptide (AQUA peptide) for the quantification of SPTAN1 (Y976) (Column A, Row 5 of Table 1), said labeled peptide comprising the phosphorylatable peptide sequence listed in corresponding Column E of Table 1 (SEQ ID NO: 4), which sequence comprises the phosphorylatable tyrosine listed in corresponding Column D, Row 5 of Table 1.

The invention also provides an immortalized cell line producing an antibody of the invention, for example, a cell line producing an antibody within any of the foregoing subsets of antibodies. In an embodiment, the immortalized cell line is a rabbit hybridoma or a mouse hybridoma.

In other embodiments, a heavy-isotope labeled peptide (AQUA peptide) of the invention (for example, an AQUA peptide within any of the foregoing subsets of AQUA peptides) comprises a disclosed site sequence wherein the phosphorylatable tyrosine is phosphorylated. In yet other embodiments, a heavy-isotope labeled peptide of the invention comprises a disclosed site sequence wherein the phosphorylatable tyrosine is not phosphorylated.

The foregoing subsets of reagents of the invention should not be construed as limiting the scope of the invention, which, as noted above, includes reagents for the detection and/or quantification of disclosed phosphorylation sites on any of the other protein type/group subsets (each a subset) listed in Column C of Table 1/FIG. 2.

Also provided by the invention are methods for detecting or quantifying a target signaling protein/polypeptide that is tyrosine phosphorylated, said method comprising the step of utilizing one or more of the above-described reagents of the invention to detect or quantify one or more target Signaling Protein(s)/Polypeptide(s) selected from Column A of Table 1 only when phosphorylated at the tyrosine listed in corresponding Column D of Table 1. In certain embodiments of the methods of the invention, the reagents comprise a subset of reagents as described above. The antibodies according to the invention maybe used in standard (e.g., ELISA or conventional cytometric assays). The invention thus, provides compositions and methods for the detection and/or quantitation of a given target signaling protein or polypeptide in a sample, by contacting the sample and a control sample with one or more antibody of the invention under conditions favoring the binding and thus formation of the complex of the antibody with the protein or peptide. The formation of the complex is then detected according to methods well established and known in the art.

Also provided by the invention is a method for obtaining a phosphorylation profile of a certain protein type or group, for example adaptor/scaffold proteins or cell cycle regulation proteins (Rows 2-20 and Rows 23-29, respectively, of Table 1), that is phosphorylated in a disease signaling pathway, said method comprising the step of utilizing one or more isolated antibody that specifically binds the protein group selected from Column A of Table 1 only when phosphorylated at the tyrosine listed in corresponding Column D, of Table 1, comprised within the phosphorylation site sequence listed in corresponding Column E, to detect the phosphorylation of one or more of said protein group, thereby obtaining a phosphorylation profile for said protein group.

The invention further contemplates compositions, foremost pharmaceutical compositions, containing onr or a more antibody according to the invention formulated together with a pharmaceutically acceptable carrier. One of skill will appreciate that in certain instances the composition of the invention may further comprise other pharmaceutically active moieties. The compounds according to the invention are optionally formulated in a pharmaceutically acceptable vehicle with any of the well-known pharmaceutically acceptable carriers, including diluents and excipients (see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention), as well any other pharmacologically active ingredient useful for the treatment of the symptom/condition being treated.

The invention also provides methods of treating a mammal comprising the step of administering such a mammal a therapeutically effective amount of a composition according to the invention.

As used herein, by “treating” is meant reducing, preventing, and/or reversing the symptoms in the individual to which a compound of the invention has been administered, as compared to the symptoms of an individual not being treated according to the invention. A practitioner will appreciate that the compounds, compositions, and methods described herein are to be used in concomitance with continuous clinical evaluations by a skilled practitioner (physician or veterinarian) to determine subsequent therapy. Hence, following treatment the practitioners will evaluate any improvement in the treatment of the pulmonary inflammation according to standard methodologies. Such evaluation will aid and inform in evaluating whether to increase, reduce or continue a particular treatment dose, mode of administration, etc. The term “therapeutic composition” refers to any compounds administered to treat or prevent a disease. It will be understood that the subject to which a compound (e.g., an antibody) of the invention is administered need not suffer from a specific traumatic state. Indeed, the compounds (e.g., antibodies) of the invention may be administered prophylactically, prior to any development of symptoms. The term “therapeutic,” “therapeutically,” and permutations of these terms are used to encompass therapeutic, palliative as well as prophylactic uses. Hence, as used herein, by “treating or alleviating the symptoms” is meant reducing, preventing, and/or reversing the symptoms of the individual to which a compound of the invention has been administered, as compared to the symptoms of an individual receiving no such administration.

The term “therapeutically effective amount” is used to denote treatments at dosages effective to achieve the therapeutic result sought. Furthermore, one of skill will appreciate that the therapeutically effective amount of the compound of the invention may be lowered or increased by fine tuning and/or by administering more than one compound of the invention, or by administering a compound of the invention with another compound. See, for example, Meiner, C. L., “Clinical Trials: Design, Conduct, and Analysis,” Monographs in Epidemiology and Biostatistics, Vol. 8 Oxford University Press, USA (1986). The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect.

TABLE 1
Phosphorylation Sites

| | A | | | D | | |
|---|---|---|---|---|---|---|
| | Protein | B | C | Phospho- | E | H |
| 1 | Name | Accession No. | Protein Type | Residue | Phosphorylation Site Sequence | SEQ ID NO |
| 2 | SLY | NP_061863.1 | Adaptor/scaffold | Y116 | ALSEEMADTLEEGSASPTSPDySLDSPGPEK | SEQ ID NO. 1 |
| 3 | SPTAN1 | NP_003118.1 | Adaptor/scaffold | Y601 | TATDEAyKDPSNLQGK | SEQ ID NO. 2 |
| 4 | SPTAN1 | NP_003118.1 | Adaptor/scaffold | Y942 | KHEALMSDLSAyGSSIQALR | SEQ ID NO. 3 |
| 5 | SPTAN1 | NP_003118.1 | Adaptor/scaffold | Y976 | QQVAPTDDETGKELVLALyDYQEKSPR | SEQ ID NO. 4 |
| 6 | SPTAN1 | NP_003118.1 | Adaptor/scaffold | Y1020 | QGFVPAAyVK | SEQ ID NO. 5 |
| 7 | SPTAN1 | NP_003118.1 | Adaptor/scaffold | Y1579 | LQTASDESyKDPTNIQSK | SEQ ID NO. 6 |
| 8 | SYNE2 | NP_055995.4 | Adaptor/scaffold | Y3372 | KMEEDIyTNLSK | SEQ ID NO. 7 |
| 9 | synergin, | NP_009178.3 | Adaptor/scaffold | Y514 | ALPSMDKyAVFK | SEQ ID NO. 8 |
| | gamma | | | | | |
| 10 | syntenin | NP_005616.2 | Adaptor/scaffold | Y56 | LYPELSQyMGLSLNEEEIR | SEQ ID NO. 9 |
| 11 | TRAF3IP3 | NP_079504.1 | Adaptor/scaffold | Y165 | AEGPTIKNDASQQTNyGVAVLDKEIIQLSDYLK | SEQ ID NO. 10 |
| 12 | TRAF3IP3 | NP_079504.1 | Adaptor/scaffold | Y234 | SLENQLyTCTQKYSPWGMK | SEQ ID NO. 11 |
| 13 | TRAF3IP3 | NP_079504.1 | Adaptor/scaffold | Y240 | SLENQLYTCTQKySPWGMK | SEQ ID NO. 12 |
| 14 | TRAF3IP3 | NP_079504.1 | Adaptor/scaffold | Y302 | SQyEALKEDWR | SEQ ID NO. 13 |
| 15 | VAV1 | NP_005419.2 | Adaptor/scaffold | Y791 | ARyDFCAR | SEQ ID NO. 14 |
| 16 | VAV3 | NP_006104.4 | Adaptor/scaffold | Y508 | WLEQFEMALSNIRPDyADSNFHDFK | SEQ ID NO. 15 |
| 17 | VAV3 | NP_006104.4 | Adaptor/scaffold | Y797 | ARyDFCAR | SEQ ID NO. 16 |
| 18 | WAC | NP_057712.2 | Adaptor/scaffold | Y25 | RGDSQPyQALK | SEQ ID NO. 17 |
| 19 | ZO1 | NP_003248.3 | Adaptor/scaffold | Y830 | ADGATSDDLDLHDDRLSYLSAPGSEySMYSTDSR | SEQ ID NO. 18 |
| 20 | ZO1 | NP_003248.3 | Adaptor/scaffold | Y1324 | TLyRIPEPQKPQLKPPEDIVR | SEQ ID NO. 19 |
| 21 | URP2 | NP_113659.3 | Adhesion or | Y162 | EKEPEEELyDLSKVVLAGGVAPALFR | SEQ ID NO. 20 |
| | | | extracellular | | | |
| | | | matrix protein | | | |
| 22 | zyxin | NP_003452.1 | Adhesion or | Y20 | PSPAISVSVSAPAFyAPQKK | SEQ ID NO. 21 |
| | | | extracellular | | | |
| | | | matrix protein | | | |
| 23 | SUGT1 | NP_006695.1 | Cell cycle | Y251 | NLYPSSSPyTR | SEQ ID NO. 22 |
| | | | regulation | | | |
| 24 | TPX2 | NP_036244.2 | Cell cycle | Y81 | ANLQQAIVTPLKPVDNTyYK | SEQ ID NO. 23 |
| | | | regulation | | | |
| 25 | TSG101 | NP_006283.1 | Cell cycle | Y15 | yKYRDLTVRETVNVITLYK | SEQ ID NO. 24 |
| | | | regulation | | | |
| 26 | TSG101 | NP_006283.1 | Cell cycle | Y32 | YKYRDLTVRETVNVITLyK | SEQ ID NO. 25 |
| | | | regulation | | | |
| 27 | VCP | NP_009057.1 | Cell cycle | Y244 | GILLyGPPGTGK | SEQ ID NO. 26 |
| | | | regulation | | | |
| 28 | VCP | NP_009057.1 | Cell cycle | Y644 | LDQLIyIPLPDEK | SEQ ID NO. 27 |
| | | | regulation | | | |
| 29 | ZW10 | NP_004715.1 | Cell cycle | Y349 | NCLVySIPTNSSK | SEQ ID NO. 28 |
| | | | regulation | | | |
| 30 | HSP90B | NP_031381.2 | Chaperone | Y192 | VILHLKEDQTEyLEER | SEQ ID NO. 29 |
| 31 | STI1 | NP_006810.1 | Chaperone | Y269 | ELDPTNMTYITNQAAVyFEKGDYNK | SEQ ID NO. 30 |
| 32 | STI1 | NP_006810.1 | Chaperone | Y275 | ELDPTNMTYITNQAAVYFEKGDyNK | SEQ ID NO. 31 |
| 33 | STI1 | NP_006810.1 | Chaperone | Y444 | AAALEAMKDyTK | SEQ ID NO. 32 |
| 34 | TBCA | NP_004598.1 | Chaperone | Y48 | MRAEDGENyDIKK | SEQ ID NO. 33 |
| 35 | TBCA | NP_004598.1 | Chaperone | Y94 | ILENEKDLEEAEEyKEAR | SEQ ID NO. 34 |
| 36 | TPR2 | NP_003306.1 | Chaperone | Y41 | KDYNEAYNYyTK | SEQ ID NO. 35 |
| 37 | TPR2 | NP_003306.1 | Chaperone | Y317 | KLDDAIEDCTNAVKLDDTyIK | SEQ ID NO. 36 |
| 38 | Smc5 | NP_055925.1 | Chromatin, DNA- | Y282 | RPWVEYENVRQEyEEVK | SEQ ID NO. 37 |
| | | | binding, DNA | | | |
| | | | repair or DNA | | | |
| | | | replication | | | |
| | | | protein | | | |
| 39 | SON | NP_115571.1 | Chromatin, DNA- | Y1037 | SMMSAyER | SEQ ID NO. 38 |
| | | | binding, DNA | | | |
| | | | repair or DNA | | | |
| | | | replication | | | |
| | | | protein | | | |
| 40 | SON | NP_115571.1 | Chromatin, DNA- | Y1096 | SMMSSySAADR | SEQ ID NO. 39 |
| | | | binding, DNA | | | |
| | | | repair or DNA | | | |
| | | | replication | | | |
| | | | protein | | | |
| 41 | SON | NP_115571.1 | Chromatin, DNA- | Y2192 | EADSVyGEWVPVEK | SEQ ID NO. 40 |
| | | | binding, DNA | | | |
| | | | repair or DNA | | | |
| | | | replication | | | |
| | | | protein | | | |
| 42 | TOP2B | NP_001059.2 | Chromatin, DNA- | Y230 | IKHFDGEDyTCITFQPDLSK | SEQ ID NO. 41 |
| | | | binding, DNA | | | |
| | | | repair or DNA | | | |
| | | | replication | | | |
| | | | protein | | | |
| 43 | TOP2B | NP_001059.2 | Chromatin, DNA- | Y344 | HVDyVVDQVVGK | SEQ ID NO. 42 |
| | | | binding, DNA | | | |
| | | | repair or DNA | | | |
| | | | replication | | | |
| | | | protein | | | |
| 44 | TSN | NP_004613.1 | Chromatin, DNA- | Y210 | KVEEVVyDLSIR | SEQ ID NO. 43 |
| | | | binding, DNA | | | |
| | | | repair or DNA | | | |
| | | | replication | | | |
| | | | protein | | | |
| 45 | TSNAX | NP_005990.1 | Chromatin, DNA- | Y237 | QVYDGFSFIGNTGPyEVSKK | SEQ ID NO. 44 |
| | | | binding, DNA | | | |
| | | | repair or DNA | | | |
| | | | replication | | | |
| | | | protein | | | |
| 46 | TTF2 | NP_003585.3 | Chromatin, DNA- | Y285 | GGPLNKEyTNWEAK | SEQ ID NO. 45 |
| | | | binding, DNA | | | |
| | | | repair or DNA | | | |
| | | | replication | | | |
| | | | protein | | | |
| 47 | TYMS | NP_001062.1 | Chromatin, DNA- | Y153 | DMESDySGQGVDQLQR | SEQ ID NO. 46 |
| | | | binding, DNA | | | |
| | | | repair or DNA | | | |
| | | | replication | | | |
| | | | protein | | | |
| 48 | UKp68 | NP_079100.2 | Chromatin, DNA- | Y321 | KFNHDGEEEEEDDDyGSR | SEQ ID NO. 47 |
| | | | binding, DNA | | | |
| | | | repair or DNA | | | |
| | | | replication | | | |
| | | | protein | | | |
| 49 | WBP11 | NP_057396.1 | Chromatin, DNA- | Y236 | RRDEDMLySPELAQR | SEQ ID NO. 48 |
| | | | binding, DNA | | | |
| | | | repair or DNA | | | |
| | | | replication | | | |
| | | | protein | | | |
| 50 | WBP11 | NP_057396.1 | Chromatin, DNA- | Y630 | DDVyEAFMK | SEQ ID NO. 49 |
| | | | binding, DNA | | | |
| | | | repair or DNA | | | |
| | | | replication | | | |
| | | | protein | | | |
| 51 | WRN | NP_000544.2 | Chromatin, DNA- | Y849 | DMESYyQEIGR | SEQ ID NO. 50 |
| | | | binding, DNA | | | |
| | | | repair or DNA | | | |
| | | | replication | | | |
| | | | protein | | | |
| 52 | XPG | NP_000114.2 | Chromatin, DNA- | Y289 | RVVSEDTSHyILIK | SEQ ID NO. 51 |
| | | | binding, DNA | | | |
| | | | repair or DNA | | | |
| | | | replication | | | |
| | | | protein | | | |
| 53 | ZAP | NP_064504.2 | Chromatin, DNA- | Y299 | KFTyLGSQDR | SEQ ID NO. 52 |
| | | | binding, DNA | | | |
| | | | repair or DNA | | | |
| | | | replication | | | |
| | | | protein | | | |
| 54 | ZAP | NP_064504.2 | Chromatin, DNA- | Y348 | FLENGSQEDLLHGNPGSTyLASNSTSAPNWK | SEQ ID NO. 53 |
| | | | binding, DNA | | | |
| | | | repair or DNA | | | |
| | | | replication | | | |
| | | | protein | | | |
| 55 | ZAP | NP_064504.2 | Chromatin, DNA- | Y826 | DAIySHKNCPYDAK | SEQ ID NO. 54 |
| | | | binding, DNA | | | |
| | | | repair or DNA | | | |
| | | | replication | | | |
| | | | protein | | | |
| 56 | SPIRE1 | NP_064533.2 | Cytoskeletal | Y405 | QVLVKAELEKyQQYK | SEQ ID NO. 55 |
| | | | protein | | | |
| 57 | SPTA1 | NP_003117.2 | Cytoskeletal | Y1538 | DATNIQRKyLK | SEQ ID NO. 56 |
| | | | protein | | | |
| 58 | SPTA1 | NP_003117.2 | Cytoskeletal | Y2333 | KGyVSLEDYTAFLIDKESENIK | SEQ ID NO. 57 |
| | | | protein | | | |
| 59 | SPTBN1 | NP_842565.2 | Cytoskeletal | Y66 | ITDLyTDLR | SEQ ID NO. 58 |
| | | | protein | | | |
| 60 | SPTBN1 | NP_842565.2 | Cytoskeletal | Y1290 | MLTAQDMSyDEAR | SEQ ID NO. 59 |
| | | | protein | | | |
| 61 | SPTBN1 | NP_842565.2 | Cytoskeletal | Y1667 | VDKLyAGLKDLAEER | SEQ ID NO. 60 |
| | | | protein | | | |
| 62 | SPTBN1 | NP_842565.2 | Cytoskeletal | Y17 | TSSISGPLSPAyTGQVPYNYNQLEGR | SEQ ID NO. 61 |
| | iso 2 | | protein | | | |
| 63 | SPTBN1 | NP_8425652 | Cytoskeletal | Y23 | TSSISGPLSPAYTGQVPyNYNQLEGR | SEQ ID NO. 62 |
| | iso 2 | | protein | | | |
| 64 | SPTBN2 | NP_008877.1 | Cytoskeletal | Y1726 | EVVAASHELGQDyEHVTMLR | SEQ ID NO. 63 |
| | | | protein | | | |
| 65 | SSFA2 | NP_006742.2 | Cytoskeletal | Y930 | NSLQNLSQyPMMR | SEQ ID NO. 64 |
| | | | protein | | | |
| 66 | TAGLN3 | NP_037391.2 | Cytoskeletal | Y192 | GASQAGMTGyGMPR | SEQ ID NO. 65 |
| | | | protein | | | |
| 67 | talin 1 | NP_006280.2 | Cytoskeletal | Y71 | KGIWLEAGKALDYyMLR | SEQ ID NO. 66 |
| | | | protein | | | |
| 68 | tau | NP_005901.2 | Cytoskeletal | Y197 | SGySSPGSPGTPGSR | SEQ ID NO. 67 |
| | | | protein | | | |
| 69 | tau | NP_005901.2 | Cytoskeletal | Y310 | VQIVyKPVDLSK | SEQ ID NO. 68 |
| | | | protein | | | |
| 70 | tensin 2 | NP_056134.2 | Cytoskeletal | Y255 | LGVIVSAyMHYSK | SEQ ID NO. 69 |
| | | | protein | | | |
| 71 | tensin 2 | NP_056134.2 | Cytoskeletal | Y258 | LGVIVSAYMHySK | SEQ ID NO. 70 |
| | | | protein | | | |
| 72 | TES | NP_056456.1 | Cytoskeletal | Y237 | RTQYSCyCCK | SEQ ID NO. 71 |
| | | | protein | | | |
| 73 | TMOD3 | NP_055362.1 | Cytoskeletal | Y86 | DREDyVPYTGEK | SEQ ID NO. 72 |
| | | | protein | | | |
| 74 | TPM4 | NP_003281.1 | Cytoskeletal | Y126 | HIAEEADRKyEEVAR | SEQ ID NO. 73 |
| | | | protein | | | |
| 75 | tubulin, | NP_006079.1 | Cytoskeletal | Y51 | INVYyNEATGGK | SEQ ID NO. 74 |
| | beta-2 | | protein | | | |
| 76 | utrophin | NP_009055.2 | Cytoskeletal | Y1757 | KLLIAQEPLyQC | SEQ ID NO. 75 |
| | | | protein | | | |
| 77 | utrophin | NP_009055.2 | Cytoskeletal | Y3111 | LHyPMVEYCIPTTSGEDVRDFTK | SEQ ID NO. 76 |
| | | | protein | | | |
| 78 | utrophin | NP_009055.2 | Cytoskeletal | Y3116 | LHYPMVEyCIPTTSGEDVR | SEQ ID NO. 77 |
| | | | protein | | | |
| 79 | VASP | NP_003361.1 | Cytoskeletal | Y16 | ATVMLyDDGNKR | SEQ ID NO. 78 |
| | | | protein | | | |
| 80 | VASP | NP_003361.1 | Cytoskeletal | Y341 | SSSSVTTSETQPCTPSSSDySDLQR | SEQ ID NO. 79 |
| | | | protein | | | |
| 81 | VIM | NP_003371.2 | Cytoskeletal | Y291 | NLQEAEEWyK | SEQ ID NO. 80 |
| | | | protein | | | |
| 82 | WAVE1 | NP_003922.1 | Cytoskeletal | Y156 | FYTNPSyFFDLWK | SEQ ID NO. 81 |
| | | | protein | | | |
| 83 | WDR1 | NP_059830.1 | Cytoskeletal | Y98 | YEyQPFAGK | SEQ ID NO. 82 |
| | | | protein | | | |
| 84 | SLC27A2 | NP_003636.1 | Enzyme, misc. | Y602 | MyVPMTEDIYNAISAK | SEQ ID NO. 83 |
| 85 | SLC27A2 | NP_003636.1 | Enzyme, misc. | Y610 | MYVPMTEDIyNAISAK | SEQ ID NO. 84 |
| 86 | TKT | NP_001055.1 | Enzyme, misc. | Y202 | LGQSDPAPLQHQMDIyQK | SEQ ID NO. 85 |
| 87 | TOP2A | NP_001058.2 | Enzyme, misc. | Y328 | HVDyVADQIVTK | SEQ ID NO. 86 |
| 88 | TPH1 | NP_004170.1 | Enzyme, misc. | Y401 | yNPYTRSIQILK | SEQ ID NO. 87 |
| 89 | TPH1 | NP_004170.1 | Enzyme, misc. | Y404 | YNPyTRSIQILK | SEQ ID NO. 88 |
| 90 | TPI1 | NP_000356.1 | Enzyme, misc. | Y48 | VPADTEVVCAPPTAyIDFAR | SEQ ID NO. 89 |
| 91 | TPI1 | NP_000356.1 | Enzyme, misc. | Y165 | VVLAyEPVWAIGTGK | SEQ ID NO. 90 |
| 92 | UAP1 | NP_003106.2 | Enzyme, misc. | Y125 | GMyDVGLPSR | SEQ ID NO. 91 |
| 93 | UAP1 | NP_003106.2 | Enzyme, misc. | Y299 | TNPTEPVGVVCRVDGVyQVVEYSEISLATAQK | SEQ ID NO. 92 |
| 94 | UGP1 | NP_006750.3 | Enzyme, misc. | Y298 | GGTLTQyEGKLR | SEQ ID NO. 93 |
| 95 | UMPS | NP_000364.1 | Enzyme, misc. | Y16 | AALGPLVTGLyDVQAFK | SEQ ID NO. 94 |
| 96 | UROD | NP_000365.3 | Enzyme, misc. | Y342 | YIANLGHGLyPDMDPEHVGAFVDAVHK | SEQ ID NO. 95 |
| 97 | UROD | NP_000365.3 | Enzyme, misc. | Y30 | AAWGEETDyTPVWCMR | SEQ ID NO. 96 |
| 98 | VARS2 | NP_006286.1 | Enzyme, misc. | Y878 | SLGNVIDPLDVIyGISLQGLHNQLLNSNLDPSEV | SEQ ID NO. 97 |
| | | | | | EKAK | |
| 99 | WRNIP1 | NP_064520.2 | Enzyme, misc. | Y500 | SHILYDRAGEEHyNCISALHK | SEQ ID NO. 98 |
| 100 | ZDHHC3 | NP_057682.1 | Enzyme, misc. | Y127 | EFIESLQLKPGQVVyK | SEQ ID NO. 99 |
| 101 | ZDHHC3 | NP_057682.1 | Enzyme, misc. | Y323 | AVFGHPFSLGWASPFATPDQGKADPyQYVV | SEQ ID NO. 100 |
| 102 | SRGAP2 | NP_056141.2 | G protein or | Y161 | VLNELySVMK | SEQ ID NO. 101 |
| | | | regulator | | | |
| 103 | SWAP-70 | NP_055870.2 | G protein or | Y141 | YPLIIVSEEIEyLLK | SEQ ID NO. 102 |
| | | | regulator | | | |
| 104 | TD-60 | NP_061185.1 | G protein or | Y276 | GNLySFGCPEYGQLGHNSDGK | SEQ ID NO. 103 |
| | | | regulator | | | |
| 105 | TD-60 | NP_061185.1 | G protein or | Y359 | VFSWGFGGyGR | SEQ ID NO. 104 |
| | | | regulator | | | |
| 106 | VAV1 | NP_005419.2 | G protein or | Y841 | VGWFPANYVEEDySEYC | SEQ ID NO. 105 |
| | | | regulator | | | |
| 107 | SOCS7 | NP_055413.1 | Inhibitor protein | Y507 | LyPVSRF | SEQ ID NO. 106 |
| 108 | SPRED1 | NP_689807.1 | Inhibitor protein | Y82 | MVVLECMLKKDLIyNK | SEQ ID NO. 107 |
| 109 | THBS1 | NP_003237.2 | Inhibitor protein | Y817 | DNCQYVyNVDQR | SEQ ID NO. 108 |
| 110 | TIMM44 | NP_006342.2 | Mitochondrial | Y435 | DQDELNPyAAWR | SEQ ID NO. 109 |
| | | | protein | | | |
| 111 | TOP1MT | NP_443195.1 | Mitochondrial | Y144 | yFVDKAAARKVLSR | SEQ ID NO. 110 |
| | | | protein | | | |
| 112 | TBP7 | NP_006494.1 | Protease | Y41 | AQDEIPALSVSRPQTGLSFLGPEPEDLEDLySR | SEQ ID NO. 111 |
| 113 | TBP7 | NP_006494.1 | Protease | Y205 | GVLMyGPPGCGK | SEQ ID NO. 112 |
| 114 | TBP7 | NP_006494.1 | Protease | Y417 | KDEQEHEFyK | SEQ ID NO. 113 |
| 115 | TPP2 | NP_003282.1 | Protease | Y645 | VNESSHyDLAFTDVHFKPGQIR | SEQ ID NO. 114 |
| 116 | TPP2 | NP_003282.1 | Protease | Y1052 | LDSSCIYNELKETYPNyLPLYVAR | SEQ ID NO. 115 |
| 117 | USP24 | XP_371254.5 | Protease | Y1697 | GEVLEGSNAyYCEK | SEQ ID NO. 116 |
| 118 | USP24 | XP_371254.5 | Protease | Y1858 | VyDQTNPYTDVR | SEQ ID NO. 117 |
| 119 | XPNPEP1 | NP_065116.2 | Protease | Y312 | CCMPyTPICIAK | SEQ ID NO. 118 |
| 120 | TIF1- | NP_005753.1 | Protein kinase | Y517 | VFPGSTTEDyNLIVIER | SEQ ID NO. 119 |
| | beta | | | | | |
| 121 | TTK | NP_003309.2 | Protein kinase, | Y833 | TLyEHYSGGESHNSSSSK | SEQ ID NO. 120 |
| | | | dual-specificity | | | |
| 122 | TTK | NP_003309.2 | Protein kinase, | Y836 | TLYEHySGGESHNSSSSK | SEQ ID NO. 121 |
| | | | dual-specificity | | | |
| 123 | PKCD | NP_006245.2 | Protein kinase, | Y374 | GRGEyFAIK | SEQ ID NO. 122 |
| | | | Ser/Thr (non- | | | |
| | | | receptor) | | | |
| 124 | TAO1 | NP_065842.1 | Protein kinase, | Y43 | EIGHGSFGAVyFAR | SEQ ID NO. 123 |
| | | | Ser/Thr (non- | | | |
| | | | receptor) | | | |
| 125 | TAO2 | NP_004774.1 | Protein kinase, | Y61 | KMSySGKQSNEK | SEQ ID NO. 124 |
| | | | Ser/Thr (non- | | | |
| | | | receptor) | | | |
| 126 | Titin | NP_003310.3 | Protein kinase, | Y4912 | PIEDVTIyEK | SEQ ID NO. 125 |
| | | | Ser/Thr (non- | | | |
| | | | receptor) | | | |
| 127 | Titin | NP_003310.3 | Protein kinase, | Y15976 | ITGyIVEK | SEQ ID NO. 126 |
| | | | Ser/Thr (non- | | | |
| | | | receptor) | | | |
| 128 | TRRAP | NP_003487.1 | Protein kinase, | Y862 | TLELCVDNLQPDFLyDHIQPVR | SEQ ID NO. 127 |
| | | | Ser/Thr (non- | | | |
| | | | receptor) | | | |
| 129 | TRRAP | NP_003487.1 | Protein kinase, | Y3497 | GHNGKIyPYLVMNDACLTESR | SEQ ID NO. 128 |
| | | | Ser/Thr (non- | | | |
| | | | receptor) | | | |
| 130 | WNK1 | NP_061852.1 | Protein kinase, | Y1215 | DDyGFSGSQK | SEQ ID NO. 129 |
| | | | Ser/Thr(non- | | | |
| | | | receptor) | | | |
| 131 | YSK1 | NP_006365.2 | Protein kinase, | Y35 | IGKGSFGEVyK | SEQ ID NO. 130 |
| | | | Ser/Thr (non- | | | |
| | | | receptor) | | | |
| 132 | Yes | NP_005424.1 | Protein kinase, | Y146 | NGYIPSNyVAPADSIQAEEWYFGK | SEQ ID NO. 131 |
| | | | Tyr (non- | | | |
| | | | receptor) | | | |
| 133 | ZAP70 | NP_001070.2 | Protein kinase, | Y178 | KLySGAQTDGK | SEQ ID NO. 132 |
| | | | Tyr (non- | | | |
| | | | receptor) | | | |
| 134 | ZAP70 | NP_001070.2 | Protein kinase, | Y204 | KEQGTYALSLIyGK | SEQ ID NO. 133 |
| | | | Tyr (non- | | | |
| | | | receptor) | | | |
| 135 | ZAP70 | NP_001070.2 | Protein kinase, | Y597 | MRACyYSLASK | SEQ ID NO. 134 |
| | | | Tyr (non- | | | |
| | | | receptor) | | | |
| 136 | ZAP70 | NP_001070.2 | Protein kinase, | Y598 | MRACYySLASK | SEQ ID NO. 135 |
| | | | Tyr (non- | | | |
| | | | receptor) | | | |
| 137 | TIE1 | NP_005415.1 | Protein kinase, | Y1083 | NCDDEVyELMR | SEQ ID NO. 136 |
| | | | Tyr (receptor) | | | |
| 138 | TrkC | NP_002521.2 | Protein kinase, | Y558 | VFLAECyNLSPTKDK | SEQ ID NO. 137 |
| | | | Tyr (receptor) | | | |
| 139 | TrkC | NP_002521.2 | Protein kinase, | Y786 | EVyDVMLGCWQR | SEQ ID NO. 138 |
| | | | Tyr (receptor) | | | |
| 140 | Tyro3 | NP_006284.2 | Protein kinase, | Y685 | KIYSGDyYRQGCASKLPVK | SEQ ID NO. 139 |
| | | | Tyr (receptor) | | | |
| 141 | Tyro3 | NP_006284.2 | Protein kinase, | Y686 | KIYSGDYyRQGCASKLPVK | SEQ ID NO. 140 |
| | | | Tyr (receptor) | | | |
| 142 | SLC20A2 | NP_006740.1 | Receptor, | Y423 | LVGDTVSySK | SEQ ID NO. 141 |
| | | | channel, | | | |
| | | | transporter or | | | |
| | | | cell surface | | | |
| | | | protein | | | |
| 143 | SLC20A2 | NP_006740.1 | Receptor, | Y646 | NIFVAWFVTVPVAGLFSAAVMALLMyGILPYV | SEQ ID NO. 142 |
| | | | channel, | | | |
| | | | transporter or | | | |
| | | | cell surface | | | |
| | | | protein | | | |
| 144 | SLC20A2 | NP_006740.1 | Receptor, | Y651 | NIFVAWFVTVPVAGLFSAAVMALLMYGILPyV | SEQ ID NO. 143 |
| | | | channel, | | | |
| | | | transporter or | | | |
| | | | cell surface | | | |
| | | | protein | | | |
| 145 | SLC25A12 | NP_003696.2 | Receptor, | Y369 | GSGSVVGELMyK | SEQ ID NO. 144 |
| | | | channel, | | | |
| | | | transporter or | | | |
| | | | cell surface | | | |
| | | | protein | | | |
| 146 | SLC38A2 | NP_061849.2 | Receptor, | Y30 | FSISPDEDSSSYSSNSDFNYSyPTK | SEQ ID NO. 145 |
| | | | channel, | | | |
| | | | transporter or | | | |
| | | | cell surface | | | |
| | | | protein | | | |
| 147 | SLC4A7 | NP_003606.3 | Receptor, | Y44 | AVyIGVHVPFSK | SEQ ID NO. 146 |
| | | | channel, | | | |
| | | | transporter or | | | |
| | | | cell surface | | | |
| | | | protein | | | |
| 148 | SNX6 | NP_067072.3 | Receptor, | Y137 | IGSSLyALGTQDSTDICK | SEQ ID NO. 147 |
| | | | channel, | | | |
| | | | transporter or | | | |
| | | | cell surface | | | |
| | | | protein | | | |
| 149 | SORL1 | NP_003096.1 | Receptor, | Y136 | SNVIVALARDSLALARPKSSDVyVSYDYGKSFK | SEQ ID NO. 148 |
| | | | channel, | | | |
| | | | transporter or | | | |
| | | | cell surface | | | |
| | | | protein | | | |
| 150 | SORL1 | NP_003096.1 | Receptor, | Y139 | SNVIVALARDSLALARPKSSDVYVSyDYGKSFK | SEQ ID NO. 149 |
| | | | channel, | | | |
| | | | transporter or | | | |
| | | | cell surface | | | |
| | | | protein | | | |
| 151 | TNF-R1 | NP_001056.1 | Receptor, | Y318 | REVAPPyQGADPILATALASDPIPNPLQK | SEQ ID NO. 150 |
| | | | channel, | | | |
| | | | transporter or | | | |
| | | | cell surface | | | |
| | | | protein | | | |
| 152 | TNF-R1 | NP_001056.1 | Receptor, | Y360 | WEDSAHKPQSLDTDDPATLyAVVENVPPLR | SEQ ID NO. 151 |
| | | | channel, | | | |
| | | | transporter or | | | |
| | | | cell surface | | | |
| | | | protein | | | |
| 153 | TRPM3 | NP_066003.2 | Receptor, | Y250 | INESLRDQLLVTIQKTFTy | SEQ ID NO. 152 |
| | | | channel, | | | |
| | | | transporter or | | | |
| | | | cell surface | | | |
| | | | protein | | | |
| 154 | TSPAN15 | NP_036471.1 | Receptor, | Y97 | ASLRDNLYLLQAFMyILGICL | SEQ ID NO. 153 |
| | | | channel, | | | |
| | | | transporter or | | | |
| | | | cell surface | | | |
| | | | protein | | | |
| 155 | XG | NP_780778.1 | Receptor, | Y52 | PPyYPQPENPDSGGNIYPRPKPR | SEQ ID NO. 154 |
| | | | channel, | | | |
| | | | transporter or | | | |
| | | | cell surface | | | |
| | | | protein | | | |
| 156 | snRNP116 | NP_004238.2 | RNA binding | Y167 | KRYDQDLCyTDILFTEQER | SEQ ID NO. 155 |
| | | | protein | | | |
| 157 | SNRPN | NP_003088.1 | RNA binding | Y15 | MLQHIDyR | SEQ ID NO. 156 |
| | | | protein | | | |
| 158 | SRm300 | NP_057417.3 | RNA binding | Y145 | AAFGISDSyVDGSSFDPQR | SEQ ID NO. 157 |
| | | | protein | | | |
| 159 | SRm300 | NP_057417.3 | RNA binding | Y1145 | FQSDSSSyPTVDSNSLLGQSR | SEQ ID NO. 158 |
| | | | protein | | | |
| 160 | SRm300 | NP_057417.3 | RNA binding | Y2390 | VPLSAyER | SEQ ID NO. 159 |
| | | | protein | | | |
| 161 | SRp46 | NP_115285.1 | RNA binding | Y175 | SPySRSRYRESRYGGSHYSSSGYSNSR | SEQ ID NO. 160 |
| | | | protein | | | |
| 162 | SRp46 | NP_115285.1 | RNA binding | Y180 | SPYSRSRyRESRYGGSHYSSSGYSNSR | SEQ ID NO. 161 |
| | | | protein | | | |
| 163 | SRp46 | NP_115285.1 | RNA binding | Y190 | SPYSRSRYRESRYGGSHySSSGYSNSR | SEQ ID NO. 162 |
| | | | protein | | | |
| 164 | STAU2 | NP_055208.1 | RNA binding | Y388 | GILHLSPDVyQEMEASR | SEQ ID NO. 163 |
| | | | protein | | | |
| 165 | TAF15 | NP_003478.1 | RNA binding | Y67 | SQSYGGyENQKQSSY | SEQ ID NO. 164 |
| | | | protein | | | |
| 166 | TAF15 | NP_003478.1 | RNA binding | Y491 | GGYGGDRGGGyGGDRGGYGGDRGGYGGDR | SEQ ID NO. 165 |
| | | | protein | | | |
| 167 | TARDBP | NP_031401.1 | RNA binding | Y155 | FTEyETQVK | SEQ ID NO. 166 |
| | | | protein | | | |
| 168 | U5-200kD | NP_054733.2 | RNA binding | Y1682 | IHAYVDYPIyDVLQMVGHANRPLQDDEGR | SEQ ID NO. 167 |
| | | | protein | | | |
| 169 | U5-200kD | NP_054733.2 | RNA binding | Y1770 | RMTQNPNyYNLQGISHR | SEQ ID NO. 168 |
| | | | protein | | | |
| 170 | UPF2 | NP_056357.1 | RNA binding | Y974 | KSLEVWTKDHPFPIDIDyMISDTLELLR | SEQ ID NO. 169 |
| | | | protein | | | |
| 171 | UPF3B | NP_075386.1 | RNA binding | Y160 | VGTIDDDPEyR | SEQ ID NO. 170 |
| | | | protein | | | |
| 172 | UPF3B | NP_075386.1 | RNA binding | Y429 | NKDRPAMQLyQPGAR | SEQ ID NO. 171 |
| | | | protein | | | |
| 173 | ZNF265 | NP_005446.2 | RNA binding | Y102 | TGyGGGFNER | SEQ ID NO. 172 |
| | | | protein | | | |
| 174 | ZNF265 | NP_005446.2 | RNA binding | Y114 | ENVEyIEREESDGEYDEFGRK | SEQ ID NO. 173 |
| | | | protein | | | |
| 175 | ZNF265 | NP_005446.2 | RNA binding | Y124 | ENVEYIEREESDGEyDEFGRK | SEQ ID NO. 174 |
| | | | protein | | | |
| 176 | SMARCE1 | NP_003070.3 | Transcriptional | Y142 | AYHNSPAYLAyINAK | SEQ ID NO. 175 |
| | | | regulator | | | |
| 177 | SMRT | NP_006303.3 | Transcriptional | Y2249 | SAVyPLLYR | SEQ ID NO. 176 |
| | | | regulator | | | |
| 178 | SND1 | NP_055205.2 | Transcriptional | Y113 | TPQGREYGMIyLGKDTNGENIAESLVAEGLATR | SEQ ID NO. 177 |
| | | | regulator | | | |
| 179 | SPT5 | NP_003160.2 | Transcriptional | Y140 | DQREEELGEyYMK | SEQ ID NO. 178 |
| | | | regulator | | | |
| 180 | SPT5 | NP_003160.2 | Transcriptional | Y295 | GIYKDDIAQVDyVEPSQNTISLK | SEQ ID NO. 179 |
| | | | regulator | | | |
| 181 | SPT5 | NP_003160.2 | Transcriptional | Y778 | TPMYGSQTPMyGSGSR | SEQ ID NO. 180 |
| | | | regulator | | | |
| 182 | SPT5 | NP_003160.2 | Transcriptional | Y787 | TPMyGSQTPLQDGSR | SEQ ID NO. 181 |
| | | | regulator | | | |
| 183 | SS18L1 | NP_945173.1 | Transcriptional | Y272 | yPDGHGDYAYQQSSYTEQSYDRSF | SEQ ID NO. 182 |
| | | | regulator | | | |
| 184 | SS18L1 | NP_945173.1 | Transcriptional | Y281 | YPDGHGDYAyQQSSYTEQSYDRSF | SEQ ID NO. 183 |
| | | | regulator | | | |
| 185 | SS18L1 | NP_945173.1 | Transcriptional | Y286 | YPDGHGDYAYQQSSyTEQSYDRSF | SEQ ID NO. 184 |
| | | | regulator | | | |
| 186 | SSB | NP_003133.1 | Transcriptional | Y23 | ICHQIEyYFGDFNLPR | SEQ ID NO. 185 |
| | | | regulator | | | |
| 187 | SSRP1 | NP_003137.1 | Transcriptional | Y452 | EGMNPSYDEYADSDEDQHDAyLER | SEQ ID NO. 186 |
| | | | regulator | | | |
| 188 | STAT1 | NP_009330.1 | Transcriptional | Y106 | RNLQDNFQEDPIQMSMIIySCLKEER | SEQ ID NO. 187 |
| | | | regulator | | | |
| 189 | STAT1 | NP_009330.1 | Transcriptional | Y668 | YLyPNIDKDHAFGK | SEQ ID NO. 188 |
| | | | regulator | | | |
| 190 | STAT3 | NP_644805.1 | Transcriptional | Y45 | QFLAPWIESQDWAyAASK | SEQ ID NO. 189 |
| | | | regulator | | | |
| 191 | STAT3 | NP_644805.1 | Transcriptional | Y674 | IMDATNILVSPLVYLyPDIPKEEAFGK | SEQ ID NO. 190 |
| | | | regulator | | | |
| 192 | STAT3 | NP_644805.1 | Transcriptional | Y686 | yCRPESQEHPEADPGSMPYLK | SEQ ID NO. 191 |
| | | | regulator | | | |
| 193 | STAT5B | NP_036580.2 | Transcriptional | Y171 | KLQQTQEyFIIQYQESLR | SEQ ID NO. 192 |
| | | | regulator | | | |
| 194 | STAT5B | NP_036580.2 | Transcriptional | Y683 | YyTPVPCESATAK | SEQ ID NO. 193 |
| | | | regulator | | | |
| 195 | TAF172 | NP_003963.1 | Transcriptional | Y415 | yALAVRQDVINTLLPK | SEQ ID NO. 194 |
| | | | regulator | | | |
| 196 | TAF6 | NP_005632.1 | Transcriptional | Y253 | RAEALQSIATDPGLyQMLPR | SEQ ID NO. 195 |
| | | | regulator | | | |
| 197 | TAT-SF1 | NP_055315.2 | Transcriptional | Y50 | DGDTQTDAGGEPDSLGQQPTDTPyEWDLDKK | SEQ ID NO. 196 |
| | | | regulator | | | |
| 198 | TAT-SF1 | NP_055315.2 | Transcriptional | Y634 | EFDEDSDEKEEEEDTyEK | SEQ ID NO. 197 |
| | | | regulator | | | |
| 199 | TAT-SF1 | NP_055315.2 | Transcriptional | Y650 | VFDDESDEKEDEEyADEK | SEQ ID NO. 198 |
| | | | regulator | | | |
| 200 | TCERG1 | NP_006697.2 | Transcriptional | Y149 | TPDGKVYyYNARTR | SEQ ID NO. 199 |
| | | | regulator | | | |
| 201 | TCERG1 | NP_006697.2 | Transcriptional | Y150 | TPDGKVYYyNARTR | SEQ ID NO. 200 |
| | | | regulator | | | |
| 202 | TCF12 | NP_003196.1 | Transcriptional | Y82 | GFTDSPHySDHLNDSR | SEQ ID NO. 201 |
| | | | regulator | | | |
| 203 | TCF12 | NP_003196.1 | Transcriptional | Y156 | QDLGLGSPAQLSSSGKPGTAyYSFSATSSR | SEQ ID NO. 202 |
| | | | regulator | | | |
| 204 | TCF12 | NP_003196.1 | Transcriptional | Y157 | QDLGLGSPAQLSSSGKPGTAYySFSATSSR | SEQ ID NO. 203 |
| | | | regulator | | | |
| 205 | TCF20 | NP_005641.1 | Transcriptional | Y839 | QINLTDyPIPR | SEQ ID NO. 204 |
| | | | regulator | | | |
| 206 | TEF-3 | NP_003204.2 | Transcriptional | Y349 | QVVEKVETEyARYENGHYSYRIHR | SEQ ID NO. 205 |
| | | | regulator | | | |
| 207 | TEF-3 | NP_003204.2 | Transcriptional | Y352 | QVVEKVETEYARyENGHYSYRIHR | SEQ ID NO. 206 |
| | | | regulator | | | |
| 208 | TEF-3 | NP_003204.2 | Transcriptional | Y357 | QVVEKVETEYARYENGHySYRIHR | SEQ ID NO. 207 |
| | | | regulator | | | |
| 209 | Tel | NP_001978.1 | Transcriptional | Y60 | LQPIyWSRDDVAQWLK | SEQ ID NO. 208 |
| | | | regulator | | | |
| 210 | Tel | NP_001978.1 | Transcriptional | Y114 | YRSPHSGDVLyELLQHILK | SEQ ID NO. 209 |
| | | | regulator | | | |
| 211 | Tel | NP_001978.1 | Transcriptional | Y346 | LLWDYVyQLLSDSR | SEQ ID NO. 210 |
| | | | regulator | | | |
| 212 | Tel | NP_001978.1 | Transcriptional | Y401 | HyYKLNIIR | SEQ ID NO. 211 |
| | | | regulator | | | |
| 213 | Tel | NP_001978.1 | Transcriptional | Y402 | HYyKLNIIR | SEQ ID NO. 212 |
| | | | regulator | | | |
| 214 | TFIIF | NP_004119.1 | Transcriptional | Y124 | AECRPAASENyMR | SEQ ID NO. 213 |
| | | | regulator | | | |
| 215 | TFII-I | NP_001509.2 | Transcriptional | Y419 | EDLQLDKPASGVKEEWyAR | SEQ ID NO. 214 |
| | | | regulator | | | |
| 216 | TORC2 | NP_859066.1 | Transcriptional | Y488 | LPPYPySSPSLVLPTQPHTPK | SEQ ID NO. 215 |
| | | | regulator | | | |
| 217 | TRAP150 | NP_005110.1 | Transcriptional | Y228 | ASESSKPWPDATyGTGSASR | SEQ ID NO. 216 |
| | | | regulator | | | |
| 218 | Trap170 | NP_004220.2 | Transcriptional | Y941 | DGAySLFDNSK | SEQ ID NO. 217 |
| | | | regulator | | | |
| 219 | TRIM22 | NP_006065.2 | Transcriptional | Y394 | SSGFAFDPSVNySK | SEQ ID NO. 218 |
| | | | regulator | | | |
| 220 | TRIP11 | NP_004230.1 | Transcriptional | Y452 | LNNEyEVIK | SEQ ID NO. 219 |
| | | | regulator | | | |
| 221 | TRIP11 | NP_004230.1 | Transcriptional | Y1267 | LQVDyTGLIQSYEQNETK | SEQ ID NO. 220 |
| | | | regulator | | | |
| 222 | TSC22D2 | NP_055594.1 | Transcriptional | Y703 | SHLMyAVR | SEQ ID NO. 221 |
| | | | regulator | | | |
| 223 | UXT | NP_004173.1 | Transcriptional | Y43 | DKVyEQLAK | SEQ ID NO. 222 |
| | | | regulator | | | |
| 224 | YY1 | NP_003394.1 | Transcriptional | Y251 | DIDHETVVEEQIIGENSPPDySEYMTGK | SEQ ID NO. 223 |
| | | | regulator | | | |
| 225 | YY1 | NP_003394.1 | Transcriptional | Y254 | DIDHETVVEEQIIGENSPPDYSEyMTGK | SEQ ID NO. 224 |
| | | | regulator | | | |
| 226 | ZBTB16 | NP_005997.2 | Transcriptional | Y334 | HLGIySVLPNHK | SEQ ID NO. 225 |
| | | | regulator | | | |
| 227 | ZNF141 | NP_003432.1 | Transcriptional | Y423 | KIHSADKPyKCK | SEQ ID NO. 226 |
| | | | regulator | | | |
| 228 | ZNF281 | NP_036614.1 | Transcriptional | Y353 | CDTCQQyFSR | SEQ ID NO. 227 |
| | | | regulator | | | |
| 229 | ZNF281 | NP_036614.1 | Transcriptional | Y479 | KNTDKNyLNFVSPLPDIVGQK | SEQ ID NO. 228 |
| | | | regulator | | | |
| 230 | ZNF289 | NP_115765.2 | Transcriptional | Y278 | LAyQELQIDRK | SEQ ID NO. 229 |
| | | | regulator | | | |
| 231 | ZNF33A | NP_008885.1 | Transcriptional | Y45 | DVMLENySNLVSVGYCVHK | SEQ ID NO. 230 |
| | | | regulator | | | |
| 232 | TSFM | NP_005717.2 | Translational | Y59 | TGySFVNCK | SEQ ID NO. 231 |
| | | | regulator | | | |
| 233 | TUFM | NP_003312.3 | Translational | Y269 | DLEKPFLLPVEAVySVPGR | SEQ ID NO. 232 |
| | | | regulator | | | |
| 234 | UBC3B | NP_060281.2 | Ubiquitin | Y190 | VPTTLAEyCIK | SEQ ID NO. 233 |
| | | | conjugating | | | |
| | | | system | | | |
| 235 | UBCE7IP3 | NP_006453.1 | Ubiquitin | Y220 | QQQQEGNyLQHVQLDQR | SEQ ID NO. 234 |
| | | | conjugating | | | |
| | | | system | | | |
| 236 | UBE1L | NP_003326.2 | Ubiquitin | Y15 | LLDEELySR | SEQ ID NO. 235 |
| | | | conjugating | | | |
| | | | system | | | |
| 237 | ubiquilin2 | NP_038472.2 | Ubiquitin | Y265 | ALSNLESIPGGyNALR | SEQ ID NO. 236 |
| | | | conjugating | | | |
| | | | system | | | |
| 238 | ubiquitin | NP_002945.1 | Ubiquitin | Y148 | CCLTyCFNKPEDK | SEQ ID NO. 237 |
| | | | conjugating | | | |
| | | | system | | | |
| 239 | UREB1 | NP_113584.3 | Ubiquitin | Y1658 | YTVQFTTMVQVNEETGNR | SEQ ID NO. 238 |
| | | | conjugating | | | |
| | | | system | | | |
| 240 | UREB1 | NP_113584.3 | Ubiquitin | Y3424 | SVPVSAGGEGETSPySLEASPLGQLMNMLSHPVI | SEQ ID NO. 239 |
| | | | conjugating | | RR | |
| | | | system | | | |
| 241 | UREB1 | NP_113584.3 | Ubiquitin | Y4078 | EMFNPMyALFR | SEQ ID NO. 240 |
| | | | conjugating | | | |
| | | | system | | | |
| 242 | USP10 | NP_005144.2 | Ubiquitin | Y54 | SSVELPPYSGTVLCGTQAVDKLPDGQEyQR | SEQ ID NO. 241 |
| | | | conjugating | | | |
| | | | system | | | |
| 243 | USP11 | NP_004642.2 | Ubiquitin | Y396 | SQFLGyQQHDSQE | SEQ ID NO. 242 |
| | | | conjugating | | | |
| | | | system | | | |
| 244 | USP14 | NP_005142.1 | Ubiquitin | Y417 | YEPFSFADDIGSNNCGyYDLQAVLTHQGR | SEQ ID NO. 243 |
| | | | conjugating | | | |
| | | | system | | | |
| 245 | USP15 | NP_006304.1 | Ubiquitin | Y239 | NSNYCLPSyTAYK | SEQ ID NO. 244 |
| | | | conjugating | | | |
| | | | system | | | |
| 246 | USP20 | NP_006667.2 | Ubiquitin | Y227 | GyAQQDTQEFLR | SEQ ID NO. 245 |
| | | | conjugating | | | |
| | | | system | | | |
| 247 | USP4 | NP_003354.2 | Ubiquitin | Y192 | YMSNTyEQLSK | SEQ ID NO. 246 |
| | | | conjugating | | | |
| | | | system | | | |
| 248 | USP4 | NP_003354.2 | Ubiquitin | Y476 | VSVTFDPFCyLTLPLPLKK | SEQ ID NO. 247 |
| | | | conjugating | | | |
| | | | system | | | |
| 249 | WWP2 | NP_008945.2 | Ubiquitin | Y369 | NyEQWQSQR | SEQ ID NO. 248 |
| | | | conjugating | | | |
| | | | system | | | |
| 250 | SLFN5 | XP_496206.1 | Unknown function | Y115 | AEVENKGySYK | SEQ ID NO. 249 |
| 251 | SLITRK5 | NP_056382.1 | Unknown function | Y833 | SPAySVSTIEPR | SEQ ID NO. 250 |
| 252 | SMAP-5 | NP_110426.4 | Unknown function | Y26 | SIDDQSQQSyDYGGSGGPY | SEQ ID NO. 251 |
| 253 | SMAP-5 | NP_110426.4 | Unknown function | Y28 | SIDDQSQQSYDyGGSGGPY | SEQ ID NO. 252 |
| 254 | SMAP-5 | NP_110426.4 | Unknown function | Y67 | TGQIyQPTQAY | SEQ ID NO. 253 |
| 255 | SMBP | NP_064508.2 | Unknown function | Y272 | YSKEEEMDDMDRDLGDEyGWK | SEQ ID NO. 254 |
| 256 | SMC6L1 | NP_078900.1 | Unknown function | Y780 | SLKIEAENKyDAIK | SEQ ID NO. 255 |
| 257 | SMEK2 | NP_065196.1 | Unknown function | Y171 | EKLALALENEGyIK | SEQ ID NO. 256 |
| 258 | SNF8 | NP_009172.2 | Unknown function | Y192 | NGyVTVSEIK | SEQ ID NO. 257 |
| 259 | SNX14 | NP_065201.1 | Unknown function | Y353 | SLHEELQKIyKTY | SEQ ID NO. 258 |
| 260 | SPATA2 | NP_006029.1 | Unknown function | Y257 | SVDAyDSYWESR | SEQ ID NO. 259 |
| 261 | SPATA2 | NP_006029.1 | Unknown function | Y260 | SVDAYDSyWESR | SEQ ID NO. 260 |
| 262 | SPATA5 | NP_660208.1 | Unknown function | Y180 | IVLPGNFLyCTFYGRPYK | SEQ ID NO. 261 |
| 263 | SPATA5 | NP_660208.1 | Unknown function | Y793 | IIyVPLPDAATRR | SEQ ID NO. 262 |
| 254 | SSX9 | NP_777622.1 | Unknown function | Y48 | SSEKIIyVYMKR | SEQ ID NO. 263 |
| 265 | SSX9 | NP_777622.1 | Unknown function | Y50 | SSEKIIYVyMKR | SEQ ID NO. 264 |
| 266 | STS-1 | NP_116262.2 | Unknown function | Y402 | MDVVFGKyWLSQCFDAK | SEQ ID NO. 265 |
| 267 | SYAP1 | NP_116185.2 | Unknown function | Y60 | DFGNyLFNFASAATK | SEQ ID NO. 266 |
| 268 | SYAP1 | NP_116185.2 | Unknown function | Y327 | ELQQELQEyEVVTESEKR | SEQ ID NO. 267 |
| 269 | SYNPO | NP_009217.3 | Unknown function | Y222 | MSGRAAATTPTKVySE | SEQ ID NO. 268 |
| 270 | TBC1D12 | XP_051081.4 | Unknown function | Y800 | LyEDILLQMDFIHIAQFLTK | SEQ ID NO. 269 |
| 271 | TBC1D13 | NP_060671.2 | Unknown function | Y143 | ATDyPCLLILDPQNEFETLR | SEQ ID NO. 270 |
| 272 | TDRD3 | NP_110421.1 | Unknown function | Y481 | HFNVNTDyQNPVR | SEQ ID NO. 271 |
| 273 | TDRD3 | NP_110421.1 | Unknown function | Y644 | STRPTQQFyQPPR | SEQ ID NO. 272 |
| 274 | TIP20 | NP_001002836.1 | Unknown function | Y150 | IHTGEKPyTCPDCGR | SEQ ID NO. 273 |
| 275 | TIPRL | NP_690866.1 | Unknown function | Y209 | LYHEADKTyMLR | SEQ ID NO. 274 |
| 276 | TLCD1 | NP_612472.1 | Unknown function | Y173 | VNKyVNLVMYFLFR | SEQ ID NO. 275 |
| 277 | TLCD1 | NP_612472.1 | Unknown function | Y179 | VNKYVNLVMyFLFR | SEQ ID NO. 276 |
| 278 | TNKS1BP1 | NP_203754.2 | Unknown function | Y855 | DSQGTySSRDAELQDQEFGKR | SEQ ID NO. 277 |
| 279 | TNKS1BP1 | NP_203754.2 | Unknown function | Y940 | DVSLGTyGSR | SEQ ID NO. 278 |
| 280 | TNKS1BP1 | NP_203754.2 | Unknown function | Y1122 | SyQFGIIGNDR | SEQ ID NO. 279 |
| 281 | TNRC6B | NP_055903.1 | Unknown function | Y1081 | DMGTTDSGPyFEKGGSHGLFGNSTAQSR | SEQ ID NO. 280 |
| 282 | TNRC6B | NP_055903.1 | Unknown function | Y1388 | GIQNIDPESDPyVTPGSVLGGTATSPIVDTDHQL | SEQ ID NO. 281 |
| | | | | | LR | |
| 283 | TOM1 | NP_005479.1 | Unknown function | Y386 | yEAPQATDGLAGALDAR | SEQ ID NO. 282 |
| 284 | TPD52 | NP_005070.1 | Unknown function | Y96 | GWQDVTATSAyK | SEQ ID NO. 283 |
| 285 | TPD52L2 | NP_003279.2 | Unknown function | Y106 | SWHDVQVSSAyVK | SEQ ID NO. 284 |
| 286 | TPR18 | NP_660153.2 | Unknown function | Y1023 | EYEKAKKTyMQACK | SEQ ID NO. 285 |
| 287 | TRIM16 | NP_006461.3 | Unknown function | Y371 | EQFLQyAYDITFDPDTAHKYLRLQEENRK | SEQ ID NO. 286 |
| 288 | TRIM34 | NP_067629.2 | Unknown function | Y391 | yRPLFGYWVIGLQNKCK | SEQ ID NO. 287 |
| 289 | TRIM34 | NP_067629.2 | Unknown function | Y397 | YRPLFGyWVIGLQNKCK | SEQ ID NO. 288 |
| 290 | TRIM9 | NP_443210.1 | Unknown function | Y524 | TGVSPySKTLVLQTSEGK | SEQ ID NO. 289 |
| | iso5 | | | | | |
| 291 | TRS85 | NP_055754.2 | Unknown function | Y179 | IQHNSDySYPK | SEQ ID NO. 290 |
| 292 | TRS85 | NP_055754.2 | Unknown function | Y181 | IQHNSDYSyPK | SEQ ID NO. 291 |
| 293 | TSC22D2 | NP_055594.1 | Unknown function | Y57 | ATDyGPEEVCER | SEQ ID NO. 292 |
| 294 | TTC21B | NP_079029.3 | Unknown function | Y160 | GKEPyTKKALK | SEQ ID NO. 293 |
| 295 | UBAP2 | NP_060919.2 | Unknown function | Y199 | FSTQGMGTFNPADySDSTSTDVCGTK | SEQ ID NO. 294 |
| 296 | UBAP2 | NP_060919.2 | Unknown function | Y1111 | SQASKPAyGNSPYWTN | SEQ ID NO. 295 |
| 297 | UBAP2 | NP_060919.2 | Unknown function | Y1116 | SQASKPAYGNSPyWTN | SEQ ID NO. 296 |
| 298 | USH3A | NP_443721.1 | Unknown function | Y95 | HLSEKIANyKEGTY | SEQ ID NO. 297 |
| 299 | USP47 | BAB55063.1 | Unknown function | Y81 | EGSVGSTSDyVSQSYSYSSILNK | SEQ ID NO. 298 |
| 300 | USP47 | BAB55063.1 | Unknown function | Y86 | EGSVGSTSDYVSQSySYSSILNK | SEQ ID NO. 299 |
| 301 | UTX | NP_066963.1 | Unknown function | Y928 | PPSSPyPPLPKDKLNPPTPSIYLENK | SEQ ID NO. 300 |
| 302 | VPS13B | NP_060360.3 | Unknown function | Y242 | LHFTyENLNSK | SEQ ID NO. 301 |
| 303 | VPS13B | NP_060360.3 | Unknown function | Y2100 | KIKNAHSLAHSEETSAMSNTMVNKDDLPVSKyYR | SEQ ID NO. 302 |
| 304 | VPS13B | NP_060360.3 | Unknown function | Y2101 | KIKNAHSLAHSEETSAMSNTMVNKDDLPVSKYyR | SEQ ID NO. 303 |
| 305 | VPS13D | NP_056193.2 | Unknown function | Y661 | VHTSGFGyQSELELR | SEQ ID NO. 304 |
| 306 | WAPL | NP_055860.1 | Unknown function | Y633 | REDKELyTVVQHVK | SEQ ID NO. 305 |
| 307 | WBP2 | NP_036610.2 | Unknown function | Y231 | AAEAAASAyYNPGNPHNVYMPTSQPPPPPYYPPE | SEQ ID NO. 306 |
| | | | | | DK | |
| 308 | WBSCR20A | NP_060514.1 | Unknown function | Y37 | QLyALVCETQR | SEQ ID NO. 307 |
| 309 | WDR18 | NP_077005.2 | Unknown function | Y61 | NyISAWELQR | SEQ ID NO. 308 |
| 310 | WDR3 | NP_006775.1 | Unknown function | Y456 | TMTCEyALCSFFVPGDR | SEQ ID NO. 309 |
| 311 | WDR43 | BAA13441.1 | Unknown function | Y196 | MSLLLVyGSWFQPTIER | SEQ ID NO. 310 |
| 312 | WDR70 | NP_060504.1 | Unknown function | Y618 | AAEDSPyWVSPAYSK | SEQ ID NO. 311 |
| 313 | WDR9 | NP_061836.2 | Unknown function | Y551 | PyEKIPDQMFFHTDYR | SEQ ID NO. 312 |
| 314 | WHSC1 | NP_579877.1 | Unknown function | Y747 | KyPLTVFESRGFR | SEQ ID NO. 313 |
| 315 | YEATS2 | NP_060493.3 | Unknown function | Y15 | TIKETDPDyEDVSVALPNKR | SEQ ID NO. 314 |
| 316 | YRDC | NP_078916.3 | Unknown function | Y278 | YGLLPSHASyL | SEQ ID NO. 315 |
| 317 | Za2Od3 | NP_061879.2 | Unknown function | Y185 | YSDVHNCSYNyK | SEQ ID NO. 316 |
| 318 | ZBTB11 | NP_055230.1 | Unknown function | Y517 | SVNEGAyIR | SEQ ID NO. 317 |
| 319 | ZBTB5 | NP_055687.1 | Unknown function | Y641 | VHTGEKPyACLK | SEQ ID NO. 318 |
| 320 | ZC3H7A | NP_054872.2 | Unknown function | Y681 | RyWQNLEANVPGAQVLGNQIMPGFLNMKIK | SEQ ID NO. 319 |
| 321 | ZDHHC11 | NP_079062.1 | Unknown function | Y106 | LMKNySQPMPLFDRSK | SEQ ID NO. 320 |
| 322 | ZDHHC13 | NP_061901.2 | Unknown function | Y59 | ATQyGIFER | SEQ ID NO. 321 |
| 323 | ZDHHC5 | NP_056272.2 | Unknown function | Y354 | yRPGYSSSSTSAAMPHSSSAK | SEQ ID NO. 322 |
| 324 | ZDHHC5 | NP_056272.2 | Unknown function | Y358 | YRPGySSSSTSAAMPHSSSAK | SEQ ID NO. 323 |
| 325 | ZDHHC5 | NP_056272.2 | Unknown function | Y630 | GVGSPEPGPTAPyLGR | SEQ ID NO. 324 |
| 326 | ZDHHC8 | NP_037505.1 | Unknown function | Y579 | SQADSLFGDSGVyDAPSSYSLQQASVLSEGPRGP | SEQ ID NO. 325 |
| | | | | | ALR | |
| 327 | ZFP 598 | NP_835461.1 | Unknown function | Y108 | KYDIYFADGKVyALYR | SEQ ID NO. 326 |
| 328 | ZFP 598 | NP_835461.1 | Unknown function | Y289 | HIDLQFSyAPR | SEQ ID NO. 327 |
| 329 | ZFP 598 | NP_835461.1 | Unknown function | Y312 | RNEGVVGGEDYEEVDRySR | SEQ ID NO. 328 |
| 330 | ZFP 598 | NP_835461.1 | Unknown function | Y471 | LKDEDFPSLSASTSSSCSTAATPGPVGLALPyAI | SEQ ID NO. 329 |
| | | | | | PAR | |
| 331 | ZFPL1 | NP_006773.2 | Unknown function | Y235 | DDDRTPGLHGDCDDDKyR | SEQ ID NO. 330 |
| 332 | ZFR | NP_057191.2 | Unknown function | Y610 | RHRLQyKKKVNPDLQVEVK | SEQ ID NO. 331 |
| 333 | ZFR | NP_057191.2 | Unknown function | Y667 | RYEEDMyWR | SEQ ID NO. 332 |
| 334 | ZNF12 | NP_008887.2 | Unknown function | Y315 | THSGEKPyECSYCGK | SEQ ID NO. 333 |
| 335 | ZNF183 | NP_008909.1 | Unknown function | Y120 | SAKPVGPEDMGATAVyELDTEKER | SEQ ID NO. 334 |
| 336 | ZNF183 | NP_008909.1 | Unknown function | Y153 | GKEDDKIyR | SEQ ID NO. 335 |
| 337 | ZNF198 | NP_003444.1 | Unknown function | Y502 | FCCQSCVSEyK | SEQ ID NO. 336 |
| 338 | ZNF198 | NP_003444.1 | Unknown function | Y557 | FFDMTQCIGPNGyMEPYCSTACMNSHK | SEQ ID NO. 337 |
| 339 | ZNF198 | NP_003444.1 | Unknown function | Y595 | NSLPQyQATMPDGKLYNFCNSSCVAK | SEQ ID NO. 338 |
| 340 | ZNF198 | NP_003444.1 | Unknown function | Y605 | NSLPQYQATMPDGKLyNFCNSSCVAK | SEQ ID NO. 339 |
| 341 | ZNF198 | NP_003444.1 | Unknown function | Y671 | TCSDDyKKLHCIVTYCEYCQEEK | SEQ ID NO. 340 |
| 342 | ZNF198 | NP_003444.1 | Unknown function | Y680 | TCSDDYKKLHCIVTyCEYCQEEK | SEQ ID NO. 341 |
| 343 | ZNF198 | NP_003444.1 | Unknown function | Y683 | LHCIVTYCEyCQEEK | SEQ ID NO. 342 |
| 344 | ZNF198 | NP_003444.1 | Unknown function | Y729 | CVTCNyCSQLCK | SEQ ID NO. 343 |
| 345 | ZNF198 | NP_003444.1 | Unknown function | Y762 | KFQDWyYK | SEQ ID NO. 344 |
| 346 | ZNF198 | NP_003444.1 | Unknown function | Y819 | GPENLHyDQGCQTSR | SEQ ID NO. 345 |
| 347 | ZNF198 | NP_003444.1 | Unknown function | Y862 | ATyCKPHMQTK | SEQ ID NO. 346 |
| 348 | ZNF272 | NP_006626.3 | Unknown function | Y420 | HFNIHTGEKPyECLQCGK | SEQ ID NO. 347 |
| 349 | ZNF291 | NP_065894.1 | Unknown function | Y1207 | ENyTQNTIQVAIQSLR | SEQ ID NO. 348 |
| 350 | ZNF313 | NP_061153.1 | Unknown function | Y116 | YQNyIMEGVK | SEQ ID NO. 349 |
| 351 | ZNF313 | NP_061153.1 | Unknown function | Y186 | SVVCPICASMPWGDPNyR | SEQ ID NO. 350 |
| 352 | ZNF326 | NP_892021.1 | Unknown function | Y93 | SGyGFNEPEQSR | SEQ ID NO. 351 |
| 353 | ZNF330 | NP_055302.1 | Unknown function | Y262 | NLSSDKyGDTSYHDEEEDEYEAEDDEEEEDEGR | SEQ ID NO. 352 |
| 354 | ZNF330 | NP_055302.1 | Unknown function | Y267 | YGDTSyHDEEEDEYEAEDDEEEEDEGR | SEQ ID NO. 353 |
| 355 | ZNF330 | NP_055302.1 | Unknown function | Y275 | YGDTSYHDEEEDEyEAEDDEEEEDEGR | SEQ ID NO. 354 |
| 356 | ZNF330 | NP_055302.1 | Unknown function | Y312 | TYASGyAHYEEQEN | SEQ ID NO. 355 |
| 357 | ZNF341 | NP_116208.3 | Unknown function | Y174 | GPPPVQSSLNMHSVPSyLT | SEQ ID NO. 356 |
| 358 | ZNF425 | NP_001001661.1 | Unknown function | Y675 | PFQCPECDKSyCIR | SEQ ID NO. 357 |
| 359 | ZNF433 | NP_001073880.1 | Unknown function | Y132 | AyEYQEYGQKPYK | SEQ ID NO. 358 |
| 360 | ZNF433 | NP_001073880.1 | Unknown function | Y134 | AYEyQEYGQKPYK | SEQ ID NO. 359 |
| 361 | ZNF433 | NP_001073880.1 | Unknown function | Y137 | AYEYQEyGQKPYK | SEQ ID NO. 360 |
| 362 | ZNF433 | NP_001073880.1 | Unknown function | Y142 | AYEYQEYGQKPyK | SEQ ID NO. 361 |
| 363 | ZNF512 | NP_115810.2 | Unknown function | Y417 | IQCPNQGCEAVySSVSGLK | SEQ ID NO. 362 |
| 364 | ZNF532 | NP_060651.2 | Unknown function | Y811 | SPyTCPECGAICR | SEQ ID NO. 363 |
| 365 | ZNF563 | NP_660319.1 | Unknown function | Y237 | QCSKAFPFySSYR | SEQ ID NO. 364 |
| 366 | ZNF563 | NP_660319.1 | Unknown function | Y240 | QCSKAFPFYSSyR | SEQ ID NO. 365 |
| 367 | ZNF571 | NP_057620.3 | Unknown function | Y335 | VHTGEKPyICKECGK | SEQ ID NO. 366 |
| 368 | ZNF574 | NP_073589.3 | Unknown function | Y605 | YHHTGEyPYK | SEQ ID NO. 367 |
| 369 | ZNF577 | NP_116068.1 | Unknown function | Y41 | VLyKEVMLENYINLVSIGYRGTKPDSLFK | SEQ ID NO. 368 |
| 370 | ZNF592 | NP_055445.2 | Unknown function | Y768 | SPyCCPECGVLCR | SEQ ID NO. 369 |
| 371 | ZNF609 | NP_055857.1 | Unknown function | Y796 | AEADKIySFTDNAPSPSIGGSSR | SEQ ID NO. 370 |
| 372 | ZNF622 | NP_219482.1 | Unknown function | Y284 | DHSFFIPDIEyLSDIK | SEQ ID NO. 371 |
| 373 | ZSWIM4 | NP_075560.1 | Unknown function | Y548 | yLFTALLPHDPDLAYRLALR | SEQ ID NO. 372 |
| 374 | SNAP29 | NP_004773.1 | Vesicle protein | Y160 | yQASHPNLR | SEQ ID NO. 373 |
| 375 | SNX1 | NP_003090.2 | Vesicle protein | Y162 | IGDGMNAyVAYK | SEQ ID NO. 374 |
| 376 | SNX2 | NP_003091.2 | Vesicle protein | Y203 | YLHVGyIVPPAPEK | SEQ ID NO. 375 |
| 377 | SNX27 | NP_112180.4 | Vesicle protein | Y186 | FVVYNVyMAGR | SEQ ID NO. 376 |
| 378 | STX16 | NP_003754.2 | Vesicle protein | Y66 | WVDGVDEIQyDVGR | SEQ ID NO. 377 |
| 379 | STXBP5 | NP_640337.2 | Vesicle protein | Y502 | NKDDRPNTDIVDEDPyAIQIISWCPESR | SEQ ID NO. 378 |
| 380 | SV2A | NP_055664.2 | Vesicle protein | Y65 | FEEEDDDDDFPAPSDGyYR | SEQ ID NO. 379 |
| 381 | SV2A | NP_055664.2 | Vesicle protein | Y66 | FEEEDDDDDFPAPSDGYyR | SEQ ID NO. 380 |
| 382 | VPS29 | NP_057310.1 | Vesicle protein | Y69 | GDFDENLNyPEQK | SEQ ID NO. 381 |
| 383 | VPS35 | NP_060676.2 | Vesicle protein | Y507 | SEDPDQQyLILNTAR | SEQ ID NO. 382 |
| 384 | VPS4B | NP_004860.2 | Vesicle protein | Y40 | AGNYEEALQLYQHAVQyFLHVVK | SEQ ID NO. 383 |

The short name for each protein in which a phosphorylation site has presently been identified is provided in Column A, and its SwissProt accession number (human) is provided Column B. The protein type/group into which each protein falls is provided in Column C. The identified tyrosine residue at which phosphorylation occurs in a given protein is identified in Column D, and the amino acid sequence of the phosphorylation site encompassing the tyrosine residue is provided in Column E (lower case y=the tyrosine (identified in Column D)) at which phosphorylation occurs. Table 1 above is identical to FIG. 2, except that the latter includes the disease and cell type(s) in which the particular phosphorylation site was identified (Columns F and G).

“Antibody” or “antibodies” refers to all classes of immunoglobulins, including IgG, IgM, IgA, IgD, and IgE, including whole antibodies and any antigen biding fragment thereof (e.g., F_{ab}) or single chains thereof, including chimeric, polyclonal, and monoclonal antibodies. Antibodies are antigen-specific protein molecules produced by lymphocytes of the B cell lineage. Following antigenic stimulation, B cells that have surface immunoglobulin receptors that bind the antigen clonally expand, and the binding affinity for the antigen increases through a process called affinity maturation. The B cells further differentiate into plasma cells, which secrete large quantities of antibodies in to the serum. While the physiological role of antibodies is to protect the host animal by specifically binding and eliminating microbes and microbial pathogens from the body, large amounts of antibodies are also induced by intentional immunization to produce specific antibodies that are used extensively in many biomedical and therapeutic applications.

Antibody molecules are shaped somewhat like the letter “Y”, and consist of 4 protein chains, two heavy (H) and two light (L) chains. Antibodies possess two distinct and spatially separate functional features. The ends of each of the two arms of the “Y” contain the variable regions (variable heavy (V(H)) and variable light (V(L)) regions), which form two identical antigen-binding sites. The variable regions undergo a process of “affinity maturation” during the immune response, leading to a rapid divergence of amino acids within these variable regions. The other end of the antibody molecule, the stem of the “Y”, contains only the two heavy constant (CH) regions, interacts with effector cells to determine the effector functions of the antibody. There are five different CH region genes that encode the five different classes of immunoglobulins: IgM, IgD, IgG, IgA and IgE. These constant regions, by interacting with different effector cells and molecules, determine the immunoglobulin molecule's biological function and biological response.

Each V(H) and V(L) region contains three subregions called complementarity determining regions. These include CDR1-3 of the V(H) domain and CDR1-3 of the V(L) domain. These six CDRs generally form the antigen binding surface, and include those residues that hypermutate during the affinity maturation phase of the immune response. The CDR3 of the V(H) domain seems to play a dominant role in generating diversity oof both the B cell antigen receptor (BCR) and the T cell antigen receptor systems (Xu et al., Immunity 13:37-45 (2000)).

The term “antibody” or “antibodies” refers to all classes of polyclonal or monoclonal immunoglobulins, including IgG, IgM, IgA, IgD, and IgE, including whole antibodies and any antigen binding fragment thereof. This includes any combination of immunoglobulin domains or chains that contains a variable region (V(H) or V(L)) that retains the ability to bind the immunogen. Such fragments include F(ab)₂ fragments (V(H)-C(H1), V(L)-C(L))₂; monovalent Fab fragments (V(H)-C(H1), V(L)-C(L)); Fv fragment (V(H)-V(L); single-chain Fv fragments (Kobayashi et al., Steroids July; 67(8):733-42 (2002).

Monoclonal antibodies refer to clonal antibodies produced from fusions between immunized murine, rabbit, human, or other vertebrate species, and produced by classical fusion technology Kohler G, Milstein C. Continuous cultures of fused cells secreting antibody of predefined specificity. Nature 1975 Aug. 7; 256(5517):495-7 or by alternative methods which may isolate clones of immunoglobulin secreting cells from transformed plasma cells.

When used with respect to an antibody's binding to one phospho-form of a sequence, the expression “does not bind” means that a phospho-specific antibody either does not apparently bind to the non-phospho form of the antigen as ascertained in commonly used experimental detection systems (Western blotting, IHC, Immunofluorescence, etc.). One of skill in the art will appreciate that the expression may be applicable in those instances when (1) a phospho-specific antibody either does not apparently bind to the non-phospho form of the antigen as ascertained in commonly used experimental detection systems (Western blotting, IHC, Immunofluorescence, etc.); (2) where there is some reactivity with the surrounding amino acid sequence, but that the phosphorylated residue is an immunodominant feature of the reaction. In cases such as these, there is an apparent difference in affinities for the two sequences. Dilutional analyses of such antibodies indicates that the antibodies apparent affinity for the phosphorylated form is at least 10-100 fold higher than for the non-phosphorylated form; or where (3) the phospho-specific antibody reacts no more than an appropriate control antibody would react under identical experimental conditions. A control antibody preparation might be, for instance, purified immunoglobulin from a pre-immune animal of the same species, an isotype- and species-matched monoclonal antibody. Tests using control antibodies to demonstrate specificity are recognized by one of skill in the art as appropriate and definitive.

In the specification and the appended claims, the singular forms include plural referents unless the context clearly dictates otherwise. As used in this specification, the singular forms “a,” “an” and “the” specifically also encompass the plural forms of the terms to which they refer, unless the content clearly dictates otherwise. As used herein, unless specifically indicated otherwise, the word “or” is used in the “inclusive” sense of “and/or” and not the “exclusive” sense of “either/or.”

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable that is inherently discrete, the variable can be equal to any integer value of the numerical range, including the end-points of the range. Similarly, for a variable that is inherently continuous, the variable can be equal to any real value of the numerical range, including the end-points of the range. As an example, a variable that is described as having values between 0 and 2, can be 0, 1 or 2 for variables which are inherently discrete, and can be 0.0, 0.1, 0.01, 0.001, or any other real value for variables which are inherently continuous.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

“Antibody” or “antibodies” refers to all classes of immunoglobulins, including IgG, IgM, IgA, IgD, and IgE, including whole antibodies and any antigen biding fragment thereof (e.g., F_{ab}) or single chains thereof, including chimeric, polyclonal, and monoclonal antibodies. Antibodies are antigen-specific protein molecules produced by lymphocytes of the B cell lineage. Following antigenic stimulation, B cells that have surface immunoglobulin receptors that bind the antigen clonally expand, and the binding affinity for the antigen increases through a process called affinity maturation. The B cells further differentiate into plasma cells, which secrete large quantities of antibodies in to the serum. While the physiological role of antibodies is to protect the host animal by specifically binding and eliminating microbes and microbial pathogens from the body, large amounts of antibodies are also induced by intentional immunization to produce specific antibodies that are used extensively in many biomedical and therapeutic applications.

Antibody molecules are shaped somewhat like the letter “Y”, and consist of 4 protein chains, two heavy (H) and two light (L) chains. Antibodies possess two distinct and spatially separate functional features. The ends of each of the two arms of the “Y” contain the variable regions (variable heavy (V(H)) and variable light (V(L)) regions), which form two identical antigen-binding sites. The variable regions undergo a process of “affinity maturation” during the immune response, leading to a rapid divergence of amino acids within these variable regions. The other end of the antibody molecule, the stem of the “Y”, contains only the two heavy constant (CH) regions, interacts with effector cells to determine the effector functions of the antibody. There are five different CH region genes that encode the five different classes of immunoglobulins: IgM, IgD, IgG, IgA and IgE. These constant regions, by interacting with different effector cells and molecules, determine the immunoglobulin molecule's biological function and biological response.

Each V(H) and V(L) region contains three subregions called complementarity determining regions. These include CDR1-3 of the V(H) domain and CDR1-3 of the V(L) domain. These six CDRs generally form the antigen binding surface, and include those residues that hypermutate during the affinity maturation phase of the immune response. The CDR3 of the V(H) domain seems to play a dominant role in generating diversity oof both the B cell antigen receptor (BCR) and the T cell antigen receptor systems (Xu et al., Immunity 13:37-45 (2000)).

The term “antibody” or “antibodies” refers to all classes of polyclonal or monoclonal immunoglobulins, including IgG, IgM, IgA, IgD, and IgE, including whole antibodies and any antigen binding fragment thereof. This includes any combination of immunoglobulin domains or chains that contains a variable region (V(H) or V(L)) that retains the ability to bind the immunogen. Such fragments include F(ab)₂ fragments (V(H)-C(H1), V(L)-C(L))₂; monovalent Fab fragments (V(H)-C(H1), V(L)-C(L)); Fv fragment (V(H)-V(L); single-chain Fv fragments (Kobayashi et al., Steroids July; 67(8):733-42 (2002).

Monoclonal antibodies refer to clonal antibodies produced from fusions between immunized murine, rabbit, human, or other vertebrate species, and produced by classical fusion technology Kohler G, Milstein C. Continuous cultures of fused cells secreting antibody of predefined specificity. Nature 1975 Aug. 7; 256(5517):495-7 or by alternative methods which may isolate clones of immunoglobulin secreting cells from transformed plasma cells.

When used with respect to an antibody's binding to one phospho-form of a sequence, the expression “does not bind” means that a phospho-specific antibody either does not apparently bind to the non-phospho form of the antigen as ascertained in commonly used experimental detection systems (Western blotting, IHC, Immunofluorescence, etc.). One of skill in the art will appreciate that the expression may be applicable in those instances when (1) a phospho-specific antibody either does not apparently bind to the non-phospho form of the antigen as ascertained in commonly used experimental detection systems (Western blotting, IHC, Immunofluorescence, etc.); (2) where there is some reactivity with the surrounding amino acid sequence, but that the phosphorylated residue is an immunodominant feature of the reaction. In cases such as these, there is an apparent difference in affinities for the two sequences. Dilutional analyses of such antibodies indicates that the antibodies apparent affinity for the phosphorylated form is at least 10-100 fold higher than for the non-phosphorylated form; or where (3) the phospho-specific antibody reacts no more than an appropriate control antibody would react under identical experimental conditions. A control antibody preparation might be, for instance, purified immunoglobulin from a pre-immune animal of the same species, an isotype- and species-matched monoclonal antibody. Tests using control antibodies to demonstrate specificity are recognized by one of skill in the art as appropriate and definitive.

“Target signaling protein/polypeptide” means any protein (or polypeptide derived therefrom) enumerated in Column A of Table 1/FIG. 2, which is disclosed herein as being phosphorylated in one or more cell line(s). Target signaling protein(s)/polypeptide(s) may be tyrosine kinases, such as TTN or BCR, or serine/threonine kinases, or direct substrates of such kinases, or may be indirect substrates downstream of such kinases in signaling pathways. Target signaling protein/polypeptide where elucidated in leukemia cell lines, however one of skill in the art will appreciate that a target signaling protein/polypeptide may also be phosphorylated in other cell lines (non-leukemic) harboring activated kinase activity.

“Heavy-isotope labeled peptide” (used interchangeably with AQUA peptide) means a peptide comprising at least one heavy-isotope label, which is suitable for absolute quantification or detection of a protein as described in WO/03016861, “Absolute Quantification of Proteins and Modified Forms Thereof by Multistage Mass Spectrometry” (Gygi et al.), further discussed below.

“Protein” is used interchangeably with polypeptide, and includes protein fragments and domains as well as whole protein.

“Phosphorylatable amino acid” means any amino acid that is capable of being modified by addition of a phosphate group, and includes both forms of such amino acid.

“Phosphorylatable peptide sequence” means a peptide sequence comprising a phosphorylatable amino acid.

“Phosphorylation site-specific antibody” means an antibody that specifically binds a phosphorylatable peptide sequence/epitope only when phosphorylated, or only when not phosphorylated, respectively. The term is used interchangeably with “phospho-specific” antibody.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 11th Ed., McGraw Hill Companies Inc., New York (2006).

A. Identification of Phosphorylation Sites. The target signaling protein/polypeptide phosphorylation sites disclosed herein and listed in Table 1/FIG. 2 were discovered by employing the modified peptide isolation and characterization techniques described in U.S. Pat. No. 7,198,896 using cellular extracts from the following human cancer cell lines, tissues and patient samples: 01364548-cll, 223-CLL, 293T, 3T3 TrkB, 3T3-Src, 3T3-TrkA, 3T3-wt, 577, A172, AML-4833, AML-6246, AML-6735, AML-7592, BaF3-10ZF, BaF3-4ZF, BaF3-APR, BaF3-FLT3(D842V), BaF3-FLT3(D842Y), BaF3-FLT3(K663Q), BaF3-FLT3(WT), BaF3-FLT3/ITD, BaF3-PRTK, BaF3-TDII, BaF3-Tel/FGFR3, Baf3, Baf3-V617F-jak2, Baf3/E255K, Baf3/H396P, Baf3/Jak2(IL-3 dep), Baf3/M35 IT, Baf3/T315I, Baf3/TpoR, Baf3/TpoR-Y98F, Baf3/Tyk2, Baf3/V617F-jak2 (IL-3), Baf3/Y253F, Baf3/cc-TpoR-IV, Baf3/p210wt, CHRF, CI-1, CMK, CTV-1, DMS 53, DND41, DU-528, DU145, ELF-153, EOL-1, GDM-1, H1703, H1734, H1793, H1869, H1944, H1993, H2023, H226, H3255, H358, H520, H82, H838, HCC1428, HCC1435, HCC1806, HCC1937, HCC366, HCC827, HCT116, HEL, HL107B, HL117B, HL131A, HL131B, HL133A, HL53B, HL59b, HL60, HL61a, HL61b, HL66B, HL68A, HL75A, HL84A, HL97B, HL98A, HT29, HU-3, HUVEC, Jurkat, K562, KG-1, KG1-A, KMS11, KMS18, KMS27, KOFT-K1, KY821, Karpas 299, Karpas-1106p, M-07e, M01043, M059K, MC-116, MCF-10A (Y561F), MCF-10A(Y969F), MDA-MB-453, MDA-MB-468, MEC-2, MKPL-1, ML-1, MO-91, MOLT15, MV4-11, Me-F2, Molm 14, Monomac 6, NCI-N87, Nomo-1, OCI-M1, OCI-ly4, OCI-ly8, OCI/AML2, OPM-1, PL21, Pfeiffer, RC-K8, RI-1, SCLC T1, SEM, SK-N-AS, SK-N-MC, SKBR3, SR-786, SU-DHL1, SUP-M2, SUPT-13, SuDHL5, T17, TRE-cll patient, TS, UT-7, VAL, Verona, Verona 1, Verona 4, WSU-NHL, XG2, Z-55, cs001, cs015, cs025, cs041, cs042, gz21, gz68, gz73, gz74, gzB1, h1144b, h1152b, lung tumor T26, lung tumor T57, normal human lung, pancreatic xenograft, patient 1, rat brain, sw480. The isolation and identification of phosphopeptides from these cell lines, using an immobilized general phosphotyrosine-specific antibody, or an antibody recognizing the phosphorylated motif PXpSP is described in detail in Example 1 below. In addition to the protein phosphorylation sites (tyrosine) described herein, many known phosphorylation sites were also identified (not described herein). The immunoaffinity/mass spectrometric technique described in the '896 Patent (the “IAP” method)—and employed as described in detail in the Examples—is briefly summarized below.

The IAP method employed generally comprises the following steps: (a) a proteinaceous preparation (e.g. a digested cell extract) comprising phosphopeptides from two or more different proteins is obtained from an organism; (b) the preparation is contacted with at least one immobilized general phosphotyrosine-specific antibody; (c) at least one phosphopeptide specifically bound by the immobilized antibody in step (b) is isolated; and (d) the modified peptide isolated in step (c) is characterized by mass spectrometry (MS) and/or tandem mass spectrometry (MS-MS). Subsequently, (e) a search program (e.g., Sequest) may be utilized to substantially match the spectra obtained for the isolated, modified peptide during the characterization of step (d) with the spectra for a known peptide sequence. A quantification step employing, e.g., SILAC or AQUA, may also be employed to quantify isolated peptides in order to compare peptide levels in a sample to a baseline.

In the IAP method as employed herein, a general phosphotyrosine-specific monoclonal antibody (commercially available from Cell Signaling Technology, Inc., Beverly, Mass., Cat. #9411 (p-Tyr-100)) was used in the immunoaffinity step to isolate the widest possible number of phospho-tyrosine containing peptides from the cell extracts.

Extracts from the following human cancer cell lines, tissues and patient samples were employed: 01364548-cll, 223-CLL, 293T, 3T3 TrkB, 3T3-Src, 3T3-TrkA, 3T3-wt, 577, A172, AML-4833, AML-6246, AML-6735, AML-7592, BaF3-10ZF, BaF3-4ZF, BaF3-APR, BaF3-FLT3(D842V), BaF3-FLT3(D842Y), BaF3-FLT3(K663Q), BaF3-FLT3(WT), BaF3-FLT3/ITD, BaF3-PRTK, BaF3-TDII, BaF3-Tel/FGFR3, Baf3, Baf3-V617F-jak2, Baf3/E255K, Baf3/H396P, Baf3/Jak2(IL-3 dep), Baf3/M351T, Baf3/T315I, Baf3/TpoR, Baf3/TpoR-Y98F, Baf3/Tyk2, Baf3/V617F-jak2 (IL-3), Baf3/Y253F, Baf3/cc-TpoR-IV, Baf3/p210wt, CHRF, CI-1, CMK, CTV-1, DMS 53, DND41, DU-528, DU145, ELF-153, EOL-1, GDM-1, H1703, H1734, H1793, H1869, H1944, H1993, H2023, H226, H3255, H358, H520, H82, H838, HCC1428, HCC1435, HCC1806, HCC1937, HCC366, HCC827, HCT116, HEL, HL107B, HL117B, HL131A, HL131B, HL133A, HL53B, HL59b, HL60, HL61a, HL61b, HL66B, HL68A, HL75A, HL84A, HL97B, HL98A, HT29, HU-3, HUVEC, Jurkat, K562, KG-1, KG1-A, KMS11, KMS18, KMS27, KOPT-K1, KY821, Karpas 299, Karpas-1106p, M-07e, M01043, M059K, MC-116, MCF-10A (Y561F), MCF-10A(Y969F), MDA-MB-453, MDA-MB-468, MEC-2, MKPL-1, ML-1, MO-91, MOLT15, MV4-11, Me-F2, Molm 14, Monomac 6, NCI-N87, Nomo-1, OCI-M1, OCI-ly4, OCI-1y8, OCI/AML2, OPM-1, PL21, Pfeiffer, RC-K8, RI-1, SCLC T1, SEM, SK-N-AS, SK-N-MC, SKBR3, SR-786, SU-DHL1, SUP-M2, SUPT-13, SuDHL5, T17, TRE-cll patient, TS, UT-7, VAL, Verona, Verona 1, Verona 4, WSU-NHL, XG2, Z-55, cs001, cs015, cs025, cs041, cs042, gz21, gz68, gz73, gz74, gzB1, h1144b, h1152b, lung tumor T26, lung tumor T57, normal human lung, pancreatic xenograft, patient 1, rat brain and sw480.

As described in more detail in the Examples, lysates were prepared from these cells and digested with trypsin after treatment with DTT and iodoacetamide to redue and alkylate cysteine residues. Before the immunoaffinity step, peptides were pre-fractionated by reversed-phase solid phase extraction using Sep-Pak C₁₈ columns to separate peptides from other cellular components. The solid phase extraction cartridges were eluted with varying steps of acetonitrile. Each lyophilized peptide fraction was redissolved in MOPS IP buffer and treated with phosphotyrosine (P-Tyr-100, CST #9411) immobilized on protein G-Sepharose. Immunoaffinity-purified peptides were eluted with 0.1% TFA and a portion of this fraction was concentrated with Stage or Zip tips and analyzed by LC-MS/MS, using either a LCQ or ThermoFinnigan LTQ ion trap mass spectrometer. Peptides were eluted from a 10 cm×75 μm reversed-phase column with a 45-min linear gradient of acetonitrile. MS/MS spectra were evaluated using the program Sequest with the NCBI human protein database.

This revealed the tyrosine phosphorylation sites in signaling pathways affected by kinase activation or active in leukemia cells. The identified phosphorylation sites and their parent proteins are enumerated in Table 1/FIG. 2. The tyrosine at which phosphorylation occurs is provided in Column D, and the peptide sequence encompassing the phosphorylatable tyrosine residue at the site is provided in Column E. If a phosphorylated tyrosine was found in mouse, the orthologous site in human was identified using either Homologene or BLAST at NCBI; the sequence reported in column E is the phosphorylation site flanked by 7 amino acids on each side. FIG. 2 also shows the particular type of leukemic disease (see Column G) and cell line(s) (see Column F) in which a particular phosphorylation site was discovered.

As a result of the discovery of these phosphorylation sites, phospho-specific antibodies and AQUA peptides for the detection of and quantification of these sites and their parent proteins may now be produced by standard methods, as described below. These new reagents will prove highly useful in, e.g., studying the signaling pathways and events underlying the progression of leukemias and the identification of new biomarkers and targets for diagnosis and treatment of such diseases in a mammal.

The methods of the present invention are intended for use with any mammal that may experience the benefits of the methods of the invention. Foremost among such mammals are humans, although the invention is not intended to be so limited, and is applicable to veterinary uses. Thus, in accordance with the invention, “mammals” or “mammal in need” include humans as well as non-human mammals, particularly domesticated animals including, without limitation, cats, dogs, and horses.

B. Antibodies and Cell Lines. Isolated phosphorylation site-specific antibodies that specifically bind a target signaling protein/polypeptide disclosed in Column A of Table 1 only when phosphorylated (or only when not phosphorylated) at the corresponding amino acid and phosphorylation site listed in Columns D and E of Table 1/FIG. 2 may be produced by standard antibody production methods, such as anti-peptide antibody methods, using the phosphorylation site sequence information provided in Column E of Table 1. The TAGLN3 cytoskeletal protein phosphorylation site (tyrosine 192) (see Row 66 of Table 1/FIG. 2) is presently disclosed. Thus, an antibody that specifically binds this novel TAGLN3 cytoskeletal site can now be produced, e.g. by immunizing an animal with a peptide antigen comprising all or part of the amino acid sequence encompassing the respective phosphorylated residue (e.g., a peptide antigen comprising the sequence set forth in Row 66, Column E, of Table 1, SEQ ID NO: 65, respectively) (which encompasses the phosphorylated tyrosine at position 192 in TAGLN3, to produce an antibody that only binds TAGLN3 cytoskeletal protein when phosphorylated at that site.

Polyclonal antibodies of the invention may be produced according to standard techniques by immunizing a suitable animal (e.g., rabbit, goat, etc.) with a peptide antigen corresponding to the phosphorylation site of interest (i.e., a phosphorylation site enumerated in Column E of Table 1, which comprises the corresponding phosphorylatable amino acid listed in Column D of Table 1), collecting immune serum from the animal, and separating the polyclonal antibodies from the immune serum, in accordance with known procedures. For example, a peptide antigen corresponding to all or part of the novel TES cytoskeletal phosphorylation site disclosed herein (SEQ ID NO: 71=RTQYSCyCCK, encompassing phosphorylated tyrosine 237 (see Row 72 of Table 1)) may be employed to produce antibodies that only bind TES when phosphorylated at Tyr 237. Similarly, a peptide comprising all or part of any one of the phosphorylation site sequences provided in Column E of Table 1 may employed as an antigen to produce an antibody that only binds the corresponding protein listed in Column A of Table 1 when phosphorylated (or when not phosphorylated) at the corresponding residue listed in Column D. If an antibody that only binds the protein when phosphorylated at the disclosed site is desired, the peptide antigen includes the phosphorylated form of the amino acid. Conversely, if an antibody that only binds the protein when not phosphorylated at the disclosed site is desired, the peptide antigen includes the non-phosphorylated form of the amino acid.

Peptide antigens suitable for producing antibodies of the invention may be designed, constructed and employed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)).

It will be appreciated by those of skill in the art that longer or shorter phosphopeptide antigens may be employed. See Id. For example, a peptide antigen may comprise the full sequence disclosed in Column E of Table 1/FIG. 2, or it may comprise additional amino acids flanking such disclosed sequence, or may comprise of only a portion of the disclosed sequence immediately flanking the phosphorylatable amino acid (indicated in Column E by lowercase “y”). Typically, a desirable peptide antigen will comprise four or more amino acids flanking each side of the phosphorylatable amino acid and encompassing it. Polyclonal antibodies produced as described herein may be screened as further described below.

Monoclonal antibodies of the invention may be produced in a hybridoma cell line according to the well-known technique of Kohler and Milstein. See Nature 265: 495-97 (1975); Kohler and Milstein, Eur. J. Immunol. 6: 511 (1976); see also, Current Protocols in Molecular Biology, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal F_{ab} fragments may also be produced in Escherichia coli by recombinant techniques known to those skilled in the art. See, e.g., W. Huse, Science 246: 1275-81 (1989); Mullinax et al., Proc. Nat'l Acad. Sci. 87: 8095 (1990). If monoclonal antibodies of one isotype are preferable for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

An epitope of a phosphorylation-site specific antibody of the invention is a peptide fragment consisting essentially of about 8 to 17 amino acids including the phosphorylatable tyrosine, wherein about 3 to 8 amino acids are positioned on each side of the phosphorylatable tyrosine (for example, the WDR1 tyrosine 98 phosphorylation site sequence disclosed in Row 83, Column E of Table 1), and antibodies of the invention thus specifically bind a target signal protein/polypepetide comprising such epitopic sequence. Epitopes bound by the antibodies of the invention comprise all or part of a phosphorylatable site sequence listed in Column E of Table 1, including the phosphorylatable amino acid.

Included in the scope of the invention are equivalent non-antibody molecules, such as protein binding domains or nucleic acid aptamers, which bind, in a phospho-specific manner, to essentially the same phosphorylatable epitope to which the phospho-specific antibodies of the invention bind. See, e.g., Neuberger et al., Nature 312: 604 (1984). Such equivalent non-antibody reagents may be suitably employed in the methods of the invention further described below.

Antibodies provided by the invention may be any type of immunoglobulins, including IgG, IgM, IgA, IgD, and IgE, including F_{ab} or antigen-recognition fragments thereof. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 or U.S. Pat. No. 4,816,567. The antibodies may also be chemically constructed by specific antibodies made according to the method disclosed in U.S. Pat. No. 4,676,980.

The invention also provides immortalized cell lines that produce an antibody of the invention. For example, hybridoma clones, constructed as described above, that produce monoclonal antibodies to the protein phosphorylation sites disclosed herein are also provided. Similarly, the invention includes recombinant cells producing an antibody of the invention, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Phosphorylation site-specific antibodies of the invention, whether polyclonal or monoclonal, may be screened for epitope and phospho-specificity according to standard techniques. See, e.g. Czernik et al., Methods in Enzymology, 201: 264-283 (1991). For example, the antibodies may be screened against the phospho and non-phospho peptide library by ELISA to ensure specificity for both the desired antigen (i.e. that epitope including a phosphorylation site sequence enumerated in Column E of Table 1) and for reactivity only with the phosphorylated (or non-phosphorylated) form of the antigen. Peptide competition assays may be carried out to confirm lack of reactivity with other phospho-epitopes on the given target signal protein/polypepetide. The antibodies may also be tested by Western blotting against cell preparations containing the signaling protein, e.g. cell lines over-expressing the target protein, to confirm reactivity with the desired phosphorylated epitope/target.

In an exemplary embodiment, phage display libraries containing more than 10¹⁰ phage clones are used for high-throughput production of monoclonal antibodies that target post-translational modification sites (e.g., phosphorylation sites) and, for validation and quality control, high-throughput immunohistochemistry is utilized to screen the efficacy of these antibodies. Western blots, protein microarrays and flow cytometry can also be used in high-throughput screening of phosphorylation site-specific polyclonal or monoclonal antibodies of the present invention. See, e.g., Blow N., Nature, 447: 741-743 (2007).

Specificity against the desired phosphorylated epitope may also be examined by constructing mutants lacking phosphorylatable residues at positions outside the desired epitope that are known to be phosphorylated, or by mutating the desired phospho-epitope and confirming lack of reactivity. Phosphorylation-site specific antibodies of the invention may exhibit some limited cross-reactivity to related epitopes in non-target proteins. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-target proteins is readily characterized by Western blotting alongside markers of known molecular weight. Amino acid sequences of cross-reacting proteins may be examined to identify sites highly homologous to the Target signaling protein/polypeptide epitope for which the antibody of the invention is specific.

In certain cases, polyclonal antisera may exhibit some undesirable general cross-reactivity to phosphotyrosine or phosphoserine itself, which may be removed by further purification of antisera, e.g., over a phosphotyramine column. Antibodies of the invention specifically bind their target protein (i.e., a protein listed in Column A of Table 1) only when phosphorylated (or only when not phosphorylated, as the case may be) at the site disclosed in corresponding Columns D/E, and do not (substantially) bind to the other form (as compared to the form for which the antibody is specific).

Antibodies may be further characterized via immunohistochemical (IHC) staining using normal and diseased tissues to evaluate phosphorylation and activation status in diseased tissue. IHC may be carried out according to well-known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g., tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies may be further characterized by flow cytometry carried out according to standard methods. See Chow et al., Cytometry (Communications in Clinical Cytometry) 46: 72-78 (2001). Briefly and by way of example, the following protocol for cytometric analysis may be employed: samples may be centrifuged on Ficoll gradients to remove erythrocytes, and cells may then be fixed with 2% paraformaldehyde for 10 minutes at 37° C. followed by permeabilization in 90% methanol for 30 minutes on ice. Cells may then be stained with the primary phosphorylation-site specific antibody of the invention (which detects a target signal protein/polypepetide enumerated in Table 1), washed and labeled with a fluorescent-labeled secondary antibody. Additional fluorochrome-conjugated marker antibodies (e.g., CD45, CD34) may also be added at this time to aid in the subsequent identification of specific hematopoietic cell types. The cells would then be analyzed on a flow cytometer (e.g., a Beckman Coulter FC500) according to the specific protocols of the instrument used.

Antibodies of the invention may also be advantageously conjugated to fluorescent dyes (e.g., Alexa488, PE) for use in multi-parametric analyses along with other signal transduction (phospho-CrkL, phospho-Erk 1/2) and/or cell marker (CD34) antibodies.

Phosphorylation-site specific antibodies of the invention specifically bind to a target signaling protein/polypeptide only when phosphorylated at a disclosed site, but are not limited only to binding the human species, per se. The invention includes antibodies that also bind conserved and highly homologous or identical phosphorylation sites in respective Target signaling protein/polypeptide from other species (e.g., mouse, rat, monkey, yeast), in addition to binding the human phosphorylation site. Highly homologous or identical sites conserved in other species can readily be identified by standard sequence comparisons, such as using BLAST, with the human Target signaling protein/polypeptide phosphorylation sites disclosed herein.

C. Heavy-Isotope Labeled Peptides (AQUA Peptides). The phosphorylation sites disclosed herein now enable the production of corresponding heavy-isotope labeled peptides for the absolute quantification of such signaling proteins (both phosphorylated and not phosphorylated at a disclosed site) in biological samples. The production and use of AQUA peptides for the absolute quantification of proteins (AQUA) in complex mixtures has been described. See WO/03016861, Gerber et al., Proc. Natl. Acad. Sci. U.S.A. 100: 6940-5 (2003).

The AQUA methodology employs the introduction of a known quantity of at least one heavy-isotope labeled peptide standard (which has a unique signature detectable by LC-SRM chromatography) into a digested biological sample in order to determine, by comparison to the peptide standard, the absolute quantity of a peptide with the same sequence and protein modification in the biological sample. Briefly, the AQUA methodology has two stages: peptide internal standard selection and validation and method development; and implementation using validated peptide internal standards to detect and quantify a target protein in sample. The method is a powerful technique for detecting and quantifying a given peptide/protein within a complex biological mixture, such as a cell lysate, and may be employed, e.g., to quantify change in protein phosphorylation as a result of drug treatment, or to quantify differences in the level of a protein in different biological states.

Generally, to develop a suitable internal standard, a particular peptide (or modified peptide) within a target protein sequence is chosen based on its amino acid sequence and the particular protease to be used to digest. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a 7-Da mass shift. A newly synthesized AQUA internal standard peptide is then evaluated by LC MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or modified protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis. (See Gerber et al., supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g., trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures. Because an absolute amount of the AQUA peptide is added (e.g., 250 fmol), the ratio of the areas under the curve can be used to determine the precise expression levels of a protein or phosphorylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard is developed for a known phosphorylation site sequence previously identified by the IAP-LC-MS/MS method within a target protein. One AQUA peptide incorporating the phosphorylated form of the particular residue within the site may be developed, and a second AQUA peptide incorporating the non-phosphorylated form of the residue developed. In this way, the two standards may be used to detect and quantify both the phosphorylated and non-phosphorylated forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, serine proteases (e.g., trypsin, hepsin), metallo proteases (e.g., PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. A workable range is about 7 to 15 amino acids. A peptide sequence is also selected that is not likely to be chemically reactive during mass spectrometry, thus sequences comprising cysteine, tryptophan, or methionine are avoided.

A peptide sequence that does not include a modified region of the target region may be selected so that the peptide internal standard can be used to determine the quantity of all forms of the protein. Alternatively, a peptide internal standard encompassing a modified amino acid may be desirable to detect and quantify only the modified form of the target protein. Peptide standards for both modified and unmodified regions can be used together, to determine the extent of a modification in a particular sample (i.e. to determine what fraction of the total amount of protein is represented by the modified form). For example, peptide standards for both the phosphorylated and unphosphorylated form of a protein known to be phosphorylated at a particular site can be used to quantify the amount of phosphorylated form in a sample.

The peptide is labeled using one or more labeled amino acids (i.e. the label is an actual part of the peptide) or less preferably, labels may be attached after synthesis according to standard methods. Preferably, the label is a mass-altering label selected based on the following considerations: the mass should be unique to shift fragment masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the 20 natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ²H, ¹³C, ¹⁵N, ¹⁷O, ¹⁸O, or ³⁴S, are sutable labels. Pairs of peptide internal standards that incorporate a different isotope label may also be prepared. Amino acid residues into which a heavy isotope label may be incorporated include leucine, proline, valine, and phenylalanine.

Peptide internal standards are characterized according to their mass-to-charge (m/z) ratio, and preferably, also according to their retention time on a chromatographic column (e.g. an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts may be employed. Generally, the sample has at least 0.01 mg of protein, typically a concentration of 0.1-10 mg/mL, and may be adjusted to a desired buffer concentration and pH.

A known amount of a labeled peptide internal standard, preferably about 10 femtomoles, corresponding to a target protein to be detected/quantified is then added to a biological sample, such as a cell lysate. The spiked sample is then digested with one or more protease(s) for a suitable time period to allow digestion. A separation is then performed (e.g., by HPLC, reverse-phase HPLC, capillary electrophoresis, ion exchange chromatography, etc.) to isolate the labeled internal standard and its corresponding target peptide from other peptides in the sample. Microcapillary LC is a method contemplated.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

In accordance with the present invention, AQUA internal peptide standards (heavy-isotope labeled peptides) may now be produced, as described above, for any of the phosphorylation sites disclosed herein. Peptide standards for a given phosphorylation site (e.g., the tyrosine 328 in TOP2A—see Row 87 of Table 1) may be produced for both the phosphorylated and non-phosphorylated forms of the site (e.g., see PKCD site sequence in Column E, Row 123 of Table 1 (SEQ ID NO: 122) and such standards employed in the AQUA methodology to detect and quantify both forms of such phosphorylation site in a biological sample.

AQUA peptides of the invention may comprise all, or part of, a phosphorylation site peptide sequence disclosed herein (see Column E of Table 1/FIG. 2). In an embodiment, an AQUA peptide of the invention comprises a phosphorylation site sequence disclosed herein in Table 1/FIG. 2. For example, an AQUA peptide of the invention for detection/quantification of SMRT transcriptional regulator protein when phosphorylated at tyrosine Y2249 may comprise the sequence SAVyPLLYR (y=phosphotyrosine), which comprises phosphorylatable tyrosine 2249 (see Row 177, Column E; (SEQ ID NO: 176)). Heavy-isotope labeled equivalents of the peptides enumerated in Table 1/FIG. 2 (both in phosphorylated and unphosphorylated form) can be readily synthesized and their unique MS and LC-SRM signature determined, so that the peptides are validated as AQUA peptides and ready for use in quantification experiments.

The phosphorylation site peptide sequences disclosed herein (see Column E of Table 1/FIG. 2) are well suited for development of corresponding AQUA peptides, since the IAP method by which they were identified (see Part A above and Example 1) inherently confirmed that such peptides are in fact produced by enzymatic digestion (trypsinization) and are in fact suitably fractionated/ionized in MS/MS. Thus, heavy-isotope labeled equivalents of these peptides (both in phosphorylated and unphosphorylated form) can be readily synthesized and their unique MS and LC-SRM signature determined, so that the peptides are validated as AQUA peptides and ready for use in quantification experiments.

Accordingly, the invention provides heavy-isotope labeled peptides (AQUA peptides) for the detection and/or quantification of any of the phosphorylation sites disclosed in Table 1/FIG. 2 (see Column E) and/or their corresponding parent proteins/polypeptides (see Column A). A phosphopeptide sequence comprising any of the phosphorylation sequences listed in Table 1 may be considered an AQUA peptide of the invention. For example, an AQUA peptide comprising the sequence SAVyPLLYR (SEQ ID NO: 176) (where y may be either phosphotyrosine or tyrosine, and where V=labeled valine (e.g., ¹⁴C)) is provided for the quantification of phosphorylated (or non-phosphorylated) diaphanous (Tyr2249) in a biological sample (see Row 177 of Table 1, tyrosine 2249 being the phosphorylatable residue within the site). It will be appreciated that a larger AQUA peptide comprising a disclosed phosphorylation site sequence (and additional residues downstream or upstream of it) may also be constructed. Similarly, a smaller AQUA peptide comprising less than all of the residues of a disclosed phosphorylation site sequence (but still comprising the phosphorylatable residue enumerated in Column D of Table 1/FIG. 2) may alternatively be constructed. Such larger or shorter AQUA peptides are within the scope of the present invention, and the selection and production of AQUA peptides may be carried out as described above (see Gygi et al., Gerber et al., supra.).

Certain subsets of AQUA peptides provided by the invention are described above (corresponding to particular protein types/groups in Table 1, for example, tyrosine protein kinases or adaptor/scaffold proteins). Example 4 is provided to further illustrate the construction and use, by standard methods described above, of exemplary AQUA peptides provided by the invention. For example, the above-described AQUA peptides corresponding to both the phosphorylated and non-phosphorylated forms of the disclosed Tel transcriptional regulator protein tyrosine 402 phosphorylation site (see Row 213 of Table 1/FIG. 2) may be used to quantify the amount of phosphorylated Tel (Tyr 402) in a biological sample, e.g., a tumor cell sample (or a sample before or after treatment with a test drug).

AQUA peptides of the invention may also be employed within a kit that comprises one or multiple AQUA peptide(s) provided herein (for the quantification of a Target signaling protein/polypeptide disclosed in Table 1/FIG. 2), and, optionally, a second detecting reagent conjugated to a detectable group. For example, a kit may include AQUA peptides for both the phosphorylated and non-phosphorylated form of a phosphorylation site disclosed herein. The reagents may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

AQUA peptides provided by the invention will be useful in the further study of signal transduction anomalies associated with diseases such as for example cancer, including leukemias, and in identifying diagnostic/bio-markers of these diseases, new potential drug targets, and/or in monitoring the effects of test compounds on Target Signaling Proteins/Polypeptides and pathways.

D. Immunoassay Formats. Antibodies provided by the invention may be advantageously employed in a variety of standard immunological assays (the use of AQUA peptides provided by the invention is described separately above). Assays may be homogeneous assays or heterogeneous assays. In a homogeneous assay the immunological reaction usually involves a phosphorylation-site specific antibody of the invention), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, a phosphorylation-site specific antibody of the invention, and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof that may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No. 4,727,022; U.S. Pat. No. 4,659,678; U.S. Pat. No. 4,376,110. Conditions suitable for the formation of reagent-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of a Target signaling protein/polypeptide is detectable compared to background.

Phosphorylation site-specific antibodies disclosed herein may be conjugated to a solid support suitable for a diagnostic assay (e.g., beads, plates, slides or wells formed from materials such as latex or polystyrene) in accordance with known techniques, such as precipitation. Antibodies, or other target protein or target site-binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

Antibodies of the invention may also be optimized for use in a flow cytometry (FC) assay to determine the activation/phosphorylation status of a Target signaling protein/polypeptide in patients before, during, and after treatment with a drug targeted at inhibiting phosphorylation of such a protein at the phosphorylation site disclosed herein. For example, bone marrow cells or peripheral blood cells from patients may be analyzed by flow cytometry for Target signaling protein/polypeptide phosphorylation, as well as for markers identifying various hematopoietic cell types. In this manner, activation status of the malignant cells may be specifically characterized. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 1% para-formaldehyde for 10 minutes at 37° C. followed by permeabilization in 90% methanol for 30 minutes on ice. Cells may then be stained with the primary antibody (a phospho-specific antibody of the invention), washed and labeled with a fluorescent-labeled secondary antibody. Alternatively, the cells may be stained with a fluorescent-labeled primary antibody. The cells would then be analyzed on a flow cytometer (e.g., a Beckman Coulter EPICS-XL) according to the specific protocols of the instrument used. Such an analysis would identify the presence of activated Target Signaling Protein(s)/Polypeptide(s) in the malignant cells and reveal the drug response on the targeted protein.

Alternatively, antibodies of the invention may be employed in immunohistochemical (IHC) staining to detect differences in signal transduction or protein activity using normal and diseased tissues. IHC may be carried out according to well-known techniques. See, e.g., Antibodies: A Laboratory Manual, supra. Briefly, paraffin-embedded tissue (e.g., tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies of the invention may be also be optimized for use in other clinically-suitable applications, for example bead-based multiplex-type assays, such as IGEN, Luminex™ and/or Bioplex™ assay formats, or otherwise optimized for antibody array formats, such as reversed-phase array applications (see, e.g., Paweletz et al., Oncogene 20(16): 1981-89 (2001)). Accordingly, in another embodiment, the invention provides a method for the multiplex detection of phosphorylation in a biological sample, the method comprising utilizing two or more antibodies or AQUA peptides of the invention to detect the presence of two or more phosphorylated proteins enumerated in Column A of Table 1/FIG. 2. In an embodiment, two to five antibodies or AQUA peptides of the invention are employed in the method. In another embodiment, six to ten antibodies or AQUA peptides of the invention are employed, while in another embodiment eleven to twenty such reagents are employed.

Antibodies and/or AQUA peptides of the invention may also be employed within a kit that comprises at least one phosphorylation site-specific antibody or AQUA peptide of the invention (which binds to or detects a Target signaling protein/polypeptide disclosed in Table 1/FIG. 2), and, optionally, a second antibody conjugated to a detectable group. In some embodies, the kit is suitable for multiplex assays and comprises two or more antibodies or AQUA peptides of the invention, and in some embodiments, comprises two to five, six to ten, or eleven to twenty reagents of the invention. The kit may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the description, numerous specific details are set forth in order to provide a thorough understanding of the present invention. The present invention may be practiced without some or all of these specific details. In other instances, well known process operations have not been described in detail, in order not to unnecessarily obscure the present invention.

The following examples are intended to further illustrate certain embodiments of the invention and are not limiting in nature. Those skilled in the art will recognize, or be able to ascertain, using no more than routine experimentation, numerous equivalents to the specific substances and procedures described herein.

Any suitable materials and/or methods known to those of skill can be utilized in carrying out the present invention. However, materials and methods are described. Materials, reagents and the like to which reference is made in the following description and examples are obtainable from commercial sources, unless otherwise noted.

### Example 1

### Isolation of Phosphotyrosine-Containing Peptides from Extracts of Cancer Cell Lines and Identification of Phosphorylation Sites

IAP isolation techniques were employed to identify phosphotyrosine containing peptides in cell extracts from the following human cancer cell lines, tissues and patient cell lines: 01364548-cll, 223-CLL, 293T, 3T3 TrkB, 3T3-Src, 3T3-TrkA, 3T3-wt, 577, A 172, AML-4833, AML-6246, AML-6735, AML-7592, BaF3-10ZF, BaF3-4ZF, BaF3-APR, BaF3-FLT3(D842V), BaF3-FLT3(D842Y), BaF3-FLT3(K663Q), BaF3-FLT3(WT), BaF3-FLT3/ITD, BaF3-PRTK, BaF3-TDII, BaF3-Tel/FGFR3, Baf3, Baf3-V617F-jak2, Baf3/E255K, Baf3/H396P, Baf3/Jak2(IL-3 dep), Baf3/M351T, Baf3/T315I, Baf3/TpoR, Baf3/TpoR-Y98F, Baf3/Tyk2, Baf3/V617F-jak2 (IL-3), Baf3/Y253F, Baf3/cc-TpoR-IV, Baf3/p210wt, CHRF, CI-1, CMK, CTV-1, DMS 53, DND41, DU-528, DU145, ELF-153, EOL-1, GDM-1, H1703, H1734, H1793, H1869, H1944, H1993, H2023, H226, H3255, H358, H520, H82, H838, HCC1428, HCC1435, HCC1806, HCC1937, HCC366, HCC827, HCT116, HEL, HL107B, HL117B, HL131A, HL131B, HL133A, HL53B, HL59b, HL60, HL61a, HL61b, HL66B, HL68A, HL75A, HL84A, HL97B, HL98A, HT29, HU-3, HUVEC, Jurkat, K562, KG-1, KG1-A, KMS11, KMS18, KMS27, KOPT-K 1, KY821, Karpas 299, Karpas-1106p, M-07e, M01043, M059K, MC-116, MCF-10A (Y561F), MCF-10A(Y969F), MDA-MB-453, MDA-MB-468, MEC-2, MKPL-1, ML-1, MO-91, MOLT15, MV4-11, Me-F2, Molm 14, Monomac 6, NCI-N87, Nomo-1, OCI-M1, OCI-ly4, OCI-1y8, OCI/AML2, OPM-1, PL21, Pfeiffer, RC-K8, RI-1, SCLC T1, SEM, SK-N-AS, SK-N-MC, SKBR3, SR-786, SU-DHL1, SUP-M2, SUPT-13, SuDHL5, T17, TRE-cll patient, TS, UT-7, VAL, Verona, Verona 1, Verona 4, WSU-NHL, XG2, Z-55, cs001, cs015, cs025, cs041, cs042, gz21, gz68, gz73, gz74, gzB1, h1144b, hl 152b, lung tumor T26, lung tumor T57, normal human lung, pancreatic xenograft, patient 1, rat brain and sw480.

Tryptic phosphotyrosine containing peptides were purified and analyzed from extracts of each of the cell lines mentioned above, as follows. Cells were cultured in DMEM medium or RPMI 1640 medium supplemented with 10% fetal bovine serum and penicillin/streptomycin.

Suspension cells were harvested by low speed centrifugation. After complete aspiration of medium, cells were resuspended in 1 mL lysis buffer per 1.25×10⁸ cells (20 mM HEPES pH 8.0, 9 M urea, 1 mM sodium vanadate, supplemented or not with 2.5 mM sodium pyro-phosphate, 1 mM β-glycerol-phosphate) and sonicated.

Sonicated cell lysates were cleared by centrifugation at 20,000×g, and proteins were reduced with DTT at a final concentration of 4.1 mM and alkylated with iodoacetamide at 8.3 mM. For digestion with trypsin, protein extracts were diluted in 20 mM HEPES pH 8.0 to a final concentration of 2 M urea and soluble TLCK®-trypsin (Worthington® Biochemcial Corporation, Lakewood, N.J.) was added at 10-20 μg/mL. Digestion was performed for 1-2 days at room temperature.

Trifluoroacetic acid (TFA) was added to protein digests to a final concentration of 1%, precipitate was removed by centrifugation, and digests were loaded onto Sep-Pak® C₁₈ columns (provided by Waters Corporation, Milford, Mass.) equilibrated with 0.1% TFA. A column volume of 0.7-1.0 ml was used per 2×10⁸ cells. Columns were washed with 15 volumes of 0.1% TFA, followed by 4 volumes of 5% acetonitrile (MeCN) in 0.1% TFA. Peptide fraction I was obtained by eluting columns with 2 volumes each of 8, 12, and 15% MeCN in 0.1% TFA and combining the eluates. Fractions II and III were a combination of eluates after eluting columns with 18, 22, 25% MeCN in 0.1% TFA and with 30, 35, 40% MeCN in 0.1% TFA, respectively. All peptide fractions were lyophilized.

Peptides from each fraction corresponding to 2×10⁸ cells were dissolved in 1 ml of IAP buffer (20 mM Tris/HCl or 50 mM MOPS pH 7.2, 10 mM sodium phosphate, 50 mM NaCl) and insoluble material was removed by centrifugation. IAP was performed on each peptide fraction separately. The phosphotyrosine monoclonal antibody P-Tyr-100 (Cell Signaling Technology®, Inc., Danvers, Mass. catalog number 9411) was coupled at 4 mg/ml beads to protein G or protein A agarose (Roche®, Basel, Switzerland), respectively. Immobilized antibody (15 μl, 60 μg) was added as 1:1 slurry in IAP buffer to 1.4 ml of each peptide fraction, and the mixture was incubated overnight at 4° C. with gentle rotation. The immobilized antibody beads were washed three times with 1 ml IAP buffer and twice with 1 ml water, all at 4° C. Peptides were eluted from beads by incubation with 75 μl of 0.1% TFA at room temperature for 10 minutes.

Alternatively, one single peptide fraction was obtained from Sep-Pak C18 columns by elution with 2 volumes each of 10%, 15%, 20%, 25%, 30%, 35% and 40% acetonitirile in 0.1% TFA and combination of all eluates. IAP on this peptide fraction was performed as follows: After lyophilization, peptide was dissolved in 1.4 ml IAP buffer (MOPS pH 7.2, 10 mM sodium phosphate, 50 mM NaCl) and insoluble material was removed by centrifugation. Immobilized antibody (40 μl, 160 μg) was added as 1:1 slurry in IAP buffer, and the mixture was incubated overnight at 4° C. with gentle shaking. The immobilized antibody beads were washed three times with 1 ml IAP buffer and twice with 1 ml water, all at 4° C. Peptides were eluted from beads by incubation with 40 μl of 0.15% TFA at room temperature for 10 min (eluate 1), followed by a wash of the beads (eluate 2) with 40 μl of 0.15% TFA. Both eluates were combined.

### Analysis by LC-MS/MS Mass Spectrometry.

40 μl or more of IAP eluate were purified by 0.2 μl StageTips (Proxeon, Staermosegaardsvej 6, DK-5230 Odense M, Denmark) or ZipTips® (produced by Millipore®, Billerica Mass.). Peptides were eluted from the microcolumns with 1 μl of 40% MeCN, 0.1% TFA (fractions I and II) or 1 μl of 60% MeCN, 0.1% TFA (fraction III) into 7.6 μl of 0.4% acetic acid/0.005% heptafluorobutyric acid. This sample was loaded onto a 10 cm×75 μm PicoFrit® capillary column (produced by New Objective, Woburn, Mass.) packed with Michrom Magic Bullets® C18 AQ reversed-phase resin (Michrom Bioresources, Auburn Calif.) using a Famos™ autosampler with an inert sample injection valve (Dionex®, Sunnyvale, Calif.). The column was then developed with a 45-min linear gradient of acetonitrile delivered at 200 nl/min (using an Ultimate® pump, Dionex®, Sunnyvale, Calif.), and tandem mass spectra were collected in a data-dependent manner with an LTQ® (produced by Thermo® Finnigan® San, Jose, Calif.), ion trap mass spectrometer essentially as described by Gygi et al., supra.

### Database Analysis & Assignments.

MS/MS spectra were evaluated using TurboSequest™ in the Sequest® (owned by Thermo® Finnigan® San Jose, Calif.) Browser package (v. 27, rev. 12) supplied as part of BioWorks™ 3.0 (Thermo® Finnigan®, San Jose, Calif.). Individual MS/MS spectra were extracted from the raw data file using the Sequest® Browser program CreateDta™ (owned by Thermo® Finnigan® San Jose, Calif.), with the following settings: bottom MW, 700; top MW, 4,500; minimum number of ions, 20; minimum TIC, 4×10⁵; and precursor charge state, unspecified. Spectra were extracted from the beginning of the raw data file before sample injection to the end of the eluting gradient. The IonQuest™ and VuDta™ (owned by Thermo® Finnigan® San Jose, Calif.) programs were not used to further select MS/MS spectra for Sequest® analysis. MS/MS spectra were evaluated with the following TurboSequest™ parameters: peptide mass tolerance, 2.5; fragment ion tolerance, 0.0; maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

Searches were performed against the NCBI human protein database (as released on Aug. 24, 2004 and containing 27, 960 protein sequences). Cysteine carboxamidomethylation was specified as a static modification, and phosphorylation was allowed as a variable modification on serine, threonine, and tyrosine residues or on tyrosine residues alone. It was determined that restricting phosphorylation to tyrosine residues had little effect on the number of phosphorylation sites assigned. Furthermore, it should be noted that certain peptides were originally isolated in mouse and later normalized to human sequences as shown by Table 1/FIG. 2.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al., Mol. Cell. Proteomics 3: 531-533 (2004)), which were followed in this Example. However, because the immunoaffinity strategy separates phosphorylated peptides from unphosphorylated peptides, observing just one phosphopeptide from a protein is a common result, since many phosphorylated proteins have only one tyrosine-phosphorylated site. For this reason, it is appropriate to use additional criteria to validate phosphopeptide assignments. Assignments are likely to be correct if any of these additional criteria are met: (i) the same sequence is assigned to co-eluting ions with different charge states, since the MS/MS spectrum changes markedly with charge state; (ii) the site is found in more than one peptide sequence context due to sequence overlaps from incomplete proteolysis or use of proteases other than trypsin; (iii) the site is found in more than one peptide sequence context due to homologous but not identical protein isoforms; (iv) the site is found in more than one peptide sequence context due to homologous but not identical proteins among species; and (v) sites validated by MS/MS analysis of synthetic phosphopeptides corresponding to assigned sequences, since the ion trap mass spectrometer produces highly reproducible MS/MS spectra. The last criterion is routinely employed to confirm novel site assignments of particular interest.

All spectra and all sequence assignments made by Sequest were imported into a relational database. The following Sequest scoring thresholds were used to select phosphopeptide assignments that are likely to be correct: RSp<6, XCorr≧2.2, and DeltaCN>0.099. Further, the assigned sequences could be accepted or rejected with respect to accuracy by using the following conservative, two-step process.

In the first step, a subset of high-scoring sequence assignments should be selected by filtering for XCorr values of at least 1.5 for a charge state of +1, 2.2 for +2, and 3.3 for +3, allowing a maximum RSp value of 10. Assignments in this subset should be rejected if any of the following criteria were satisfied: (i) the spectrum contains at least one major peak (at least 10% as intense as the most intense ion in the spectrum) that can not be mapped to the assigned sequence as an a, b, or y ion, as an ion arising from neutral-loss of water or ammonia from a b or y ion, or as a multiply protonated ion; (ii) the spectrum does not contain a series of b or y ions equivalent to at least six uninterrupted residues; or (iii) the sequence is not observed at least five times in all the studies conducted (except for overlapping sequences due to incomplete proteolysis or use of proteases other than trypsin).

In the second step, assignments with below-threshold scores should be accepted if the low-scoring spectrum shows a high degree of similarity to a high-scoring spectrum collected in another study, which simulates a true reference library-searching strategy.

### Example 2

### Production of Phospho-Specific Polyclonal Antibodies for the Detection of Target Signal Protein/Polypepetide Phosphorylation

Polyclonal antibodies that specifically bind a target signal protein/polypepetide only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, as further described below. Production of exemplary polyclonal antibodies is provided below.

### A. VCP (Tyrosine 644).

A 13 amino acid phospho-peptide antigen, LDKLIy*IPLPDEK (where y*=phosphotyrosine) that corresponds to the sequence encompassing the tyrosine 644 phosphorylation site in human VCP cell cycle regulation protein (see Row 28 of Table 1; SEQ ID NO: 27), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific VCP (tyr643) polyclonal antibodies as described in Immunization/Screening below.

### B. HSP90B (Tyrosine 192).

An 16 amino acid phospho-peptide antigen, VILHLKEDQTEy*LEER (where y*=phosphotyrosine) that corresponds to the sequence encompassing the tyrosine 192 phosphorylation site in human HSP90B chaperone protein (see Row 30 of Table 1 (SEQ ID NO: 29)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific HSP90B (tyr 191) polyclonal antibodies as described in Immunization/Screening below.

### C. TSN (Tyrosine 210).

A 12 amino acid phospho-peptide antigen, KVEEVVy*DLSIR (where y*=phosphotyrosine) that corresponds to the sequence encompassing the tyrosine 210 phosphorylation site in human catalase chromatin or DNA binding/repair/replication protein (see Row 44 of Table 1 (SEQ ID NO: 43), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific TSN (tyr 210) antibodies as described in Immunization/Screening below.

### Immunization/Screening.

A synthetic phospho-peptide antigen as described in A-C above is coupled to KLH, and rabbits are injected intradermally (ID) on the back with antigen in complete Freunds adjuvant (384 μg antigen per rabbit). The rabbits are boosted with same antigen in incomplete Freund adjuvant (250 μg antigen per rabbit) every three weeks. After the fifth boost, bleeds are collected. The sera are purified by Protein A-affinity chromatography by standard methods (see ANTIBODIES: A LABORATORY MANUAL, Cold Spring Harbor, supra.). The eluted immunoglobulins are further loaded onto a non-phosphorylated synthetic peptide antigen-resin Knotes column to pull out antibodies that bind the non-phosphorylated form of the phosphorylation site. The flow through fraction is collected and applied onto a phospho-synthetic peptide antigen-resin column to isolate antibodies that bind the phosphorylated form of the site. After washing the column extensively, the bound antibodies (i.e. antibodies that bind a phosphorylated peptide described in A-C above, but do not bind the non-phosphorylated form of the peptide) are eluted and kept in antibody storage buffer.

The isolated antibody is then tested for phospho-specificity using Western blot assay using an appropriate cell line that expresses (or overexpresses) target phospho-protein (i.e. phosphorylated VCP, HSP90B or TSN), for example, CTV, CMK and MOLT15 cells, respectively. Cells are cultured in DMEM or RPMI supplemented with 10% FCS. Cell are collected, washed with PBS and directly lysed in cell lysis buffer. The protein concentration of cell lysates is then measured. The loading buffer is added into cell lysate and the mixture is boiled at 100° C. for 5 minutes. 20 μl (10 μg protein) of sample is then added onto 7.5% SDS-PAGE gel.

A standard Western blot may be performed according to the Immunoblotting Protocol set out in the CELL SIGNALING TECHNOLOGY, INC. 2003-04 Catalogue, p. 390. The isolated phospho-specific antibody is used at dilution 1:1000. Phosphorylation-site specificity of the antibody will be shown by binding of only the phosphorylated form of the target protein. Isolated phospho-specific polyclonal antibody does not (substantially) recognize the target protein when not phosphorylated at the appropriate phosphorylation site in the non-stimulated cells (e.g. TSN is not bound when not phosphorylated at tyrosine 210).

In order to confirm the specificity of the isolated antibody, different cell lysates containing various phosphorylated signal transduction proteins other than the target protein are prepared. The Western blot assay is performed again using these cell lysates. The phospho-specific polyclonal antibody isolated as described above is used (1:1000 dilution) to test reactivity with the different phosphorylated non-target proteins on Western blot membrane. The phospho-specific antibody does not significantly cross-react with other phosphorylated signal transduction proteins, although occasionally slight binding with a highly homologous phosphorylation-site on another protein may be observed. In such case the antibody may be further purified using affinity chromatography, or the specific immunoreactivity cloned by rabbit hybridoma technology.

### Example 3

### Production of Phospho-Specific Monoclonal Antibodies for the Detection of Target Signal Protein/Polypepetide Phosphorylation

Monoclonal antibodies that specifically bind a target signal protein/polypepetide only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, and harvesting spleen cells from such animals to produce fusion hybridomas, as further described below. Production of exemplary monoclonal antibodies is provided below.

### A. WRN (Tyrosine 849).

An 11 amino acid phospho-peptide antigen, DMESYy*QEIGR (where y*=phosphotyrosine) that corresponds to the sequence encompassing the tyrosine 849 phosphorylation site in human WRN chromatin or DNA binding/repair/replication protein (see Row 51 of Table 1 (SEQ ID NO: 50)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal WRN (tyr 849) antibodies as described in Immunization/Fusion/Screening below.

### B. SPTA1 (Tyrosine 1538).

An 11 amino acid phospho-peptide antigen, DATNIQRKy*LK (where y*=phosphotyrosine) that corresponds to the sequence encompassing the tyrosine 1538 phosphorylation site in human SPTA1 cytoskeletal protein (see Row 63 of Table 1 (SEQ ID NO: 62)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal SPTA1 (tyr1538) antibodies as described in Immunization/Fusion/Screening below.

C. SPTBN1 (tyrosine 1667).

A 15 amino acid phospho-peptide antigen, VDKLy*AGLKDLAEER (where y*=phosphotyrosine) that corresponds to the sequence encompassing the tyrosine 1667 phosphorylation site in human SPTBN1 cytoskeletal protein (see Row 61 of Table 1 (SEQ ID NO: 60), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal SPTBN1 (tyr1667) antibodies as described in Immunization/Fusion/Screening below.

### Immunization/Fusion/Screening.

A synthetic phospho-peptide antigen as described in A-C above is coupled to KLH, and BALB/C mice are injected intradermally (ID) on the back with antigen in complete Freunds adjuvant (e.g. 50 μg antigen per mouse). The mice are boosted with same antigen in incomplete Freund adjuvant (e.g. 25 μg antigen per mouse) every three weeks. After the fifth boost, the animals are sacrificed and spleens are harvested.

Harvested spleen cells are fused to SP2/0 mouse myeloma fusion partner cells according to the standard protocol of Kohler and Milstein (1975). Colonies originating from the fusion are screened by ELISA for reactivity to the phospho-peptide and non-phospho-peptide forms of the antigen and by Western blot analysis (as described in Example 1 above). Colonies found to be positive by ELISA to the phospho-peptide while negative to the non-phospho-peptide are further characterized by Western blot analysis. Colonies found to be positive by Western blot analysis are subcloned by limited dilution. Mouse ascites are produced from a single clone obtained from subcloning, and tested for phospho-specificity (against the WRN, SFTA1 or SPTBN1 phospho-peptide antigen, as the case may be) on ELISA. Clones identified as positive on Western blot analysis using cell culture supernatant as having phospho-specificity, as indicated by a strong band in the induced lane and a weak band in the uninduced lane of the blot, are isolated and subcloned as clones producing monoclonal antibodies with the desired specificity.

Ascites fluid from isolated clones may be further tested by Western blot analysis. The ascites fluid should produce similar results on Western blot analysis as observed previously with the cell culture supernatant, indicating phospho-specificity against the phosphorylated target (e.g. SPTA1 phosphorylated at tyrosine 1538).

### Example 4

### Production and Use of Aqua Peptides for the Quantification of Target Signal Protein/Polypepetide Phosphorylation

Heavy-isotope labeled peptides (AQUA peptides (internal standards)) for the detection and quantification of a target signal protein/polypepetide only when phosphorylated at the respective phosphorylation site disclosed herein (see Table 1/FIG. 2) are produced according to the standard AQUA methodology (see Gygi et al., Gerber et al., supra.) methods by first constructing a synthetic peptide standard corresponding to the phosphorylation site sequence and incorporating a heavy-isotope label. Subsequently, the MSⁿ and LC-SRM signature of the peptide standard is validated, and the AQUA peptide is used to quantify native peptide in a biological sample, such as a digested cell extract. Production and use of exemplary AQUA peptides is provided below.

### A. VASP (Tyrosine 15).

An AQUA peptide comprising the sequence, ATVMLy*DDGNKR (y*=phosphotyrosine; sequence incorporating ¹⁴C/¹⁵N-labeled leucine (indicated by bold L), which corresponds to the tyrosine 16 phosphorylation site in human VASP cytoskeletal protein (see Row 79 in Table 1 (SEQ ID NO: 78)), is constructed according to standard synthesis techniques using, e.g., a Rainin/Protein Technologies, Inc., Symphony peptide synthesizer (see Merrifield, supra.) as further described below in Synthesis & MS/MS Signature. The VASP (tyr 16) AQUA peptide is then spiked into a biological sample to quantify the amount of phosphorylated VASP (tyr 16) in the sample, as further described below in Analysis & Quantification.

### B. TOP2B (Tyrosine 230).

An AQUA peptide comprising the sequence IKHFDGEDy*TCITFTQPDLSK (y*=phosphotyrosine; sequence incorporating ¹⁴C/¹⁵N-labeled leucine (indicated by bold L), which corresponds to the tyrosine 230 phosphorylation site in human TOP2B chromatin or DNA binding/repair/replication protein (see Row 42 in Table 1 (SEQ ID NO: 41)), is constructed according to standard synthesis techniques using, e.g., a Rainin/Protein Technologies, Inc., Symphony peptide synthesizer (see Merrifield, supra.) as further described below in Synthesis & MS/MS Signature. The TOP2B (tyr230) AQUA peptide is then spiked into a biological sample to quantify the amount of phosphorylated TOP2B (tyr230) in the sample, as further described below in Analysis & Quantification.

### C. PKCD (Tyrosine 374)

An AQUA peptide comprising the sequence GRGEy*FAIK (y*=phosphotyrosine; sequence incorporating ¹⁴C/¹⁵N-labeled phenylalanine (indicated by bold F), which corresponds to the tyrosine 374 phosphorylation site in human PKCD protein kinase (Ser/Thr) (see Row 123 in Table 1 (SEQ ID NO: 122)), is constructed according to standard synthesis techniques using, e.g., a Rainin/Protein Technologies, Inc., Symphony peptide synthesizer (see Merrifield, supra.) as further described below in Synthesis & MS/MS Signature. The PKCD (tyr374) AQUA peptide is then spiked into a biological sample to quantify the amount of phosphorylated PKCD (tyr374) in the sample, as further described below in Analysis & Quantification.

### D. TAGLN3 (Tyrosine 192).

An AQUA peptide comprising the sequence, GASQAGMTGy*GMPR (y*=phosphotyrosine; sequence incorporating ¹⁴C/¹⁵N-labeled proline (indicated by bold P), which corresponds to the tyrosine 133 phosphorylation site in human TAGLN3 cytoskeletal protein (see Row 66 in Table 1 (SEQ ID NO: 65)), is constructed according to standard synthesis techniques using, e.g., a Rainin/Protein Technologies, Inc., Symphony peptide synthesizer (see Merrifield, supra.) as further described below in Synthesis & MS/MS Signature. The TAGLN3 (tyr192) AQUA peptide is then spiked into a biological sample to quantify the amount of phosphorylated TAGLN3 (tyr192) in the sample, as further described below in Analysis & Quantification.

### Synthesis & MS/MS Spectra.

Fluorenylmethoxycarbonyl (Fmoc)-derivatized amino acid monomers may be obtained from AnaSpec (San Jose, Calif.). Fmoc-derivatized stable-isotope monomers containing one ¹⁵N and five to nine ¹³C atoms may be obtained from Cambridge Isotope Laboratories (Andover, Mass.). Preloaded Wang resins may be obtained from Applied Biosystems. Synthesis scales may vary from 5 to 25 μmol. Amino acids are activated in situ with 1-H-benzotriazolium, 1-bis(dimethylamino) methylene]-hexafluorophosphate (1-),3-oxide:1-hydroxybenzotriazole hydrate and coupled at a 5-fold molar excess over peptide. Each coupling cycle is followed by capping with acetic anhydride to avoid accumulation of one-residue deletion peptide by-products. After synthesis peptide-resins are treated with a standard scavenger-containing trifluoroacetic acid (TFA)-water cleavage solution, and the peptides are precipitated by addition to cold ether. Peptides (i.e. a desired AQUA peptide described in A-D above) are purified by reversed-phase C18 HPLC using standard TFA/acetonitrile gradients and characterized by matrix-assisted laser desorption ionization-time of flight (Biflex III, Bruker Daltonics, Billerica, Mass.) and ion-trap (ThermoFinnigan, LCQ DecaXP) MS.

MS/MS spectra for each AQUA peptide should exhibit a strong γ-type ion peak as the most intense fragment ion that is suitable for use in an SRM monitoring/analysis. Reverse-phase microcapillary columns (0.1 Å˜150-220 mm) are prepared according to standard methods. An Agilent 1100 liquid chromatograph may be used to develop and deliver a solvent gradient 10.4% acetic acid/0.005% heptafluorobutyric acid (HFBA)/7% methanol and 0.4% acetic acid/0.005% HFBA/65% methanol/35% acetonitrile) to the microcapillary column by means of a flow splitter. Samples are then directly loaded onto the microcapillary column by using a FAMOS inert capillary autosampler (LC Packings, San Francisco) after the flow split. Peptides are reconstituted in 6% acetic acid/0.01% TFA before injection.

### Analysis & Quantification.

Target protein (e.g. a phosphorylated protein of A-D above) in a biological sample is quantified using a validated AQUA peptide (as described above). The IAP method is then applied to the complex mixture of peptides derived from proteolytic cleavage of crude cell extracts to which the AQUA peptides have been spiked in.

LC-SRM of the entire sample is then carried out. MS/MS may be performed by using a ThermoFinnigan (San Jose, Calif.) mass spectrometer (LTQ ion trap or TSQ Quantum triple quadrupole). On the LTQ, parent ions are isolated at 1.6 m/z width, the ion injection time being limited to 100 ms per microscan, with one microscans per peptide, and with an AGC setting of 1×10⁵; on the Quantum, Q1 is kept at 0.4 and Q3 at 0.8 m/z with a scan time of 200 ms per peptide. On both instruments, analyte and internal standard are analyzed in alternation within a previously known reverse-phase retention window; well-resolved pairs of internal standard and analyte are analyzed in separate retention segments to improve duty cycle. Data are processed by integrating the appropriate peaks in an extracted ion chromatogram (60.15 m/z from the fragment monitored) for the native and internal standard, followed by calculation of the ratio of peak areas multiplied by the absolute amount of internal standard (e.g., 384 fmol).

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. An isolated phosphorylation site-specific antibody that specifically binds a human signaling protein selected from Column A of Table 1, Rows 123, 66, 30, 140 and 194 only when phosphorylated at the tyrosine listed in corresponding Column D of Table 1, comprised within the phosphorylatable peptide sequence listed in corresponding Column E of Table 1 (SEQ ID NOs: 122, 65, 29, 139 and 193), wherein said antibody does not bind said signaling protein when not phosphorylated at said tyrosine.

47. An isolated phosphorylation site-specific antibody that specifically binds a human signaling protein selected from Column A of Table 1, Rows 123, 66, 30, 140 and 194 only when not phosphorylated at the tyrosine listed in corresponding Column D of Table 1, comprised within the phosphorylatable peptide sequence listed in corresponding Column E of Table 1 (SEQ ID NOs: 122, 65, 29, 139 and 193), wherein said antibody does not bind said signaling protein when phosphorylated at said tyrosine.

48. A method selected from the group consisting of:
(a) a method for detecting a human signaling protein selected from Column A of Table 1, Rows 123, 66, 30, 140 and 194 wherein said human signaling protein is phosphorylated at the tyrosine listed in corresponding Column D of Table 1, comprised within the phosphorylatable peptide sequence listed in corresponding Column E of Table 1 (SEQ ID NOs: 122, 65, 29, 139 and 193), comprising the step of adding an isolated phosphorylation-specific antibody according to claim 46, to a sample comprising said human signaling protein under conditions that permit the binding of said antibody to said human signaling protein, and detecting bound antibody;
(b) a method for quantifying the amount of a human signaling protein listed in Column A of Table 1, Rows 123, 66, 30, 140 and 194 that is phosphorylated at the corresponding tyrosine listed in Column D of Table 1, comprised within the phosphorylatable peptide sequence listed in corresponding Column E of Table 1 (SEQ ID NOs: 122, 65, 29, 139 and 193), in a sample using a heavy-isotope labeled peptide (AQUA™ peptide), said labeled peptide comprising a phosphorylated tyrosine at said corresponding lysine listed Column D of Table 1, comprised within the phosphorylatable peptide sequence listed in corresponding Column E of Table 1 as an internal standard; and
(c) a method comprising step (a) followed by step (b).

49. The method of claim 48, wherein said isolated phosphorylation-specific antibody is capable of specifically binding PKCD only when phosphorylated at Y374, comprised within the phosphorylatable peptide sequence listed in Column E, Row 123, of Table 1 (SEQ ID NO: 122), wherein said antibody does not bind said protein when not phosphorylated at said tyrosine.

50. The method of claim 48, wherein said isolated phosphorylation-specific antibody is capable of specifically binding PKCD only when not phosphorylated at Y374, comprised within the phosphorylatable peptide sequence listed in Column E, Row 123, of Table 1 (SEQ ID NO: 122), wherein said antibody does not bind said protein when phosphorylated at said tyrosine.

51. The method of claim 48, wherein said isolated phosphorylation-specific antibody is capable of specifically binding TAGLN3 only when phosphorylated at Y192, comprised within the phosphorylatable peptide sequence listed in Column E, Row 66, of Table 1 (SEQ ID NO: 65), wherein said antibody does not bind said protein when not phosphorylated at said tyrosine.

52. The method of claim 48, wherein said isolated phosphorylation-specific antibody is capable of specifically binding TAGLN3 only when not phosphorylated at Y192, comprised within the phosphorylatable peptide sequence listed in Column E, Row 66, of Table 1 (SEQ ID NO: 65), wherein said antibody does not bind said protein when phosphorylated at said tyrosine.

53. The method of claim 48, wherein said isolated phosphorylation-specific antibody is capable of specifically binding HSP90B only when phosphorylated at Y192, comprised within the phosphorylatable peptide sequence listed in Column E, Row 30, of Table 1 (SEQ ID NO: 29), wherein said antibody does not bind said protein when not phosphorylated at said tyrosine.

54. The method of claim 48, wherein said isolated phosphorylation-specific antibody is capable of specifically binding HSP90B only when not phosphorylated at Y192, comprised within the phosphorylatable peptide sequence listed in Column E, Row 30, of Table 1 (SEQ ID NO: 29), wherein said antibody does not bind said protein when phosphorylated at said tyrosine.

55. The method of claim 48, wherein said isolated phosphorylation-specific antibody is capable of specifically binding Tyro3 only when phosphorylated at Y685, comprised within the phosphorylatable peptide sequence listed in Column E, Row 140, of Table 1 (SEQ ID NO: 139), wherein said antibody does not bind said protein when not phosphorylated at said tyrosine.

56. The method of claim 48, wherein said isolated phosphorylation-specific antibody is capable of specifically binding Tyro3 only when not phosphorylated at Y685, comprised within the phosphorylatable peptide sequence listed in Column E, Row 140, of Table 1 (SEQ ID NO: 139), wherein said antibody does not bind said protein when phosphorylated at said tyrosine.

57. The method of claim 48, wherein said isolated phosphorylation-specific antibody is capable of specifically binding STAT5B only when phosphorylated at Y683, comprised within the phosphorylatable peptide sequence listed in Column E, Row 194, of Table 1 (SEQ ID NO: 193), wherein said antibody does not bind said protein when not phosphorylated at said tyrosine.

58. The method of claim 48, wherein said isolated phosphorylation-specific antibody is capable of specifically binding STAT5B only when not phosphorylated at Y683, comprised within the phosphorylatable peptide sequence listed in Column E, Row 194, of Table 1 (SEQ ID NO: 193), wherein said antibody does not bind said protein when phosphorylated at said tyrosine.
